(19) United States
(12) Patent Application Publication
(10) Pub. No.: US 2020/0121738 A1
(43) Pub. Date: Apr. 23, 2020

(54) METHODS AND COMPOSITIONS RELATING TO ISOLATED AND PURIFIED MICROBES

(71) Applicant: Pendulum Therapeutics, Inc., San Francisco, CA (US)

(72) Inventors: Colleen CUTCLIFFE, Menlo Park, CA (US); John S. EID, San Francisco, CA (US); James H. Bullard, San Francisco, CA (US); Marcus F. SCHICKLBERGER, Richmond, CA (US); Andrew T. CHENG, San Mateo, CA (US); Orville G. KOLTERMAN, La Jolla, CA (US); Tomer ALTMAN, San Francisco, CA (US)

(21) Appl. No.: 16/431,257

(22) Filed: Jun. 4, 2019

Related U.S. Application Data
(63) Continuation of application No. PCT/US2017/064973, filed on Dec. 6, 2017.
(60) Provisional application No. 62/502,483, filed on May 5, 2017.
(60) Provisional application No. 62/430,891, filed on Dec. 6, 2016.

(51) Int. Cl. A61K 35/741; C12N 1/20; A61K 9/00; A61K 9/19; A61K 31/733; A23L 33/135

(52) U.S. Cl. CPC A61K 35/741; C12N 1/20; A61K 9/0053; A23Y 2300/45; A61K 31/733; A23L 33/135; A23V 2002/00; A61K 9/19

(57) ABSTRACT

The present disclosure provides a method for formulating a composition of isolated and purified microbes. The present disclosure provides methods for formulating a composition for administration to a subject in need thereof. The method can comprise obtaining a mixture that is substantially dry and comprises about 10% or less residual moisture. The mixture can comprise a population of isolated and purified microbes and a pharmaceutically acceptable carrier. The population can comprise one or more obligate anaerobes that are oxygen-stable. The methods can further comprise encapsulating said mixture in an enteric-coated capsule for delivery to said subject.

### CROSS-REFERENCE

This application claims priority to U.S. Provisional Application No. 62/430,891, filed Dec. 6, 2016, and U.S. Provisional Application No. 62/502,483, filed May 5, 2017, each of which is entirely incorporated herein by reference.

### BACKGROUND

The body of an individual can be inhabited by trillions of microbes across various locations. These populations across various locations are often referred to as microbiomes. Microbiomes can play a role in many health conditions and diseases. Despite the interrelation between microbiomes and health, the complexity of the various microbiomes, as well as difficulties in characterizing, categorizing, and analyzing microbiome constituents can make understanding microbiomes challenging. These challenges can present hurdles in the development of diagnostic and therapeutic applications for microbiome-related health conditions and diseases.

### BIOLOGICAL DEPOSITS

This application contains a reference to a deposit of biological material. The following biological materials have been deposited with the American Type Culture Collection (ATCC), in Manassas, Va., and bear the following designations, accession numbers and dates of deposit: Clostridium beijerinckii; (PTA-123634, deposited Dec. 14, 2016); Clostridium butyricum; (PTA-123635, deposited Dec. 14, 2016).

### SUMMARY

Disclosed herein is a composition for administration to a subject in need thereof, comprising: a population of one or more isolated and purified microbes comprising one or more obligate anaerobes, wherein the composition remains stable when stored at a temperature of 4 degrees Celsius or room temperature for 2 weeks or more as determined by measuring a first cell count of viable microbes or active cells at a first time using flow cytometry and a second cell count of viable microbes or active cells at a second time using flow cytometry, wherein the first time and the second time are 2 weeks or more apart, and further wherein the second cell count is at least 0.1% of the first cell count.

In another aspect, is a composition for administration to a subject in need thereof, comprising: a population of one or more isolated and purified microbes, the population comprising one or more obligate anaerobes that are oxygen-stable such that at least 0.1% of the one or more obligate anaerobes are viable microbes or active cells when stored at 4 degrees Celsius or room temperature for a period of 14 days, wherein the population is substantially dry and comprises about 5% or less residual moisture.

In one embodiment, at least 1% of the one or more obligate anaerobes are viable or active cells. In one embodiment, the composition is encapsulated and comprises at least 20% of the one or more obligate anaerobes are viable or active cells In one embodiment, 5% to 75% of the one or more obligate anaerobes are viable microbes or active cells. In one embodiment, 10% to 50% of the one or more obligate anaerobes are viable microbes or active cells. In one embodiment, the composition comprises an amount of at least 10⁸ active cells/g of one or more microbes in the population. Room temperature is 20 to 25 degrees Celsius. Room temperature can be 20 degrees Celsius.

In yet another aspect, a composition is provided for administration to a subject in need thereof, comprising a population of one or more isolated and purified microbes, wherein the composition comprises the following properties: a) at least 1.0×10⁸ active cells/g, and b) the composition comprises no more than 5.0 mcg/g of arsenic, no more than 3.3 mcg/g of lead, no more than 5.0 mcg/g of mercury, and no more than 1.6 mcg/g of cadmium.

In yet another aspect, a composition is provided for administration to a subject in need thereof, comprising a population of one or more isolated and purified microbes, wherein the composition comprises the following properties: a) about 8.2×10⁹ active cells/g, and b) the composition comprises no more than about 0.02 mcg/g of arsenic, no more than about 0.2 mcg/g of lead, no more than about 0.01 mcg/g of mercury, and no more than about 0.12 mcg/g of cadmium.

In one embodiment, the composition is beige to dark tan in color. In one embodiment, the the composition is tan in color.

In one embodiment, the composition is a powder.

In one embodiment, one or more oxygen-stable obligate anaerobes are viable in greater than 5 μM oxygen. In one embodiment, one or more oxygen-stable obligate anaerobes are viable in conditions comprising 20% or greater oxygen, by volume. In one embodiment, at least one of the one or more obligate oxygen-stable anaerobes are capable of growing in conditions having 5 μM or less of dissolved oxygen.

In one embodiment, the composition comprises 90% or greater non-sporulated obligate anaerobes.

In one embodiment, the population can comprise one or more isolated and purified microbes selected from the group consisting of: Akkermansia muciniphila, Anaerostipes caccae, Bifidobacterium adolescentis, Bifidobacterium bifidum, Bifidobacterium infantis, Bifidobacterium longum, Butyrivibrio fibrisolvens, Clostridium acetobutylicum, Clostridium aminophilum, Clostridium beijerinckii, Clostridium butyricum, Clostridium colinum, Clostridium coccoides, Clostridium indolis, Clostridium nexile, Clostridium orbiscindens, Clostridium propionicum, Clostridium xylanolyticum, Enterococcus faecium, Eubacterium hallii, Eubacterium rectale, Faecalibacterium prausnitzii, Fibrobacter succinogenes, Lactobacillus acidophilus, Lactobacillus brevis, Lactobacillus bulgaricus, Lactobacillus casei, Lactobacillus caucasicus, Lactobacillus fermentum, Lactobacillus helveticus, Lactobacillus lactis, Lactobacillus plantarum, Lactobacillus reuteri, Lactobacillus rhamnosus, Oscillospira guilliermondii, Roseburia cecicola, Roseburia inulinivorans, Ruminococcus flavefaciens, Ruminococcus gnavus, Ruminococcus obeum, Stenotrophomonas nitritireducens, Streptococcus cremoris, Streptococcus faecium, Streptococcus infantis, Streptococcus mutans, Streptococcus thermophilus, Anaerofustis stercorihominis, Anaerostipes hadrus, Anaerotruncus colihominis, Clostridium sporogenes, Clostridium tetani, Coprococcus, Coprococcus eutactus, Eubacterium cylindroides, Eubacterium dolichum, Eubacterium ventriosum, Roseburia faeccis, Roseburia hominis, Roseburia intestinalis, Lacatobacillus bifidus, Lactobacillus johnsonii, Lactobacilli, Acidaminococcus fermentans, Acidaminococcus intestine, Blautia hydrogenotrophica, Citrobacter amalonaticus, Citrobacter freundii, Clostridium aminobutyricum Clostridium bartlettii, Clostridium cochlearium, Clostridium kluyveri, Clostridium limosum, Clostridium malenominatum, Clostridium pasteurianum, Clostridium peptidivorans, Clostridium saccharobutylicum, Clostridium sporosphaeroides, Clostridium sticklandii, Clostridium sub terminale, Clostridium symbiosum, Clostridium tetanomorphum, Eubacterium oxidoreducens, Eubacterium pyruvativorans, Methanobrevibacter smithii, Morganella morganii, Peptomphilus asaccharolyticus, and Peptostreptococcus, and any combination thereof.

In one embodiment, the population can comprise 2 or more, 3 or more, 2 to 10, 3 to 7, up to 7, or up to 10 isolated and purified microbes selected from the group consisting of: Akkermansia muciniphila, Anaerostipes caccae, Bifidobacterium adolescentis, Bifidobacterium bifidum, Bifidobacterium infantis, Bifidobacterium longum, Butyrivibrio fibrisolvens, Clostridium acetobutylicum, Clostridium aminophilum, Clostridium beijerinckii, Clostridium butyricum, Clostridium colinum, Clostridium coccoides, Clostridium indolis, Clostridium nexile, Clostridium orbiscindens, Clostridium propionicum, Clostridium xylanolyticum, Enterococcus faecium, Eubacterium hallii, Eubacterium rectale, Faecalibacterium prausnitzii, Fibrobacter succinogenes, Lactobacillus acidophilus, Lactobacillus brevis, Lactobacillus bulgaricus, Lactobacillus casei, Lactobacillus caucasicus, Lactobacillus fermentum, Lactobacillus helveticus, Lactobacillus lactis, Lactobacillus plantarum, Lactobacillus reuteri, Lactobacillus rhamnosus, Oscillospira guilliermondii, Roseburia cecicola, Roseburia inulinivorans, Ruminococcus flavefaciens, Ruminococcus gnavus, Ruminococcus obeum, Stenotrophomonas nitritireducens, Streptococcus cremoris, Streptococcus faecium, Streptococcus infantis, Streptococcus mutans, Streptococcus thermophilus, Anaerofustis stercorihominis, Anaerostipes hadrus, Anaerotruncus colihominis, Clostridium sporogenes, Clostridium tetani, Coprococcus, Coprococcus eutactus, Eubacterium cylindroides, Eubacterium dolichum, Eubacterium ventriosum, Roseburia faeccis, Roseburia hominis, Roseburia intestinalis, Lacatobacillus bifidus, Lactobacillus johnsonii, Lactobacilli, Acidaminococcus fermentans, Acidaminococcus intestine, Blautia hydrogenotrophica, Citrobacter amalonaticus, Citrobacter freundii, Clostridium aminobutyricum Clostridium bartlettii, Clostridium cochlearium, Clostridium kluyveri, Clostridium limosum, Clostridium malenominatum, Clostridium pasteurianum, Clostridium peptidivorans, Clostridium saccharobutylicum, Clostridium sporosphaeroides, Clostridium sticklandii, Clostridium subterminale, Clostridium symbiosum, Clostridium tetanomorphum, Eubacterium oxidoreducens, Eubacterium pyruvativorans, Methanobrevibacter smithii, Morganella morganii, Peptomphilus asaccharolyticus, and Peptostreptococcus, and any combination thereof.

In one embodiment, the composition further comprises a prebiotic. In one embodiment, the prebiotic comprises inulin. In one embodiment, the inulin is present in an amount of at least about 50 mg/ml.

In one embodiment, the composition further comprises inulin, sucrose, trehalose, glycerin, maltodextrin, and hydroxypropyl methylcellulose.

In one embodiment, the composition is formulated for oral delivery. In one embodiment, the composition is a tablet.

In one embodiment, the composition is a suppository.

In yet another aspect, the present disclosure provides a method for obtaining a composition of the as described herein, the method comprising: (a) cultivating the population of one or more isolated and purified microbes comprising one or more obligate anaerobes; and (b) lyophilizing the population, thereby generating the one or more obligate anaerobes that are oxygen-stable.

In one embodiment, the method further comprises encapsulating the population.

In one embodiment, one or more obligate anaerobes that are oxygen-stable are lyophilized with a cryoprotectant selected from the group consisting of: glycerol, trehalose, sucrose, inulin, water, vegetable media, skim milk, dextran, glutamic acid, histidine, mannitol, and any combination thereof.

In one embodiment, the cryoprotectant comprises 10% glycerol.

In one embodiment, lyophilizing one or more obligate anaerobes that are oxygen-stable yields a dry powder.

In one embodiment, cultivating comprises growing in media comprising N-Acetylglucosamine. In one embodiment, the media comprises an effective amount of dextrose.

In one embodiment, the media further comprises an effective amount of a salt. In one embodiment, the salt is selected from the group consisting of: ammonium chloride, calcium chloride, calcium chloride dihydrate, calcium chloride hexahydrate, calcium chloride decahydrate, ferric nitrate, magnesium sulfate monohydrate, magnesium sulfate pentahydrate, magnesium sulfate heptahydrate, magnesium chloride, magnesium sulfate, magnesium sulfate nonahydrate, meridianiite, magnesium sulfate dodecahydrate, potassium chloride, potassium hydrogen phosphate, potassium dihydrogen phosphate, monopotassium phosphate, Dipotassium phosphate, potassium sulfate, sodium hydrogen carbonate, sodium hydrogen phosphate, sodium chloride, and any combination thereof. In one embodiment, the salt is selected from the group consisting of: dipotassium phosphate, calcium chloride, magnesium sulfate, monopotassium phosphate, sodium bicarbonate, sodium chloride, and any combination thereof.

In one embodiment, the media further comprises a vitamin. In one embodiment, the vitamin is selected from the group consisting of: D-biotin, calcium pantothenate, myinositol, p-aminobenzoic acid, folic acid, pyridoxine hydrochloride, pyridoxine (B6), biotin, riboflavin, lipoic acid, thiamine dichloride, mercaptoethane sulfonic acid, nicotinic acid, pantothenic acid, vitamin A, vitamin B12, vitamin K, riboflavin (B2), thiamine (B1), K-Ca-pantothenate, choline chloride, i-inositol, niacinamide, pyridoxal HCl, pyridoxine HCl, thiamine HCl, para-aminobenzoic acid, niacin, ascorbic acid, a-Tocopherol phosphate, calciferol, menadione, nicotinic acid, and any combination thereof. In one embodiment, the vitamin is selected from the group consisting of: D-biotin, calcium pantothenate, myoinositol, P-aminobenzoic acid, pyridoxine hydrochloride, riboflavin, thiamine dichloride, Vitamin B12, nicotinic acid, and any combination thereof.

In one embodiment, the media further comprises a surfactant.

In one embodiment, the media further comprises an amino acid source.

In one embodiment, the amino acid source is L-arginine, L-cysteine, L-cysteine, L-histidine, L-isoleucine, L-leucine, L-lysine, L-methionine, L-phenylalanine, L-threomine, L-typtophan, L-tyrosine, L-valine, L-alanine, L-asparagine, L-aspartic acid, L-glutamic acid, L-glutamine, glycine, L-proline, L-serine, and L-hydroxyproline, peptone, soya peptone, HiVeg Peptone #1, HiVeg Peptone #2, HiVeg Peptone #3, HiVeg Peptone #4, HiVeg Peptone #5, HiVeg Special Peptone, protease peptone, or a combination thereof.

In one embodiment, the media has a pH of about 7.0.

In still another aspect, the present disclosure provides for method of producing the composition disclosed herein and, additionally, further comprising administering a therapeutically effective amount to a subject in need thereof. In one aspect, the method comprises (a) obtaining the composition; and (b) administering a therapeutically effective amount of the composition to a subject in need thereof. The composition can be orally administered. The composition can be rectally administered.

In one embodiment, administering the therapeutically effective amount of the composition comprises administering one or more dosage forms daily for a period of at least 7 days.

In one embodiment, administering the therapeutically effective amount of the composition comprises administering one or more dosage forms daily for a period of 7 days to 14 days.

Disclosed herein is a method for formulating a composition for administration to a subject in need thereof. The method can comprise obtaining a mixture comprising a population of isolated and purified microbes, wherein the mixture can be substantially dry and can comprise about 5% or less residual moisture, wherein the population comprises one or more obligate anaerobes that are oxygen-stable, and encapsulating the mixture for delivery to the subject. In one embodiment, 0.1% or greater of the one or more oxygen-stable obligate anaerobes are viable. In one embodiment, the mixture can comprise about 90% or greater non-sporulated obligate anaerobes. In one embodiment, the mixture can be encapsulated in an enteric coated capsule. In one embodiment, the method can further comprise coating the mixture in an enteric coating. In one embodiment, the population can comprise an isolated and purified microbe with a ribosomal RNA (rRNA) sequence comprising at least about 85% sequence identity to a rRNA sequence from Akkermansia muciniphila. In one embodiment, the population can comprise an isolated and purified microbe with a ribosomal RNA (rRNA) sequence comprising at least about 85% sequence identity to a rRNA sequence from Bifidobacterium adolescentis. In one embodiment, the population can comprise an isolated and purified microbe with a ribosomal RNA (rRNA) sequence comprising at least about 85% sequence identity to a rRNA sequence from Bifidobacterium infantis. In one embodiment, the population can comprise an isolated and purified microbe with a ribosomal RNA (rRNA) sequence comprising at least about 85% sequence identity to a rRNA sequence from Bifidobacterium longum. In one embodiment, the population can comprise an isolated and purified microbe with a ribosomal RNA (rRNA) sequence comprising at least about 85% sequence identity to a rRNA sequence from Clostridium beijerinckii. In one embodiment, the population can comprise an isolated and purified microbe with a ribosomal RNA (rRNA) sequence comprising at least about 85% sequence identity to a rRNA sequence from Clostridium butyricum. In one embodiment, the population can comprise an isolated and purified microbe with a ribosomal RNA (rRNA) sequence comprising at least about 85% sequence identity to a rRNA sequence from Clostridium indolis. In one embodiment, the population can comprise an isolated and purified microbe with a ribosomal RNA (rRNA) sequence comprising at least about 85% sequence identity to a rRNA sequence from Eubacterium hallii. In one embodiment, the population can comprise an isolated and purified microbe with a ribosomal RNA (rRNA) sequence comprising at least about 85% sequence identity to a rRNA sequence from Faecalibacterium prausnitzii. In one embodiment, the population can comprise at least one microbe from genus Akkermansia and at least one microbe from a genus selected from the group consisting of: Eubacterium, Clostridium, Bifidobacterium, and Faecalibacterium. In one embodiment, the population of microbes can comprise at least two isolated and purified microbes from each of phylum Verrucomicrobia and phylum Actinobacteria. In one embodiment, the one or more oxygen-stable obligate anaerobes can be viable in about 5 μM or greater oxygen. In one embodiment, the one or more oxygen-stable obligate anaerobes can be viable in conditions comprising about 20% or greater oxygen, by volume. In one embodiment, at least one of the one or more obligate oxygen-stable anaerobes can grow in conditions having 5 μM or less of dissolved oxygen. In one embodiment, the population can comprise one or more isolated and purified microbes selected from the group consisting of: Akkermansia muciniphila, Anaerostipes caccae, Bifidobacterium adolescentis, Bifidobacterium bifidum, Bifidobacterium infantis, Bifidobacterium longum, Butyrivibrio fibrisolvens, Clostridium acetobutylicum, Clostridium aminophilum, Clostridium beijerinckii, Clostridium butyricum, Clostridium colinum, Clostridium coccoides, Clostridium indolis, Clostridium nexile, Clostridium orbiscindens, Clostridium propionicum, Clostridium xylanolyticum, Enterococcus faecium, Eubacterium hallii, Eubacterium rectale, Faecalibacterium prausnitzii, Fibrobacter succinogenes, Lactobacillus acidophilus, Lactobacillus brevis, Lactobacillus bulgaricus, Lactobacillus casei, Lactobacillus caucasicus, Lactobacillus fermentum, Lactobacillus helveticus, Lactobacillus lactis, Lactobacillus plantarum, Lactobacillus reuteri, Lactobacillus rhamnosus, Oscillospira guilliermondii, Roseburia cecicola, Roseburia inulinivorans, Ruminococcus flavefaciens, Ruminococcus gnavus, Ruminococcus obeum, Stenotrophomonas nitritireducens, Streptococcus cremoris, Streptococcus faecium, Streptococcus infantis, Streptococcus mutans, Streptococcus thermophilus, Anaerofustis stercorihominis, Anaerostipes hadrus, Anaerotruncus colihominis, Clostridium sporogenes, Clostridium tetani, Coprococcus, Coprococcus eutactus, Eubacterium cylindroides, Eubacterium dolichum, Eubacterium ventriosum, Roseburia faeccis, Roseburia hominis, Roseburia intestinalis, Lacatobacillus bifidus, Lactobacillus johnsonii, Lactobacilli, Acidaminococcus fermentans, Acidaminococcus intestine, Blautia hydrogenotrophica, Citrobacter amalonaticus, Citrobacter freundii, Clostridium aminobutyricum Clostridium bartlettii, Clostridium cochlearium, Clostridium kluyveri, Clostridium limosum, Clostridium malenominatum, Clostridium pasteurianum, Clostridium peptidivorans, Clostridium saccharobutylicum, Clostridium sporosphaeroides, Clostridium sticklandii, Clostridium subterminale, Clostridium symbiosum, Clostridium tetanomorphum, Eubacterium oxidoreducens, Eubacterium pyruvativorans, Methanobrevibacter smithii, Morganella morganii, Peptomphilus asaccharolyticus, and Peptostreptococcus, and any combination thereof. In one embodiment, the mixture can further comprise a prebiotic. In one embodiment, the prebiotic can comprise inulin. In one embodiment, the inulin can be present in an amount of at least about 50 mg/ml in the composition. In one embodiment, the mixture can be encapsulated for delivery to a small intestine, a large intestine, an ileum, or a combination thereof, of the subject. In one embodiment, the encapsulated mixture may not substantially release the population of isolated and purified microbes prior to a small intestine or a large intestine of the subject. In one embodiment, the capsule can dissolve at a pH greater than at least about pH 6.5. In one embodiment, the capsule can comprise one or more enteric coatings. In one embodiment, the composition can be formulated for oral delivery. In one embodiment, the encapsulated mixture can be stable at 4° C. for 2 weeks. In one embodiment, the encapsulated mixture can comprise an amount of at least about 10⁵ colony forming units (CFU) of one or more microbes in the population of isolated and purified microbes. In one embodiment, the encapsulated mixture can comprise an amount of at least about 0.1% viable microbes. In one embodiment, the subject can be human.

In one embodiment, the method can further comprise generating one or more isolated and purified oxygen-stable anaerobes. In one embodiment, the generating step can comprise lyophilizing one or more isolated and purified anaerobes. In one embodiment, the generated one or more isolated and purified anaerobes can be lyophilized with a cryoprotectant selected from the group consisting of: glycerol, trehalose, sucrose, inulin, water, vegetable media, skim milk, dextran, glutamic acid, histidine, mannitol, and any combination thereof. In one embodiment, the lyophilizing one or more isolated and purified anaerobes can yield a dry powder obtained without further processing. In one embodiment, the mixture can further comprise a pharmaceutically acceptable carrier. In one embodiment, the mixture can be a powder. In one embodiment, the powder can comprise particles with a uniform particle size. In one embodiment, particles in the powder can be non-cohesive.

In one embodiment, disclosed herein are compositions for administration to a subject in need thereof, comprising a) a population of one or more isolated and purified obligate anaerobes and b) a capsule enclosing the population therein, wherein the composition remains stable when stored at a temperature of 4 degrees Celsius for 2 weeks or more as determined by measuring, using flow cytometry, a first cell count of viable microbes at a first time and a second cell count of viable microbes at a second time, wherein the first time and the second time are up to 2 weeks apart and the composition can be stored at 4 degrees Celsius from the first time to the second time, and further wherein the second cell count is at least 60% of the first cell count. In one embodiment, the one or more isolated and purified obligate anaerobes can be oxygen stable. In one embodiment, the composition can further comprise a pharmaceutically acceptable carrier. In one embodiment, 0.1% or greater of the one or more isolated and purified obligate anaerobes can be viable. In one embodiment, the one or more isolated and purified obligate anaerobes can comprise about 90% or greater non-sporulated obligate anaerobes. In one embodiment, the population can comprise an isolated and purified microbe with a ribosomal RNA (rRNA) sequence comprising at least about 85% sequence identity to a rRNA sequence from Akkermansia muciniphila. In one embodiment, the population can comprise an isolated and purified microbe with a ribosomal RNA (rRNA) sequence comprising at least about 85% sequence identity to a rRNA sequence from Bifidobacterium adolescentis. In one embodiment, the population can comprise an isolated and purified microbe with a ribosomal RNA (rRNA) sequence comprising at least about 85% sequence identity to a rRNA sequence from Bifidobacterium infantis. In one embodiment, the population can comprise an isolated and purified microbe with a ribosomal RNA (rRNA) sequence comprising at least about 85% sequence identity to a rRNA sequence from Bifidobacterium longum. In one embodiment, the population can comprise an isolated and purified microbe with a ribosomal RNA (rRNA) sequence comprising at least about 85% sequence identity to a rRNA sequence from Clostridium beijerinckii. In one embodiment, the population can comprise an isolated and purified microbe with a ribosomal RNA (rRNA) sequence comprising at least about 85% sequence identity to a rRNA sequence from Clostridium butyricum. In one embodiment, the population can comprise an isolated and purified microbe with a ribosomal RNA (rRNA) sequence comprising at least about 85% sequence identity to a rRNA sequence from Clostridium indolis. In one embodiment, the population can comprise an isolated and purified microbe with a ribosomal RNA (rRNA) sequence comprising at least about 85% sequence identity to a rRNA sequence from Eubacterium hallii. In one embodiment, the population can comprise at least one microbe from genus Akkermansia and at least one microbe from a genus selected from the group consisting of: Eubacterium, Clostridium, Bifidobacterium, and Faecalibacterium. In one embodiment, the population of microbes can comprise at least two isolated and purified microbes from each of phylum Verrucomicrobia and phylum Actinobacteria. In one embodiment, the population can comprise one or more isolated and purified microbes selected from the group consisting of: Akkermansia muciniphila, Anaerostipes caccae, Bifidobacterium adolescentis, Bifidobacterium bifidum, Bifidobacterium infantis, Bifidobacterium longum, Butyrivibrio fibrisolvens, Clostridium acetobutylicum, Clostridium aminophilum, Clostridium beijerinckii, Clostridium butyricum, Clostridium colinum, Clostridium coccoides, Clostridium indolis, Clostridium nexile, Clostridium orbiscindens, Clostridium propionicum, Clostridium xylanolyticum, Enterococcus faecium, Eubacterium hallii, Eubacterium rectale, Faecalibacterium prausnitzii, Fibrobacter succinogenes, Lactobacillus acidophilus, Lactobacillus brevis, Lactobacillus bulgaricus, Lactobacillus casei, Lactobacillus caucasicus, Lactobacillus fermentum, Lactobacillus helveticus, Lactobacillus lactis, Lactobacillus plantarum, Lactobacillus reuteri, Lactobacillus rhamnosus, Oscillospira guilliermondii, Roseburia cecicola, Roseburia inulinivorans, Ruminococcus flavefaciens, Ruminococcus gnavus, Ruminococcus obeum, Stenotrophomonas nitritireducens, Streptococcus cremoris, Streptococcus faecium, Streptococcus infantis, Streptococcus mutans, Streptococcus thermophilus, Anaerofustis stercorihominis, Anaerostipes hadrus, Anaerotruncus colihominis, Clostridium sporogenes, Clostridium tetani, Coprococcus, Coprococcus eutactus, Eubacterium cylindroides, Eubacterium dolichum, Eubacterium ventriosum, Roseburia faeccis, Roseburia hominis, Roseburia intestinalis, Lacatobacillus bifidus, Lactobacillus johnsonii, Lactobacilli, Acidaminococcus fermentans, Acidaminococcus intestine, Blautia hydrogenotrophica, Citrobacter amalonaticus, Citrobacter freundii, Clostridium aminobutyricum Clostridium bartlettii, Clostridium cochlearium, Clostridium kluyveri, Clostridium limosum, Clostridium malenominatum, Clostridium pasteurianum, Clostridium peptidivorans, Clostridium saccharobutylicum, Clostridium sporosphaeroides, Clostridium sticklandii, Clostridium subterminale, Clostridium symbiosum, Clostridium tetanomorphum, Eubacterium oxidoreducens, Eubacterium pyruvativorans, Methanobrevibacter smithii, Morganella morganii, Peptoniphilus asaccharolyticus, and Peptostreptococcus, and any combination thereof. In one embodiment, the composition can further comprise a prebiotic. In one embodiment, the prebiotic can comprise inulin. In one embodiment, the inulin can be present in an amount of at least about 50 mg/ml in the composition. In one embodiment, the capsule can comprise an enteric coating. In one embodiment, the composition can further comprise an enteric coating. In one embodiment, the capsule can be configured for delivery to a small intestine, a large intestine, an ileum, or a combination thereof, of the subject. In one embodiment, the capsule may not substantially release the population of isolated and purified microbes prior to a small intestine or a large intestine of the subject. In one embodiment, the capsule can dissolve at a pH greater than at least about pH 6.5. In one embodiment, the pharmaceutical composition can comprise one or more enteric coatings. In one embodiment, the composition can be stable at 4° C. for 2 weeks. In one embodiment, the composition can comprise an amount of at least about 10⁵ colony forming units (CFU) of one or more microbes in the population. In one embodiment, the encapsulated mixture can comprise an amount of at least about 0.1% viable microbes. In one embodiment, the subject can be human. In one embodiment, the mixture can be a powder. In one embodiment, the powder can comprise particles with a uniform particle size. In one embodiment, particles in the powder can be non-cohesive.

In one embodiment, disclosed herein are compositions for administration to a subject in need thereof, the composition comprising: a) a population of one or more isolated and purified obligate anaerobes and b) a capsule enclosing the population therein, wherein the composition is stable, as determined by using gas chromatography with a flame ionization detector to measure short-chain fatty acid production of the population of one or more isolated an purified obligate anaerobes of the capsule at a first time and at a second time, wherein the first time and the second time can be 2 weeks or more apart and the composition can be stored at 4 degrees Celsius from the first time to the second time, and further wherein short chain fatty acid production measured at the first time can be about 60% or more than short chain fatty acid production measured at the second time. In one embodiment, the one or more isolated and purified obligate anaerobes can be oxygen stable. In one embodiment, the composition can further comprise a pharmaceutically acceptable carrier. In one embodiment, 0.1% or greater of the one or more isolated and purified obligate anaerobes can be viable. In one embodiment, the one or more isolated and purified obligate anaerobes can comprise about 90% or greater non-sporulated obligate anaerobes. In one embodiment, the population can comprise an isolated and purified microbe with a ribosomal RNA (rRNA) sequence comprising at least about 85% sequence identity to a rRNA sequence from Akkermansia muciniphila. In one embodiment, the population can comprise an isolated and purified microbe with a ribosomal RNA (rRNA) sequence comprising at least about 85% sequence identity to a rRNA sequence from Bifidobacterium adolescentis. In one embodiment, the population can comprise an isolated and purified microbe with a ribosomal RNA (rRNA) sequence comprising at least about 85% sequence identity to a rRNA sequence from Bifidobacterium infantis. In one embodiment, the population can comprise an isolated and purified microbe with a ribosomal RNA (rRNA) sequence comprising at least about 85% sequence identity to a rRNA sequence from Bifidobacterium longum. In one embodiment, the population can comprise an isolated and purified microbe with a ribosomal RNA (rRNA) sequence comprising at least about 85% sequence identity to a rRNA sequence from Clostridium beijerinckii. In one embodiment, the population can comprise an isolated and purified microbe with a ribosomal RNA (rRNA) sequence comprising at least about 85% sequence identity to a rRNA sequence from Clostridium butyricum. In one embodiment, the population can comprise an isolated and purified microbe with a ribosomal RNA (rRNA) sequence comprising at least about 85% sequence identity to a rRNA sequence from Clostridium indolis. In one embodiment, the population can comprise an isolated and purified microbe with a ribosomal RNA (rRNA) sequence comprising at least about 85% sequence identity to a rRNA sequence from Eubacterium hallii. In one embodiment, the population can comprise at least one microbe from genus Akkermansia and at least one microbe from a genus selected from the group consisting of: Eubacterium, Clostridium, Bifidobacterium, and Faecalibacterium. In one embodiment, the population of microbes can comprise at least two isolated and purified microbes from each of phylum Verrucomicrobia and phylum Actinobacteria. In one embodiment, the population can comprise one or more isolated and purified microbes selected from the group consisting of: Akkermansia muciniphila, Anaerostipes caccae, Bifidobacterium adolescentis, Bifidobacterium bifidum, Bifidobacterium infantis, Bifidobacterium longum, Butyrivibrio fibrisolvens, Clostridium acetobutylicum, Clostridium aminophilum, Clostridium beijerinckii, Clostridium butyricum, Clostridium colinum, Clostridium coccoides, Clostridium indolis, Clostridium nexile, Clostridium orbiscindens, Clostridium propionicum, Clostridium xylanolyticum, Enterococcus faecium, Eubacterium hallii, Eubacterium rectale, Faecalibacterium prausnitzii, Fibrobacter succinogenes, Lactobacillus acidophilus, Lactobacillus brevis, Lactobacillus bulgaricus, Lactobacillus casei, Lactobacillus caucasicus, Lactobacillus fermentum, Lactobacillus helveticus, Lactobacillus lactis, Lactobacillus plantarum, Lactobacillus reuteri, Lactobacillus rhamnosus, Oscillospira guilliermondii, Roseburia cecicola, Roseburia inulinivorans, Ruminococcus flavefaciens, Ruminococcus gnavus, Ruminococcus obeum, Stenotrophomonas nitritireducens, Streptococcus cremoris, Streptococcus faecium, Streptococcus infantis, Streptococcus mutans, Streptococcus thermophilus, Anaerofustis stercorihominis, Anaerostipes hadrus, Anaerotruncus colihominis, Clostridium sporogenes, Clostridium tetani, Coprococcus, Coprococcus eutactus, Eubacterium cylindroides, Eubacterium dolichum, Eubacterium ventriosum, Roseburia faeccis, Roseburia hominis, Roseburia intestinalis, Lacatobacillus bifidus, Lactobacillus johnsonii, Lactobacilli, Acidaminococcus fermentans, Acidaminococcus intestine, Blautia hydrogenotrophica, Citrobacter amalonaticus, Citrobacter freundii, Clostridium aminobutyricum Clostridium bartlettii, Clostridium cochlearium, Clostridium kluyveri, Clostridium limosum, Clostridium malenominatum, Clostridium pasteurianum, Clostridium peptidivorans, Clostridium saccharobutylicum, Clostridium sporosphaeroides, Clostridium sticklandii, Clostridium subterminale, Clostridium symbiosum, Clostridium tetanomorphum, Eubacterium oxidoreducens, Eubacterium pyruvativorans, Methanobrevibacter smithii, Morganella morganii, Peptomphilus asaccharolyticus, and Peptostreptococcus, and any combination thereof. In one embodiment, the composition can further comprise a prebiotic. In one embodiment, the prebiotic can comprise inulin. In one embodiment, the inulin can be present in an amount of at least about 50 mg/ml in the composition. In one embodiment, the capsule can comprise an enteric coating. In one embodiment, the composition can further comprise an enteric coating. In one embodiment, the capsule can be configured for delivery to a small intestine, a large intestine, an ileum, or a combination thereof, of the subject. In one embodiment, the capsule may not substantially release the population of isolated and purified microbes prior to a small intestine or a large intestine of the subject. In one embodiment, the capsule can dissolve at a pH greater than at least about pH 6.5. In one embodiment, the pharmaceutical composition can comprise one or more enteric coatings. In one embodiment, the composition can be stable at 4° C. for 2 weeks or up to 30 days. In one embodiment, the composition can comprise an amount of at least about 10⁵ colony forming units (CFU) of one or more microbes in the population. In one embodiment, the encapsulated mixture can comprise an amount of at least about 0.1% viable microbes. In one embodiment, the subject can be human. In one embodiment, the mixture can be a powder. In one embodiment, the powder can comprise particles with a uniform particle size. In one embodiment, particles in the powder can be non-cohesive.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIG. 1 illustrates an exemplary embodiment of a method for the formulation of a composition for administration.

FIG. 2 illustrates an exemplary embodiment of a bioreactor configuration for growing one or more isolated and purified microbes.

FIG. 3 shows an exemplary process for lyophilizing one or more isolated and purified microbes and including the microbe in a formulation.

FIG. 4 illustrates strain stability at room temperature (RT) and at 4° C.

FIG. 5A illustrates encapsuled formulation stability at room temperature (RT, 20-25° C.) and at 4° C.

FIG. 5B illustrates formulation stability over time.

FIG. 6 illustrates optimal density measurements over time for the successful GMP growth of Akkermansia muciniphila in vegetable infusion.

FIG. 7 illustrates a representative live and dead microbial count that can be used to create a standard curve for other measurements.

FIG. 8 illustrates reproducible measurements from a series of dilutions in determining the linear relationship between OD and CFU for B. longum.

FIG. 9A illustrates the viable bacterial cell counts of B. longum in a 96 well plate as compared before and after lyophilization.

FIG. 9B illustrates a standard curve plotting cycle threshold against microbial concentration dilutions.

FIG. 10 illustrates the measurements of SCFAs for two metabolites, acetate and butyrate across seven strains.

FIG. 11 uses gas chromatography with a flame ionization detector (GC/FID) to relate GC peak area (a.u.) to the microbial concentration (mM).

FIG. 12 illustrates measurements of the metabolic activity of the microbial cells across time in monitoring a high throughput production of the short chain fatty acids using gas chromatography with a flame ionization detector.

FIG. 13 shows a computer control system that can be programmed or otherwise configured to implement methods provided herein.

### DETAILED DESCRIPTION

General

Altering the microbiome to treat various disorders and improve well-being is an area of great interest and inquiry. However, translating discoveries about the microbiome and individual microbes therein into compositions that can be readily administered to a subject can be quite challenging. Producing such compositions requires (1) efficiently growing such microbes and (2) preserving the microbes in shelf-stable form.

Regarding growth, therapeutically relevant isolated and purified microbes may have metabolic requirements that are difficult to replicate in culture, apart from a naturally occurring microbiome. For instance, many of the isolated and purified microbes that reside in the gut may not be easily cultivated outside of the intestinal environment. Some microbes may require specific nutrients. Exposure to atmospheric oxygen must be limited for obligate anaerobes. Further, isolated and purified microbes must be grown in concentrations adequate for formulations with volume constraints (e.g., a capsule or tablet for oral administration).

Moreover, isolated and purified microbes, particularly obligate anaerobes, may be unstable even when lyophilized. Isolated and purified microbes for administration to a subject must be stable such that active or viable cells remain after a period of days, weeks, or months. Such a shelf life permits formulations to be administered to a subject in an effective and shelf-stable form.

The methods and compositions of the present disclosure address an unmet need for stable compositions of isolated and purified microbes, particularly obligate anaerobes, which can be formulated for administration to a subject. The present disclosure provides approaches for producing stable (e.g., oxygen-stable) compositions of obligate anaerobes. The present disclosure provides techniques for producing formulations comprising obligate anaerobes that remain stable over a period of days, weeks, or months, such that the composition can be stored prior to administration. As an example of stability, particularly oxygen-stability, a composition of one or more isolated and purified obligate anaerobes as described herein, may have at least 0.1%, 1%-5%, at least 10%, or at least 20% of the initial active cells/g remaining after storage for 14 days at 4 degrees Celsius (e.g., 2×10⁹ active cells/gram remain after storage (t=14 days) relative to 1×10¹⁰ active cells/gram at encapsulation (t=0)). The present invention provides compositions and approaches for growing oxygen stable obligate anaerobes for use in a variety of formulations, including capsules and tablets for oral administration. Techniques described herein can be used to produce be highly-concentrated compositions (e.g., 1×10⁹ active cells/g or more). These compositions may be dry powders that can be readily formulated into a variety dosage forms. For example, compositions described herein can be formulated into capsules, tablets, or suppositories. As such, one or more dosage forms can be administered to a subject to treat a disorder or as a medical food.

The present disclosure provides compositions and methods that can be used to produce compositions of obligate anaerobic microbes that remain viable over periods 14 days or more, even while exposed to atmospheric oxygen. An additional advantage of the methods and compositions described herein is that lyophilized products are provided in powder form, obviating the need for further processing (e.g., pulverizing).

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

As used in the specification and claims, the singular forms “a”, “an” and “the” can include plural references unless the context clearly dictates otherwise. For example, the term “a sample” can include a plurality of samples, including mixtures thereof.

The terms “microbes” and “microorganisms” can be used interchangeably herein and can refer to bacteria, archaea, eukaryotes (e.g. protozoa, fungi, yeast), and viruses, including bacterial viruses (i.e. phage).

The term “microbiome”, “microbiota”, and “microbial habitat” are used interchangeably herein and can refer to the ecological community of microorganisms that live on or in a subject's body. The microbiome can be comprised of commensal, symbiotic, and/or pathogenic microorganisms. Microbiomes can exist on or in many, if not most parts of the subject. Some non-limiting examples of habitats of microbiome can include: body surfaces, body cavities, body fluids, the gut, the colon, skin surfaces and pores, vaginal cavity, umbilical regions, conjunctival regions, intestinal regions, the stomach, the nasal cavities and passages, the gastrointestinal tract, the urogenital tracts, saliva, mucus, and feces.

The term “prebiotic” as used herein can be a general term to refer to chemicals and or ingredients that can affect the growth and/or activity of microorganisms in a host (e.g. can allow for specific changes in the composition and/or activity in the microbiome). Prebiotics can confer a health benefit on the host. Prebiotics can be selectively fermented, e.g. in the colon. Some non-limiting examples of prebiotics can include: complex carbohydrates, complex sugars, resistant dextrins, resistant starch, amino acids, peptides, nutritional compounds, biotin, polydextrose, oligosaccharides, polysaccharide, fructooligosaccharide (FOS), fructans, soluble fiber, insoluble fiber, fiber, starch, galactooligosaccharides (GOS), inulin, lignin, psyllium, chitin, chitosan, gums (e.g. guar gum), high amylose cornstarch (HAS), cellulose, β-glucans, hemi-celluloses, lactulose, mannooligosaccharides, mannan oligosaccharides (MOS), oligofructose-enriched inulin, oligofructose, oligodextrose, tagatose, trans-galactooligosaccharide, pectin, resistant starch, xylooligosaccharides (XOS), locust bean gum, P-glucan, and methylcellulose. Prebiotics can be found in foods (e.g. acacia gum, guar seeds, brown rice, rice bran, barley hulls, chicory root, Jerusalem artichoke, dandelion greens, garlic, leek, onion, asparagus, wheat bran, oat bran, baked beans, whole wheat flour, banana), and breast milk. Prebiotics can also be administered in other forms (e.g. capsule or dietary supplement).

The term “probiotic” as used herein can mean one or more microorganisms which, when administered appropriately, can confer a health benefit on the host or subject. Some non-limiting examples of probiotics can include: Akkermansia muciniphila, Anaerostipes caccae, Bifidobacterium adolescentis, Bifidobacterium bifidum, Bifidobacterium infantis, Bifidobacterium longum, Butyrivibrio fibrisolvens, Clostridium acetobutylicum, Clostridium aminophilum, Clostridium beijerinckii, Clostridium butyricum, Clostridium colinum, Clostridium coccoides, Clostridium indolis, Clostridium nexile, Clostridium orbiscindens, Clostridium propionicum, Clostridium xylanolyticum, Enterococcus faecium, Eubacterium hallii, Eubacterium rectale, Faecalibacterium prausnitzii, Fibrobacter succinogenes, Lactobacillus acidophilus, Lactobacillus brevis, Lactobacillus bulgaricus, Lactobacillus casei, Lactobacillus caucasicus, Lactobacillus fermentum, Lactobacillus helveticus, Lactobacillus lactis, Lactobacillus plantarum, Lactobacillus reuteri, Lactobacillus rhamnosus, Oscillospira guilliermondii, Roseburia cecicola, Roseburia inulinivorans, Ruminococcus flavefaciens, Ruminococcus gnavus, Ruminococcus obeum, Stenotrophomonas nitritireducens, Streptococcus cremoris, Streptococcus faecium, Streptococcus infantis, Streptococcus mutans, Streptococcus thermophilus, Anaerofustis stercorihominis, Anaerostipes hadrus, Anaerotruncus colihominis, Clostridium sporogenes, Clostridium tetani, Coprococcus, Coprococcus eutactus, Eubacterium cylindroides, Eubacterium dolichum, Eubacterium ventriosum, Roseburia faeccis, Roseburia hominis, Roseburia intestinalis, Lacatobacillus bifidus, Lactobacillus johnsonii, Lactobacilli, Acidaminococcus fermentans, Acidaminococcus intestine, Blautia hydrogenotrophica, Citrobacter amalonaticus, Citrobacter freundii, Clostridium aminobutyricum Clostridium bartlettii, Clostridium cochlearium, Clostridium kluyveri, Clostridium limosum, Clostridium malenominatum, Clostridium pasteurianum, Clostridium peptidivorans, Clostridium saccharobutylicum, Clostridium sporosphaeroides, Clostridium sticklandii, Clostridium subterminale, Clostridium symbiosum, Clostridium tetanomorphum, Eubacterium oxidoreducens, Eubacterium pyruvativorans, Methanobrevibacter smithii, Morganella morganii, Peptomphilus asaccharolyticus, and Peptostreptococcus, and any combination thereof.

In the present disclosure, “oxygen stable” or oxygen stability of one or more microbes may refer to the response of microbes in response to exposure to gaseous or dissolved oxygen. Oxygen stable microbes may remain viable in environments with gaseous or dissolved oxygen. Microbes may be aerobic, anaerobic, or facultative depending on their characteristic mechanisms to produce energy for growth. Aerobes, during metabolism of energy-containing compounds, may need molecular oxygen as a terminal electron acceptor and may not cultivate in their absence. Contrarily, molecular oxygen can be toxic for anaerobes, which may not grow in their presence. As a result, anaerobic microbes may depend on electron acceptors. Anaerobic microbe's fermentative metabolism may allow organic compound reduction to different end products. The end products may comprise alcohols and organic acids (e.g., acetate and butyrate). Oxygen may be a reactive molecule and prefers to be in a reduced state. Oxygen can easily be reduced into incredibly reactive species such as superoxide radicals and hydrogen peroxide. These highly reactive species can be detrimental when reacting with cell lipid membranes and proteins and can result in cell death. While aerobic organisms may comprise the necessary enzymes to remove the reactive oxygen, the enzymes may be in low concentrations or absent in the anaerobic microbes. The enzymes may comprise peroxidases, superoxide dismutases, and catalases. Furthermore, many enzymes suitable for anaerobic microbe may be oxygen sensitive in some instances. The present disclosure provides methods for producing a formulation comprising oxygen stable anaerobes, which can be unstable in the presence of oxygen.

Facultative microbes can selectively choose oxygen as a terminal electron acceptor. Facultative microbes can also metabolize without oxygen through reduction of other compounds. The ability to use oxygen as a terminal electron acceptor can be an efficient mechanism in generating energy.

Another class of microbes may be obligate anaerobic microbe. The stability of obligate anaerobes can attributed to a variety of factors. Compared to aerobic organisms, obligate anaerobes may not produce enough enzymes to detoxify superoxide and hydrogen peroxide in the cells. The enzymes may include catalases, peroxidases, nitrogenase, and superoxide dismutase. Further, since sulfide may be a constituent for some enzymes, oxygen may oxidize the sulfide to disulfide and inactivate the enzymes. Another reason for the general instability in anaerobes can be that they may contain oxygen sensitive enzymes. The enzymes may be metalloenzymes. The proteins may contain at the active site metals such as molybdenum, tungsten, and iron. These metals can be reactive towards oxygen and can destabilize the protein. The present disclosure describes methods for formulating compositions comprising one or more oxygen stable obligate anaerobes.

The terms “determining”, “measuring”, “evaluating”, “assessing,” “assaying,” and “analyzing” can be used interchangeably herein and can to refer to any form of measurement, and include determining if an element is present or not (e.g., detection). These terms can include both quantitative and/or qualitative determinations. Assessing may be relative or absolute. These terms can include use of the algorithms and databases described herein. “Detecting the presence of” can include determining the amount of something present, as well as determining whether it is present or absent. The term “genome assembly algorithm” as used herein, can refer to any method capable of aligning sequencing reads with each other (de novo) or to a reference (re-sequencing) under conditions that a complete sequence of the genome may be determined.

The term “genome” as used herein, can refer to the entirety of an organism's hereditary information that is encoded in its primary DNA sequence. The genome can include the genes and/or the non-coding sequences. For example, the genome may represent a microbial genome. The genetic content of the microbiome can comprise: genomic DNA, RNA, and ribosomal RNA, the epigenome, plasmids, and all other types of genetic information found in the microbes that comprise the microbiome.

“Nucleic acid sequence” and “nucleotide sequence” as used herein can refer to an oligonucleotide or polynucleotide, and fragments or portions thereof, and to DNA or RNA of genomic or synthetic origin which may be single- or double-stranded, and represent the sense or antisense strand. The nucleic acid sequence can be made up of adenine, guanine, cytosine, thymine, and uracil (A, T, C, G, and U) as well as modified versions (e.g. N6-methyladenosine, 5-methylcytosine, etc.).

The terms “homology” and “homologous” as used herein in reference to nucleotide sequences can refer to a degree of complementarity with other nucleotide sequences. There may be partial homology or complete homology (i.e., identity). A nucleotide sequence which is partially complementary, i.e., “substantially homologous,” to a nucleic acid sequence can be one that at least partially inhibits a completely complementary sequence from hybridizing to a target nucleic acid sequence.

The term “sequencing” as used herein can refer to sequencing methods for determining the order of the nucleotide bases—A, T, C, G, and U—in a nucleic acid molecule (e.g., a DNA or RNA nucleic acid molecule.

The term “biochip” or “array” can refer to a solid substrate having a generally planar surface to which an adsorbent can be attached. A surface of the biochip can comprise a plurality of addressable locations, each of which location may have the adsorbent bound there. Biochips can be adapted to engage a probe interface, and therefore, function as probes. Protein biochips can be adapted for the capture of polypeptides and can be comprise surfaces having chromatographic or biospecific adsorbents attached thereto at addressable locations. Microarray chips can be used for DNA and RNA gene expression detection.

The term “barcode” as used herein, can refer to any unique, non-naturally occurring, nucleic acid sequence that may be used to identify the originating genome of a nucleic acid fragment.

The terms “subject,” “individual,” “host,” and “patient” can be used interchangeably herein and refer to any animal subject, including: humans, laboratory animals, livestock, and household pets. The subject can host a variety of microorganisms. The subject can have different microbiomes in various habitats on and in their body. The subject may be diagnosed or suspected of being at high risk for a disease. The subject may have a microbiome state that is contributing to a disease (a dysbiosis). In some cases, the subject is not necessarily diagnosed or suspected of being at high risk for the disease. In some instances a subject may be suffering from an infection or at risk of developing or transmitting to others an infection.

The terms “treatment” or “treating” can be used interchangeably herein. These terms can refer to an approach for obtaining beneficial or desired results including but not limited to a therapeutic benefit and/or a prophylactic benefit. A therapeutic benefit can mean eradication or amelioration of the underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect can include delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The terms “16S”, “16S ribosomal subunit”, and “16S ribosomal RNA (rRNA)” can be used interchangeably herein and can refer to a component of a small subunit (e.g., 30S) of a prokaryotic (e.g., bacteria, archaea) ribosome. The 16S rRNA can be highly conserved evolutionarily among species of microorganisms. Consequently, sequencing of the 16S ribosomal subunit can be used to identify and/or compare microorganisms present in a sample (e.g., a microbiome).

The terms “23S”, “23S ribosomal subunit”, and “23S ribosomal RNA (rRNA)” can be used interchangeably herein and can refer to a component of a large subunit (e.g., 50S) of a prokaryotic (e.g., bacteria, archaea) ribosome. Sequencing of the 23S ribosomal subunit can be used to identify and/or compare microorganisms present in a sample (e.g., a microbiome).

The term “spore” as used herein can refer to a viable cell produced by a microorganism to resist unfavorable conditions such as high temperatures, humidity, and chemical agents. A spore can have thick walls that allow the microorganism to survive harsh conditions for extended periods of time. Under suitable environmental conditions, a spore can germinate to produce a living form of the microorganism that is capable of reproduction and all of the physiological activities of the microorganism.

The term “anaerobe” as used herein can refer to an organism or microbe such as bacteria that can grow and survive in the absence of oxygen. The anaerobe may be unicellular or multicellular. The three categories of anaerobe can comprise obligate anaerobes, aerotolerant organisms, and facultative anaerobes.

Administration of microbial compositions (e.g., probiotics) to a subject (e.g., to the intestinal tract) may provide many therapeutic benefits. The intestinal microbiota may protect against disease by maintaining a healthy gastrointestinal (GI) tract. The microbial strains in probiotics can be found, for example, in the normal (e.g., healthy) intestinal microbiota and can be beneficial in preserving a healthy GI tract. Probiotic therapy can help in the treatment of, for example, diarrheal diseases, intestinal conditions, and clinical symptoms. The treatment can be considered a natural non-invasive method, for example, to treat a disorder and/or subdue pathogens. Probiotic therapy can be administered orally through pharmaceuticals or food. However, the composition of probiotics can be difficult to formulate, stabilize, and administer.

In an aspect, the present disclosure provides methods for formulating a composition for administration to a subject in need thereof. The method may comprise obtaining a mixture that is substantially dry and comprises about 10% or less residual moisture. The mixture can comprise at most about 0%, 0.01%, 0.1%, 1%, 2%, 3%, 4%, 5%, 6%, 7%, 8%, 9%, 10%, 15%, or 20% of residual moisture. The mixture can comprise a population of isolated and purified microbes. The population can comprise one or more obligate anaerobes. The obligate anaerobes can be oxygen-stable. Residual moisture may be measured using a suitable technique. In some instances, tests for residual moisture can meet and may not exceed the limits as approved by the United States Food and Drug Administration set forth in the Code of Federal Regulations, e.g., 21 C.F.R. 610.13 (Provisions related to the purity of biologics). Techniques for measuring residual moisture may include, for example, gravimetric or loss on drying test, the Karl Fischer methodology for moisture determination, thermogravimetry and thermogravimetry/mass spectrometry.

The population may comprise at least 1, 2, 3, 4, 5, 6, 7, 8, 9, 10, 15, 20, 25, 30, 35, or 40 obligate anaerobes. The mixture can also include a pharmaceutically acceptable carrier. The method may also comprise encapsulating the mixture in an enteric-coated capsule for delivery to said subject.

In another aspect, the present disclosure provides methods for formulating a composition for administration to a subject in need. The method may comprise obtaining a dry mixture. The mixture can comprise a population of isolated and purified microbes. The population may comprise one or more obligate anaerobes that are viable under conditions comprising about 15% or greater oxygen by volume. The population may comprise at least 1, 2, 3, 4, 5, 6, 7, 8, 9, 10, 15, 20, 25, 30, 35, or 40 obligate anaerobes. The population may also comprise at least about 0%, 1%, 2%, 3%, 5%, 10%, 15%, 20%, 25%, 30%, 35%, or 40% oxygen by volume. Furthermore, the composition may comprise a pharmaceutically acceptable carrier. The method may then comprise encapsulating the mixture in a capsular tablet, a capsule, or an enteric-coated capsule for delivery to said subject.

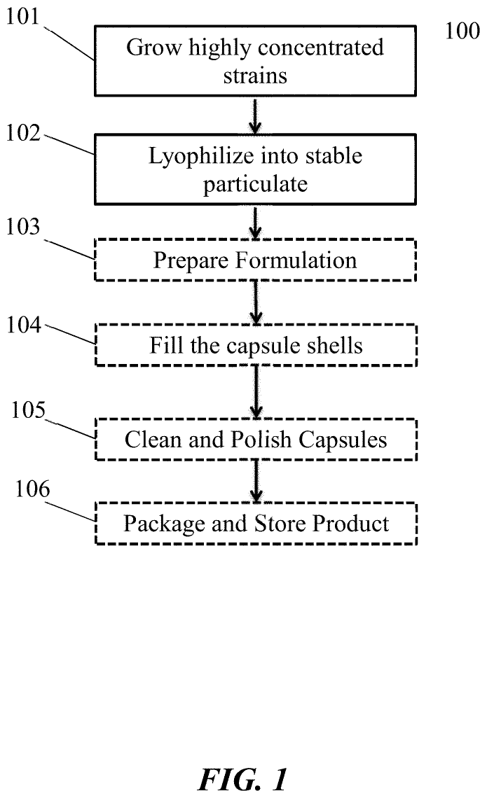

FIG. 1 provides a non-limiting exemplary embodiment of the formulation method. In this example 100, microbes are grown 101 and converted to a shelf-stable composition 102 via lyophilization. Then a formulation is prepared 103 by combining stable particulates of three microbe strains with inulin. Capsule shells are filled 104 with the formulation and the shells are combined to encapsulate the composition. The capsules are cleaned and polished 105. The encapsulated product is stored and packaged 106 for administration to a subject.

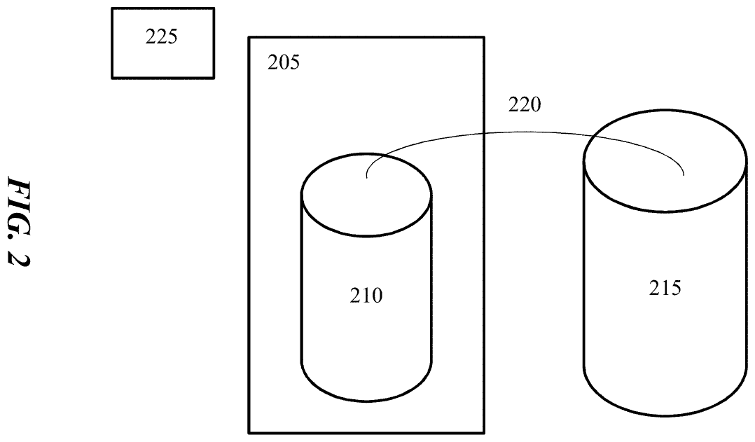

FIG. 2 provides an example of how to grow one or isolated and purified microbes of the present disclosure. FIG. 2 describes a bioreactor 205 comprising a 7 Liter (L) glass tank 210 Initially media 215 is fed into the bioreactor vessel 210. The media is produced in 20 liter (L) batches and is produced in compliance with current good manufacturing practices (cGMP). The media and bioreactor vessel are autoclaved. Inoculant of an isolated and purified microbe 225 (e.g., Bifidobacterium infantis) is added to the vessel 210. The bioreactor is harvested when an optical density (OD) of at least 0.5, 1, 2, 3, or 4 is reached. Per inoculant, the bioreactor is harvested twice. In the first harvest, 50, 55, 60, 65, 70, 75, 80, 85, 90, or 95% % of the culture is harvested by pumping the culture from the bioreactor. The bioreactor is re-fed 220 with media 215 immediately after the first volume of culture is harvested. The bioreactor ferments the second batch of the inoculant 225 similarly to the first batch. Microbe growth is in complicance with current good manufacturing practices (cGMP).

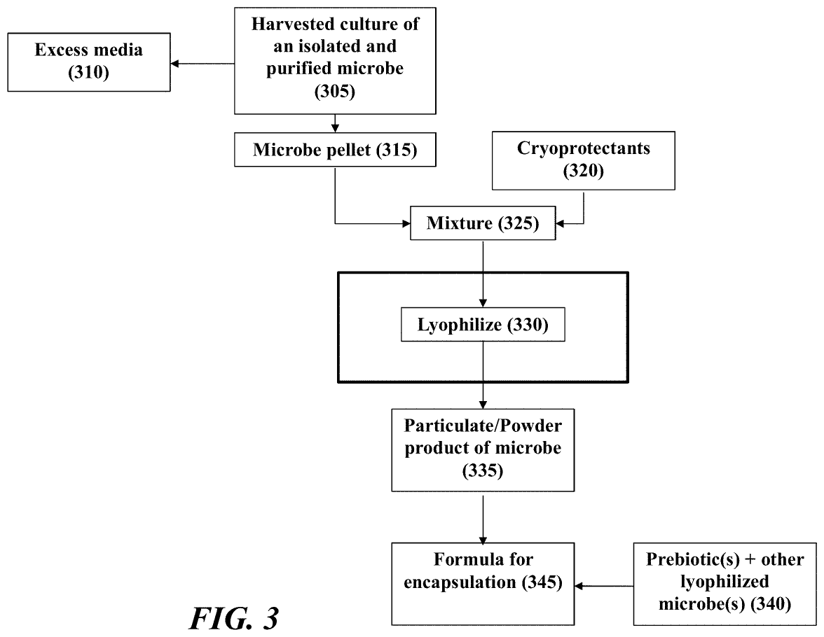

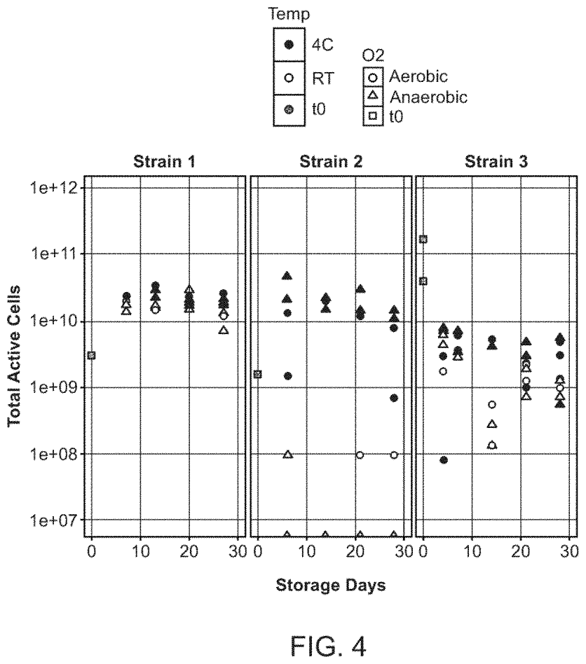

Once grown, the one or more microbes are lyophilized to yield a stable particulate composition 102. As shown in FIG. 3, the harvested product is first centrifuged. Excess media 310 is discarded and a pellet 315 of the isolated and purified microbes is retained. Microbe-specific cryoprotectants 320 are added to the pelleted microbes to form a cryoprotected mixture 325. The cyproprotectants 320 are cGMP-compliant. The mixture 325 is spread into metal trays for lyophilization 330. The product 335 is a particulate that is beige to dark tan in color. The particulate particles can also be characterized as a powder. In some instances, the composition does not require further processing (e.g. pulverizing or grinding) before being incorporated into capsules, tablets, suppositories or other dasage forms suitable for administration to a subject. However, the particulate product can optionally be ground or pulverized to achieve size uniformity. Particulates of individual isolated and purified microbe strains may be stable at room temperature or 4 degrees Celisus (FIG. 4). Room temperature is 20 to 25 degrees Celsius.

Optionally, the formulation 103 is further prepared by mixing the lyophilized microbes with excipients and/or prebiotics. As shown in FIG. 3, in one example 345, a population of isolated and purified microbes each separately lyophilized, are combined with a prebiotic, such as inulin (340). Each of the isolated and purified microbes and prebiotic are added in approximately equal portions, by weight.

The composition is filled into capsule shells 104 and the capsules are closed to yield an encapsulated formulation. The capsule shells can be pH sensitive/insensitive or specially coated to release in specific parts of the GI tract. Each capsule contains 1×10⁸ to 1×10¹⁰ active cells. Each capsule shell contains a proportionate amount of each microbe and prebiotic relative to the formula for encapsulation 345. The capsules are cleaned and polished to remove any debris 105.

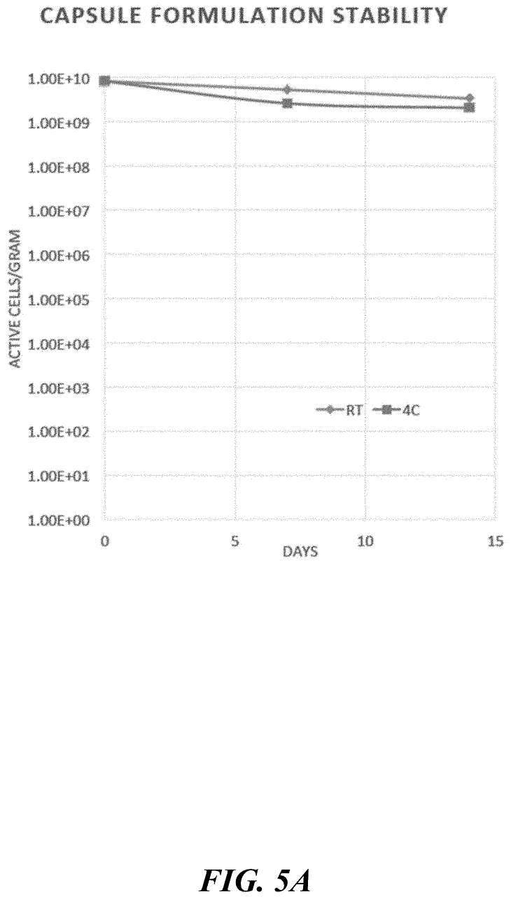

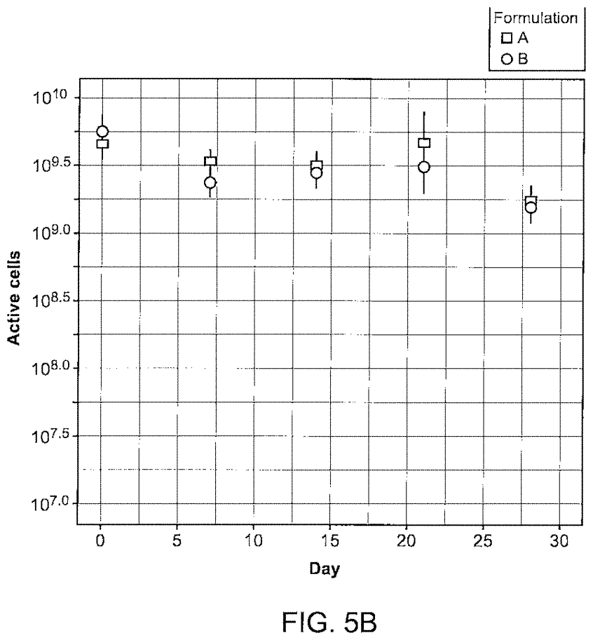

The polished capsules are packaged and stored 106 in safety-sealed plastic that may be from Bel-Art, Biorx, ColorSafe, CSP Vials, Dynalon, MP Vials, PSA, Pill Pod, Qorpak, Safer Lock, or Wheaton. Formulations maintain stability for 2 weeks or more when stored at 4 degrees Celsius and at room temperature (20 to 25 degrees Celsius) (FIG. 5A and FIG. 5B).

### Probiotic Strains of Bacteria

A composition may comprise an isolated and purified microbe.

The microbe may be a fermenter. Fermentation microbes (e.g., fermenters) may be anaerobic and can utilize organic molecules as their final electron acceptor produce the final products of fermentation. Fermenters can metabolize some sugars and their analogues as specific to specific microbes. The products of fermenters can comprise propionic acids (e.g., indole-3-propionate) and short fatty chain acids. Short fatty chain acids may include, but are not limited to: formate, acetate, propionate (e.g., indole-3-propionate), butyrate, isobutyrate, valerate, and isovalerate. The composition may comprise at least one primary fermenter. Additionally, the therapeutic composition can comprise at least one primary fermenter and at least one secondary fermenter. A therapeutic composition can comprise at least one primary fermenter, at least one secondary fermenter, and at least one prebiotic.

The microbe may be selected from a phylum, class, order, family, genus, species, and clostridial cluster. The phylum can be selected from the group consisting of Actinobacteria, Bacteroidetes, Cyanobacteria, Firmicutes, Fusobacteria, Proteobacteria, Spirochaetes, Tenericutes, Verrucomicrobia. The class may be selected from the group consisting of Verrucomicrobiae, Clostridia, or Actinobacteria. The order can be selected from the group consisting of Verrucomicrobiales, Clostridiales, Bificobacteriales, or Clostridiales. The family may be selected from the group consisting of Alcaligenaceae, Bifidobacteriaceae, Bacteroidaceae, Clostridiaceae, Coriobacteriaceae, Enterobacteriaceae, Enterococcaceae, Erysipelotricaceae, Eubacteriaceae, Incertae-Cedis-XIII, Incertae-Sedis-XIV, Lachnospiraceae, Lactobacillaceae, Pasturellaceae, Peptostreptococcaceae, Porphyromonadaceae, Prevotellaceae, Rikenellaceae, Ruminococcaceae, Streptococcaceae, Veillonellaceae, Verrucomicrobiaceae. The genus can be selected from the group consisting of Akkermansia, Clostridium, Eubacterium, Bifidobacterium, or Faecalibacterium. The clostridial cluster may be selected from the group consisting of Cluster I, Cluster XIVA, or Cluster IV.

In one non-limiting example, a therapeutic composition can comprise Bifidobacterium adolescentis, Clostridium indolis, and inulin. In another non-limiting example, a therapeutic composition can comprise Bifidobacterium longum, Faecalibacterium prausnitzii, and starch. In yet another non-limiting example, a composition may comprise Akkermansia muciniphila, Clostridium beijerinckii, Clostridium butyricum, Eubacterium hallii, and inulin. In still another non-limiting example, the composition may comprise Akkermansia muciniphila, Bifidobacterium adolescentis, Bifidobacterium infantis, Bifidobacterium longum, Clostridium beijerinckii, Clostridium butyricum, Clostridium indolis, Eubacterium hallii, and a prebiotic. In still another non-limiting example, the composition may comprise Akkermansia muciniphila, Bifidobacterium infantis, Clostridium beijerinckii, Clostridium butyricum, Eubacterium hallii, and a prebiotic. In still another non-limiting example, the composition may comprise Clostridium beijerinckii, Clostridium butyricum, Bifidobacterium infantis, and a prebiotic. In still another non-limiting example, the composition may comprise Akkermansia muciniphila, Clostridium beijerinckii, Clostridium butyricum, Eubacterium hallii, Bifidobacterium infantis, and a prebiotic. In an additional example, the composition may comprise 1, 2, 3, 4, 5, or more of Akkermansia muciniphila, Bifidobacterium adolescentis, Bifidobacterium infantis, Bifidobacterium longum, Clostridium beijerinckii, Clostridium butyricum, Clostridium indolis, Eubacterium hallii, and Faecalibacterium prausnitzii and a prebiotic. In an yet another example, the composition may comprise 1, 2, 3, 4, 5, or more of Akkermansia muciniphila, Bifidobacterium adolescentis, Bifidobacterium infantis, Bifidobacterium longum, Clostridium beijerinckii, Clostridium butyricum, Clostridium indolis, Eubacterium hallii, and Faecalibacterium prausnitzii and inulin.

Akkermansia muciniphila can be a gram negative, strict anaerobe that can play a role in mucin degradation. Levels of Akkermansia muciniphila can be reduced in subjects with metabolic disorders, for example, obesity and T2DM. Akkermansia muciniphila can protect against metabolic disorder, for example, through increased levels of endocannabinoids that control inflammation, the gut barrier, and gut peptide secretion.

Bifidobacterium adolescentis can be a gram-positive anaerobe, which can be found in healthy human gut from infancy. Bifidobacterium adolescentis can synthesize B vitamins. Bifidobacterium adolescentis can serve as a primary fermenter.

Bifidobacterium infantis can be a gram-positive, catalase negative, micro-aerotolerant anaerobe. Bifidobacterium infantis can serve as a primary fermenter.

Bifidobacterium longum can be a gram-positive, catalase negative, micro-aerotolerant anaerobe. Bifidobacterium longum can serve as a primary fermenter.

Clostridium beijerinckii can be a gram-positive, strict anaerobe that belongs to Clostridial cluster I. Clostridium beijerinckii can serve as a secondary fermenter.

Clostridium butyricum can be a gram-positive, strict anaerobe that can serve as a secondary fermenter.

Clostridium indolis can be a gram-positive, strict anaerobe that belongs to Clostridial cluster XIVA. Clostridium indolis can serve as a secondary fermenter.

Eubacterium hallii can be a gram-positive, anaerobe that belongs to Arrangement A Clostridial cluster XIVA. Eubacterium hallii can serve as a secondary fermenter.

Faecalibacterium prausnitzii can be a gram-positive, anaerobe belonging to Clostridial cluster IV. Faecalibacterium prausnitzii can be one of the most common gut bacteria and the largest butyrate producer. Faecalibacterium prausnitzii can serve as a secondary fermenter.

Clostridium sporogenes can produce or be involved in the production of a short chain fatty acid such as indole-3-propionate.

Measuring the microbiome of hosts can show that microbiomes lacking various strains of microorganisms can result in a health condition and/or disease state (e.g. T2DM and obesity). Restoring one or more lacking strains (e.g. via a bacterial strain such as E. hallii or treatment with fermented milk products) can result in alteration of the health condition. Some non-limiting examples include altering the gut microbiome such that the host has an increased capacity for energy harvest, increased insulin sensitivity, and/or decreased appetite.

A composition can be formulated such that the population comprises one or more isolated and purified microbes selected from the group consisting of: Akkermansia muciniphila, Anaerostipes caccae, Bifidobacterium adolescentis, Bifidobacterium bifidum, Bifidobacterium infantis, Bifidobacterium longum, Butyrivibrio fibrisolvens, Clostridium acetobutylicum, Clostridium aminophilum, Clostridium beijerinckii, Clostridium butyricum, Clostridium colinum, Clostridium coccoides, Clostridium indolis, Clostridium nexile, Clostridium orbiscindens, Clostridium propionicum, Clostridium xylanolyticum, Enterococcus faecium, Eubacterium hallii, Eubacterium rectale, Faecalibacterium prausnitzii, Fibrobacter succinogenes, Lactobacillus acidophilus, Lactobacillus brevis, Lactobacillus bulgaricus, Lactobacillus casei, Lactobacillus caucasicus, Lactobacillus fermentum, Lactobacillus helveticus, Lactobacillus lactis, Lactobacillus plantarum, Lactobacillus reuteri, Lactobacillus rhamnosus, Oscillospira guilliermondii, Roseburia cecicola, Roseburia inulinivorans, Ruminococcus flavefaciens, Ruminococcus gnavus, Ruminococcus obeum, Stenotrophomonas nitritireducens, Streptococcus cremoris, Streptococcus faecium, Streptococcus infantis, Streptococcus mutans, Streptococcus thermophilus, Anaerofustis stercorihominis, Anaerostipes hadrus, Anaerotruncus colihominis, Clostridium sporogenes, Clostridium tetani, Coprococcus, Coprococcus eutactus, Eubacterium cylindroides, Eubacterium dolichum, Eubacterium ventriosum, Roseburia faeccis, Roseburia hominis, Roseburia intestinalis, Lacatobacillus bifidus, Lactobacillus johnsonii, Lactobacilli, Acidaminococcus fermentans, Acidaminococcus intestine, Blautia hydrogenotrophica, Citrobacter amalonaticus, Citrobacter freundii, Clostridium aminobutyricum Clostridium bartlettii, Clostridium cochlearium, Clostridium kluyveri, Clostridium limosum, Clostridium malenominatum, Clostridium pasteurianum, Clostridium peptidivorans, Clostridium saccharobutylicum, Clostridium sporosphaeroides, Clostridium sticklandii, Clostridium subterminale, Clostridium symbiosum, Clostridium tetanomorphum, Eubacterium oxidoreducens, Eubacterium pyruvativorans, Methanobrevibacter smithii, Morganella morganii, Peptomphilus asaccharolyticus, and Peptostreptococcus, and any combination thereof.

The composition may comprise isolated and purified Akkermansia muciniphila, Bifidobacterium infantis, Clostridium beijerinckii, Clostridium butyricum, and Eubacterium hallii. The composition may comprise isolated and purified Clostridium beijerinckii, Clostridium butyricum, and Bifidobacterium infantis.

The composition may comprise isolated and purified Bifidobacterium adolescentis, Akkermansia muciniphila, Eubacterium hallii, and Clostridium indolis. The composition may comprise two or more isolated and purified microbes selected from the group consisting of: Bifidobacterium adolescentis, Akkermansia muciniphila, Eubacterium hallii, and Clostridium indolis.

The composition may comprise isolated and purified Akkermansia muciniphila, Clostridium beijerinckii, Clostridium butyricum, Eubacterium hallii, and Bifidobacterium infantis.

The composition may comprise isolated and purified Bifidobacterium infantis, Bifidobacterium longum, Clostridium beijerinckii, Clostridium butyricum, and Eubacterium hallii. The composition may comprise two or more isolated and purified microbes selected from the group consisting of: Bifidobacterium infantis, Bifidobacterium longum, Clostridium beijerinckii, Clostridium butyricum, and Eubacterium hallii.

The composition may comprise isolated and purified Clostridium indolis, Bifidobacterium longum, and Akkermansia muciniphila.

The composition may comprise isolated and purified Bifidobacterium bifidum and Lactobacillus brevis.

Examples of obligately anaerobic bacterial genera may include Akkermansia, Actinomyces, Bacteroides, Bifidobacterium, Clostridium, Eubacterium, Faecalibacterium, Fusobacterium, Peptostreptococcus, Porphyromonas, Prevotella, Propionibacterium, and Veillonella. Clostridium species can be an endospore-forming bacteria, and can survive in atmospheric concentrations of oxygen in this dormant form.

### Growth of Highly Concentrated Strains

Microbes formulated in a composition comprising a population of isolated and purified microbes comprising one or more obligate anaerobes may be grown using a variety of techniques. These techniques may be directed to growing or culturing anaerobic bacteria.

Microbes can be produced in any suitable medium for growth, some non-limiting examples of medium include: RCM (Reinforced Clostridial Medium), nutrient media, minimal media, selective media, differential media, and transport media.

A sample growth media recipe may comprise elements such as animal and/or vegetable based—peptones, amino acids, extracts, carbon and energy sources, hydrolysates, infusions, and yeast extracts, soya peptones, lactalbumins, bile salts & derivatives, sugars, HiVeg hydrolysates, HiVeg extract, yeast extract, sodium thioglycolate, oxidation reduction indicators, vitamins, salt, calcium carbonate, antifoam, buffering agents, surfactants, reducing agents, phenol red, sodium pyruvate, glutathione, hypoxanthine.Na, thymidine, lipoic acid, linoleic acid, putrescine 2HCl, bactopeptone, thymine, adenine sulphate, adenosine-5-triphosphate, cholesterol, 2-deoxy-D-ribose, guanine HCl, sodium acetate, uracil, xanthine Na, cysteine HCl, water, and agar. A growth media may be a solid or liquid. A solid growth media may comprise a silica gel, pectin, gelatin, and agar.

Carbon and energy sources may comprise glucose, starch, sodium acetate, sodium citrate, and oxaloacetate. Sugars can comprise sucrose, glucose, lactose, galactose, dextrose, maltose, xylose, ribose, sorbitol, N-Acetylglucosamine, and mannitol. Oxidation reduction indicators can include methylene blue, resazurin, indigo carmine, 5,5′, 7-indigo trisulfonic acid, tetrapotassium salt, 2,6-dichloroindophenol sodium salt hydrate, methyl viologen dichloride, resorufin sodium salt, and phenosafranine. Buffering agents can comprise cacodylates, citrates, phosphates, glycine, tris, acetates, borates, and carbonates such as sodium bicarbonate, 4-(2-hydroxyethyl)-1-piperazineethanesulfonic acid (HEPES), N-(2-acetamido)-aminoethanesulfonic acid (ACES), N-(2-acetamido)-iminodiacetic acid, 2-aminoethanesulfonic acid, ammonia, 2-amino-2-methyl-1-propanol (AMP), 2-amino-2-methyl-1,3-propanediol (AMPSO), ammediol, N-(1,1-dimethyl-2-hydroxyethyl)-3-amino-2-hydroxypropanesulfonic acid (AMPSO), N,N-bis(2-hydroxyethyl)-glycine (bicine), [bis-(2-hydroxyethyl)-imino]-tris-(hydroxymethylmethane) (Bis-Tris), 1,3-bis[tris(hydroxymethyl)-methylaminol]propane (Bis-Tris-Propane), boric acid, cacodylate, 3-(cyclohexylamino)-propanesulfonic acid (CAPS), 3-(cyclohexylamino)-2-hydroxyl-1-propanesulfonic acid (CAPSO), cyclohexylaminoethanesulfonic acid (CHES), 3-[N-bis(hydroxyethyl)amino]-2-hydroxypropanesulfonic acid (DIPSO), and potassium hydrogen phosphate.

Surfactants may comprise polyoxyethylene glycol octylphenol ethers, fatty alcohols, and polyoxyethylene glycol sorbitan alkyl esters. The surfactant may also be a non-ionic surfactant. The surfactant can be selected from the group consisting of Tween 80, polysorbate 20 (PS20), and poloxamer 188 (P188). Amino acids may comprise L-arginine, L-cysteine, L-cysteine, L-histidine, L-isoleucine, L-leucine, L-lysine, L-methionine, L-phenylalanine, L-threomine, L-typtophan, L-tyrosine, L-valine, L-alanine, L-asparagine, L-aspartic acid, L-glutamic acid, L-glutamine, glycine, L-proline, L-serine, and L-hydroxyproline. Peptones can include soya peptone, HiVeg Peptone #1, HiVeg Peptone #2, HiVeg Peptone #3, HiVeg Peptone #4, HiVeg Peptone #5, HiVeg Special Peptone, protease peptone.

Other elements may comprise HiVeg Special Infusion, HiVeg Extract #2, Cystein-HCl, and Antifoam B silicone Emulsion. The Antifoam B silicone Emulsion may be at least about 20 microliters per liter (μL/L), 25 μL/L, 30 μL/L, 35 μL/L, 40 μL/L, 45 μL/L, 50 μL/L, 55 μL/L, 60 μL/L, 65 μL/L, or 70 μL/L broth.

The amounts of elements added to the growth media may be at least about 0.01 g/L, 0.02 g/L, 0.03 g/L, 0.04 g/L, 0.05 g/L, 0.06 g/L, 0.07 g/L, 0.08 g/L, 0.09 g/L, 0.1 g/L, 0.2 g/L, 0.3 g/L, 0.4 g/L, 0.5 g/L, 0.6 g/L, 0.7 g/L, 0.8 g/L, 0.9 g/L, 1 g/L, 2 g/L, 3 g/L, 4 g/L, 5 g/L, 6 g/L, 7 g/L, 8 g/L, 9 g/L, 10 g/L, 15 g/L, 20 g/L, 25 g/L, or 30 g/L. The amounts of elements added to the growth media may be at least about 0.01 mL/L, 0.02 mL/L, 0.03 mL/L, 0.04 mL/L, 0.05 mL/L, 0.06 mL/L, 0.07 mL/L, 0.08 mL/L, 0.09 mL/L, 0.1 mL/L, 0.5 mL/L, 1 mL/L, 5 mL/L, 10 mL/L, 15 mL/L, 20 mL/L, 25 mL/L, 30 mL/L, 35 mL/L, 40 mL/L, 45 mL/L, 50 mL/L, 100 mL/L, 200 mL/L, 300 mL/L, 400 mL/L, 500 mL/L, 600 mL/L, 700 mL/L, 800 mL/L, 900 mL/L, 1000 mL/L, or 1500 mL/L.

The growth medium may have a varying level of acidity. The preferred pH of growth media may depend upon the optimal growth environment for cultured microbes, e.g., acidophiles. The pH of a growth medium can be, for example, about 7. The pH of a growth medium can be, for example, about 3, about, 4, about 5, about 6, about 7, or about 8.

To grow and culture microbes formulated in a composition comprising one or more obligate anaerobes, the growth medium can have additional attributes that may be advantageous to formulating a composition comprising one or more microbes. For instance, the growth medium can improve the maximum density to which a microbial strain can grow. The growth medium can allow for higher strain concentrations. The growth medium can buffer acid production by a microbial strain, which can minimize the inhibitory effect of, for example, very low pH.

Trace minerals may include aluminum potassium sulfate anhydrous, calcium chloride anhydrous, cobalt (II) chloride, copper (II) chloride dihydrate, copper (II) sulfate pentahydrate, cobalt (II) nitrate dihydrate, cobalt (II) nitrate hexahydrate, boric acid, iron (II) sulfate pentahydrate, iron (II) sulfate heptahydrate, manganese (II) chloride dihydrate, manganese (II) chloride tetrahydrate, manganese (II) chloride heptahydrate, manganese (II) sulfate monohydrate, manganese (II) sulfate dihydrate, manganese (II) sulfate tetrahydrate, manganese (II) sulfate heptahydrate, magnesium sulfate monohydrate, magnesium sulfate pentahydrate, magnesium sulfate heptahydrate, magnesium sulfate nonahydrate, disodium ethylenediaminetetraacetic acid, sodium molybdate dehydrate, sodium chloride, sodium selenite, sodium tungstate dihydrate, nickel (II) chloride, nickel (II) chloride hexahydrate, zinc sulfate monohydrate, and zinc sulfate heptahydrate. The trace minerals added to the growth media may be at least about 0.1 mg/L, 0.2 mg/L, 0.3 mg/L, 0.4 mg/L, 0.5 mg/L, 0.6 mg/L, 0.7 mg/L, 0.8 mg/L, 0.9 mg/L, 1 mg/L, 1 mg/L, 2 mg/L, 3 mg/L, 4 mg/L, 5 mg/L, 6 mg/L, 7 mg/L, 8 mg/L, 9 mg/L, 10 mg/L, 11 mg/L, 12 mg/L, 13 mg/L, 14 mg/L, 15 mg/L, 16 mg/L, 17 mg/L, 18 mg/L, 19 mg/L, 20 mg/L, 21 mg/L, 22 mg/L, 23 mg/L, 24 mg/L, 25 mg/L, 26 mg/L, 27 mg/L, 28 mg/L, 29 mg/L, 30 mg/L, 35 mg/L, 40 mg/L, 45 mg/L, or 50 mg/L.

Vitamins may include D-biotin, Ca-pantothenate, myinositol, p-aminobenzoic acid, folic acid, pyridoxine hydrochloride, pyridoxine (B6), biotin, riboflavin, lipoic acid, thiamine dichloride, mercaptoethane sulfonic acid, nicotinic acid, pantothenic acid, vitamin A, vitamin B12, vitamin K, riboflavin (B2), thiamine (B1), K-Ca-pantothenate, choline chloride, i-inositol, niacinamide, pyridoxal HCl, pyridoxine HCl, thiamine HCl, para-aminobenzoic acid, niacin, ascorbic acid, a-Tocopherol phosphate, calciferol, menadione, and nicotinic acid. The vitamins added to the growth media may be at least about 0.01 milligrams per liter (mg/L), 0.02 mg/L, 0.03 mg/L, 0.04 mg/L, 0.05 mg/L, 0.06 mg/L, 0.07 mg/L, 0.08 mg/L, 0.09 mg/L, 0.1 mg/L, 0.2 mg/L, 0.3 mg/L, 0.4 mg/L, 0.5 mg/L, 0.6 mg/L, 0.7 mg/L, 0.8 mg/L, 0.9 mg/L, 1 mg/L, 1 mg/L, 2 mg/L, 3 mg/L, 4 mg/L, 5 mg/L, 6 mg/L, 7 mg/L, 8 mg/L, 9 mg/L, 10 mg/L, 11 mg/L, 12 mg/L, 13 mg/L, 14 mg/L, 15 mg/L, 16 mg/L, 17 mg/L, 18 mg/L, 19 mg/L, 20 mg/L, 21 mg/L, 22 mg/L, 23 mg/L, 24 mg/L, 25 mg/L, 26 mg/L, 27 mg/L, 28 mg/L, 29 mg/L, 30 mg/L, 35 mg/L, 40 mg/L, 45 mg/L, or 50 mg/L. The vitamin mix may be at least about 50×, 55×, 60×, 65×, 70×, 75×, 80×, 85×, 90×, 95×, 100×, 150×, or 200×.

Salts may include ammonium chloride, calcium chloride, calcium chloride dihydrate, calcium chloride hexahydrate, calcium chloride decahydrate, ferric nitrate, magnesium sulfate monohydrate, magnesium sulfate pentahydrate, magnesium sulfate heptahydrate, magnesium chloride, magnesium sulfate, magnesium sulfate nonahydrate, meridianiite, magnesium sulfate dodecahydrate, potassium chloride, potassium hydrogen phosphate, potassium dihydrogen phosphate, monopotassium phosphate, potassium sulfate, sodium hydrogen carbonate, sodium hydrogen phosphate, and sodium chloride. The salts added to the growth media may be at least about 0.01 mg/L, 0.02 mg/L, 0.03 mg/L, 0.04 mg/L, 0.05 mg/L, 0.06 mg/L, 0.07 mg/L, 0.08 mg/L, 0.09 mg/L, 0.1 mg/L, 0.2 mg/L, 0.3 mg/L, 0.4 mg/L, 0.5 mg/L, 0.6 mg/L, 0.7 mg/L, 0.8 mg/L, 0.9 mg/L, 1 mg/L, 1 mg/L, 2 mg/L, 3 mg/L, 4 mg/L, or 5 mg/L. The salt solution may be 5×, 10×, 15×, 20×, 25×, 30×, 35×, or 40×.

A growth medium can comprise PYGveg, vitamins, salt, and a buffer.

Sources of nutrition used during microbial culture may comprise nitrogen sources, carbon sources, growth factors, trace elements, inducers, repressors, precursors, antifoams, and water. Nitrogen sources can be selected from the group consisting of corn steep liquor, slaughterhouse wastes, urea, ammonium salts, nitrate, peanut granules, soyabean meal, soya meal, yeast extract, and distilled solubles. Carbon sources may include corn steep liquor, slaughterhouse wastes, urea, ammonium salts, nitrate, peanut granules, soyabean meal, soya meal, yeast extract, and distilled solubles. Bacteria may also require trace elements that may be associated with stimulation of metabolism or enzymes and proteins. The elements may include zinc, manganese, molybdenum, iron, copper, and cobalt. Catabolic enzymes may be used in the presence of inducers. For example, the inducer may be yeast extract. The catabolic enzymes may be repressed by other compounds in the culture medium.

A computational model can be used to predict an optimal growth media. The computation model can use multinomial regression. The computational model can use flux balance analysis (FBA). The optimal growth media can be a growth media determined to yield an optimal growth rate of at least one microbe in the composition. The optimal growth media can be selected from growth medias such as, for example, Autoinducer Bioassay (AB) minimal media, Davis Mingioli (DM) media, and Bochner defined minimal media. The optimal growth media can comprise at least one component determined to induce growth of at least one microbe in the composition. In some cases, metabolic reconstruction is performed of at least one microbe in the composition to determine a target metabolic pathway. Metabolic reconstruction can include determination of at least one enzyme related to growth in the genome of the at least one microbe. Quantities of electron donors and electron acceptors of the target metabolic pathway can be determined. Determination of the quantities of the electron donors and electron acceptors of the target metabolic pathway can determine the at least one component of the growth media to induce growth of the at least one microbe.

Determining growth of isolated and purified microorganisms for formulating in a composition for administration to a subject in need thereof can be accomplished in a variety of different ways. Microbial growth can be assessed through detection methods such as staining, liquid agar culture media, auto-fluorescence micro-colony detection, or electron microscopy.

One or more methods may be used in determining the microbial cell number. The methods may be selected from molecular viability testing, polymerase chain reaction (PCR), reverse transcriptase PCT (RT-PCR), real time-quantitative polymerase chain reaction, ethidium monoazide-PCR, propidium monoazide PCR, fluorescent activated cell sorting, total population count, viable counts, plate count, and turbidity measurements. Total population count may be measured using direct or indirect methods. Direct methods during phase contrast microscopy may comprise direct observation of cells in a specialized counting chamber slide. The value can be expressed as total bacterial per millimeter of bacteria in the sample dead or alive. Cell count in liquid media can be expressed as a concentration of number of cells per unit volume. In contrast, indirect methods may include impedance microbiological techniques and turbidity measurements of a culture using a spectrophotometer or qualitatively against a turbidity standard. The standard may be a McFarland standard. Since cells can absorb and scatter light, cell concentration may be directly proportional to the turbidity. Spectrophotometers can detect the light intensity. When the cell culture is placed in a transparent cuvette, the absorption is measured against the medium. Optical density (OD) measurements may be collected and can be directly proportional to the biomass in the cell suspension in a given range specific to cell type.

In some cases, a counting chamber may be used for counting. A counting chamber may comprise a microscopic slide with a sink in the middle for receiving a drop of the cell culture.

Determination of the number of live bacteria in a sample can be carried out using a viable counts method. Determining the viable counts may include measuring the growth rates and determining disinfectant effectiveness through serial dilution of microbial samples. After the serial dilutions, the microbial samples may be plated on suitable growth media. The samples may further be filtered through a membrane on a growth media soaked pad. The plates can be incubated for at least about 10 hours, 15 hours, 20 hours, 25 hours, or 30 hours until viable colonies appear. The growth of colonies on the plate may be originating from one viable microbial unit. The colonies may also have originated from a group of cells or a single cell. The products are reported as colony forming units (CFUs). Each cell may be a single colony or a CFU. After the colonies are counted and from the culture spread on the plate, the cell concentration may be calculated.

Methods for counting microbes may be selected from a group consisting of overlay plate, pour plate, and surface count. During overlay and pour methods, molten agar may be used to suspend the microbial sample. The colonies can remain small and compact. Higher concentration plates can be counted because the colonies are distinct and not touching one another. The surface count method may also be used and can provide accurate and reliable results. The process involves diluting the bacterial culture with turbidity, pipetting a small volume of bacteria onto the plate surface and spreading evenly on the surface.

In some cases, cell counting may be automated through methods such as electrical resistance, flow cytometry, and image analysis. During flow cytometry, cells may travel in a narrow stream before a laser beam. As each cell is excited by the laser beam, a light detector identifies the light that is reflected in the cells. In addition to quantification, flow cytometers may also detect cell shape and quantify the protein and other biochemical markers in the cells.

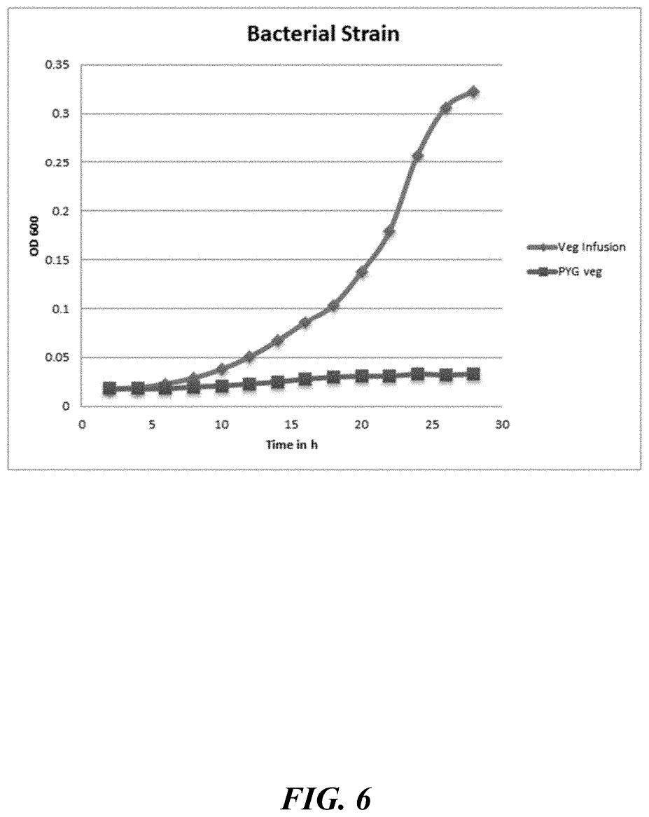

FIG. 6 shows the optical density measurements over time for the growth of Akkermansia muciniphila in vegetable-based growth media (Vegetable infusion and PYG Veg) under Good Manufacturing Practice (GMP) conditions.

In some cases, the concentration of microbes in culture may be increased by extending the log phase of microbial growth. This may be achieved by using the growth curves of microbes and measuring the OD of the microbes in culture. The OD of the microbes can be correlated with microbial growth curve to detect the different growth phases of the microbes. As the microbes are exiting out of the log phase to enter the stationary phase, additional nutrients may be added to the culture. These additional nutrients may lead to a second growth phase thus enhancing the growth of microbes in culture. In some embodiments, the nutrients may include sugars or carbohydrates.

In some cases, the microbes may be grown in a first set of nutrients and as the microbes are exiting the log phase, an additional quantity of the first set of nutrients may be provided in culture to enhance the growth of microorganisms. In some embodiments, the additional quantity of the nutrients added is 5% of the initial quantity. In some embodiments, the additional quantity may be up to 1%, 2%, 5%, 10%, 20%, 30%, or 50% of the initial quantity.

In some embodiments, a second set of nutrients or nutrient source may be provided as the microbes are exiting the log phase.

In some embodiments, a mixture of an additional quantity of the first set of nutrients and a second set of nutrients may be provided as the microbes exit the log phase. In some cases, the additional quantity of the first set of nutrients may be up to 1%, 2%, 5%, 10%, 20%, 30%, or 50% of the initial quantity.

In some cases, the microbes may be harvested as they are exiting the second growth phase. Alternatively, the microbes may be harvested before the second growth phase is complete.

Microbial growth can be assessed through sequencing. In some instances, a plurality of strain-specific sequences can be determined from genomic sequences of each of at least microbes. The at least microbes can be from at least two microbes in the composition. The at least two microbes can be at least two microbes in the gut microbiome from an individual. Target primer pairs can be designed for the plurality of strain-specific sequences. In some cases, quantitative polymerase chain reaction (qPCR) is done in parallel using the target primer pairs to sequence the plurality of strain-specific sequences. The qPCR can produce sequencing data. In some cases, qPCR comprises the use of a nano-well array. The nano-well array can be any suitable nano-well array, such as a SmartChip™ (WafterGen). The sequencing data can be used to determine the growth rate of each of the microbes in the composition. The sequencing data can be used to calculate the ratio of DNA from the origin of replication to the terminus of replication for each of the plurality of strain specific sequences. The ratio of DNA from the origin of replication to the terminus of replication can be proportional to growth rate as a function of cellular replication. In some cases, the quanitity or the growth of microbes in the gut of an individual is determined in situ.

Transformation of Strains into Oxygen-Stable Microbes

Once grown, the bacterial strains may be lyophilized or freeze dried. During lyophilization (also referred to as freeze drying), sublimation may occur and the liquid may be removed as water vapor. Lyophilization can be used to preserve microbial cultures and minimize the damage caused by strictly drying the sample. Lyophilization can also promote high cell viability and metabolic activity when administered in a composition. Lyophilization can be a suitable dehydration process for bacteria to obtain a solid and stable final formulation. Without protection afforded by preservation via lyophilization, cells may die and those that survive can die rapidly after storage. The choice of an appropriate cryoprotectant and drying medium mixture may be important to increase the survival rate of microbes during lyophilization and subsequent storage.

During lyophilization, the influence of lyophilization parameters, freeze-drying matrix and different storage conditions are factors that may impact short- and long-term microbe viability. Lyophilization parameters may relate to the materials and conditions used during the lyophilization. Parameters can include but are not limited to cryoprotectants, media, temperature, pressure, time, the sample volume, and the sample moisture content. Lyophilization media may comprise a cryoprotectant and/or a matrix agent. The matrix agent can direct the entire sample in maintaining its shape during and after the lyophilization. Matrix agents may be selected from the group consisting of skim milk, mannitol, serum, and bovine serum albumin (BSA). The storage conditions may comprise the stable duration, temperature, and atmospheric oxygen conditions for the lyophilized microbe.

One or more isolated and purified anaerobes may be lyophilized with a cryoprotectant. Various cryoprotectant combinations may be used for viability increase of bacteria after freeze drying, texture improvement of the lyophilized cake for easy grinding, and lone term stability improvement of the freeze dried bacteria at different temperature conditions. Cryoprotectant may comprise skim milk powder, whey protein, water, vegetable media, dextran, glutamic acid, histidine, mannitol, trehalose, glycerol, maltodextrin, inulin, betaine, adonitol, sucrose, glucose, lactose and polymers, and any combination thereof. Such combinations can result in viable cells immediately after freeze-drying. Cryoprotectant combinations can comprise sodium glutamate or sorbitol and dextrane, combined with cryoprotectants such as trehalose and sucrose.

The cryoprotectant to strain ratio may be a residual cryoprotectant in Which all of the cryoprotectant supernatant can be removed.

The croprotectant elements may comprise formamide, dimethyl sulfoxide, ethylene glycol, propylene glycol, glycerol, colloids, sucrose, trehalose, inulin, glycerol, trehalose, skim milk, and 2-methyl-2,4-pentanediol, water, and growth media. The cryoprotectant may be at least about 1%, 2%, 3%, 4%, 5%, 6%, 7%, 8%, 9%, 10%, 20%, 30%, 40%, 50%, 60%, 70%, 80%, 90%, or 100% by volume. The cryoprotectant may be at least about 1×, 2×, 3×, 4×, 5×, 6×, 7×, 8×, 9×, 10×, 15×, 20×, 25×, 30×, 35×, or 40× by weight. The cryoprotectant may be at least about 1×. The cryoprotectant may be at most about 1×, 2×, 3×, 4×, 5×, 6×, 7×, 8×, 9×, 10×, 15×, 20×, 25×, 30×, 35×, or 40× by weight. The cryoprotectant may be at most about 1×.

After microbial culture growth, lyophilization buffer may be added to the plate and the cells may be suspended using sterile glass rod. Cryoprotectants may also be added. The culture suspension may be frozen for at least about 20 minutes, 30 minutes, 40 minutes, 50 minutes, 1 hour, 2 hours, 3 hours, 4 hours, 5 hours, 10 hours, 15 hours, 20 hours, or 25 hours. The temperature should be at most about −90° C., −80° C., −70° C., −60° C., −50° C., −40° C., −30° C., −20° C., or −10° C. The microbe may also be flash frozen. Flash freezing can occur in a dry ice and ethanol bath. The freezing method may also use liquid nitrogen. During freezing, the lyophilizer may be turned on and the appropriate temperature and vacuum conditions may be allowed to stabilize. To ensure freezing, the sample may sit at a temperature of at least about −60° C., −50° C., −40° C., −30° C. for about at least 20 minutes, 30 minutes, 40 minutes, 50 minutes, 1 hour, 2 hours, or 3 hours. The lyophilizer may be able to reach a pressure of at most about 75 millitorr (mtorr), 100 mtorr, 125 mtorr, 150 mtorr, 175 mtorr, 200 mtorr, 250 mtorr, 300 mtorr, 350 mtorr, 400 mtorr, 450 mtorr, or 500 mtorr. The duration to reach pressure may be at most about 10 minutes, 20 minutes, 30 minutes, 40 minutes, or 50 minutes. Then, the temperature of the drying shelf may be raised to at most −90° C., −85° C., −80° C., −75° C., −70° C., −65° C., −60° C., −55° C., −50° C., −45° C., −40° C., −35° C., −30° C., −25° C., −20° C., −15° C., or −10° C.

Freeze drying may occur using a shelf or a manifold. The frozen culture may be carefully and aseptically placed in the freeze drying chamber. The sample may be at least about 0.1 milliliter (mL), 0.2 mL, 0.3 mL, 0.4 mL, 0.5 mL, 0.6 mL, 0.7 mL, 0.8 ml, 0.9 mL, or 1 mL. Vacuum may be applied to the chamber. The culture can completely lyophilize out after at least about 1 hour, 2 hours, 3 hours, 4 hours, 5 hours, 10 hours, 15 hours, 20 hours, 25, or 48 hours. The samples may then be removed from the freezer drier chamber and stored at a temperature below about 30° C., 10° C., 0° C., −20° C., −30° C., or −80° C. The sample may also contain at most about 1%, 2%, 3%, 4%, 5%, 6%, 7%, 8%, 9%, 10%, 15%, 20%, 25%, 30%, 35%, 40%, 45%, or 50% of moisture. The sample may also contain at least about 1%, 2%, 3%, 4%, 5%, 6%, 7%, 8%, 9%, 10%, 15%, 20%, 25%, 30%, 35%, 40%, 45%, or 50% of moisture. During the secondary drying phase, the moisture level can be reduced by applying heat onto the sample for at most about 30 minutes, 40 minutes, 50 minutes, 1 hour, 1.5 hours, 2 hours, or 3 hours.

The lyophilized bacteria may be oxygen stable. The lyophilized strains may be stable in the atmosphere containing at least about 10%, 11%, 12%, 13%, 14%, 15%, 16%, 17%, 18%, 19%, 20%, 21%, 22%, 23%, 24%, or 25% oxygen. The stability and viability of the lyophilized cultures may be monitored for at least about 7 days, 14 days, 30 days, 60 days, 90 days, 120 days, 150 days, 180 days, 365 days, or 730 days. The lyophilized culture may be stable at a temperature of at least about 0° C., 5° C., 10° C., 15° C., 20° C., 25° C., or 30° C. The method may yield at least about 0.1%, 0.2%, 0.3%, 0.4%, 0.5%, 0.6%, 0.7%, 0.8%, 0.9%, 1%, 1.2%, 1.4%, 1.6%, 1.8%, 2%, 2.2%, 2.4%, 2.6%, 2.8%, 3%, 3.2%, 3.4%, 3.6%, 3.8%, 4%, 4.2%, 4.4%, 4.6%, 4.8%, 5%, 10%, or 100% viable cells. One or more oxygen stable microbes can be viable in between 0 parts per million (ppm) of oxygen and 100 ppm of oxygen. Oxygen stable microbes may be viable in at most 0.1 ppm, 0.2 ppm, 0.3 ppm, 0.4 ppm, 0.5 ppm, 0.6 ppm, 0.7 ppm, 0.8 ppm, 0.9 ppm, 1 ppm, 1.2 ppm, 1.4 ppm, 1.6 ppm, 1.8 ppm, 2 ppm, 2.2 ppm, 2.4 ppm, 2.6 ppm, 2.8 ppm, 3 ppm, 3.2 ppm, 3.4 ppm, 3.6 ppm, 3.8 ppm, 4 ppm, 4.2 ppm, 4.4 ppm, 4.6 ppm, 4.8 ppm, 5 ppm, 10 ppm, or 100 ppm of oxygen. Oxygen stable microbes may be viable in 0%, 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, or 2% of dissolved oxygen (DO).

In some embodiments, the microbes are manufactured in a dry form, for example, by spray-drying or lyophilization. In some embodiments, the formulation is prepared as a liquid capsule to maintain the liquid form of the microbes.

Lyophilized bacteria may be viable for at least about 1 day, 2, days, 3 days, 4 days, 5 days, 6 days, 7 days, 8 days, 9, days, 10 days, 11 days, 12 days, 13 days, 14 days, 15 days, 16 days, 17 days, 19 days, 20 days, 21 days, 22 days, 23 days, 24 days, 25 days, 26 days, 27 days, 28 days, 29 days, 30 days, 1 month, 2 months, 3 months, 4 months, 5 months, 6 months, 7 months, 8 months, 9 months, 10 months, 11 months, 12 months, 1 year, 2 years, 3 years, 4 years, or 5 years in atmospheric oxygen.

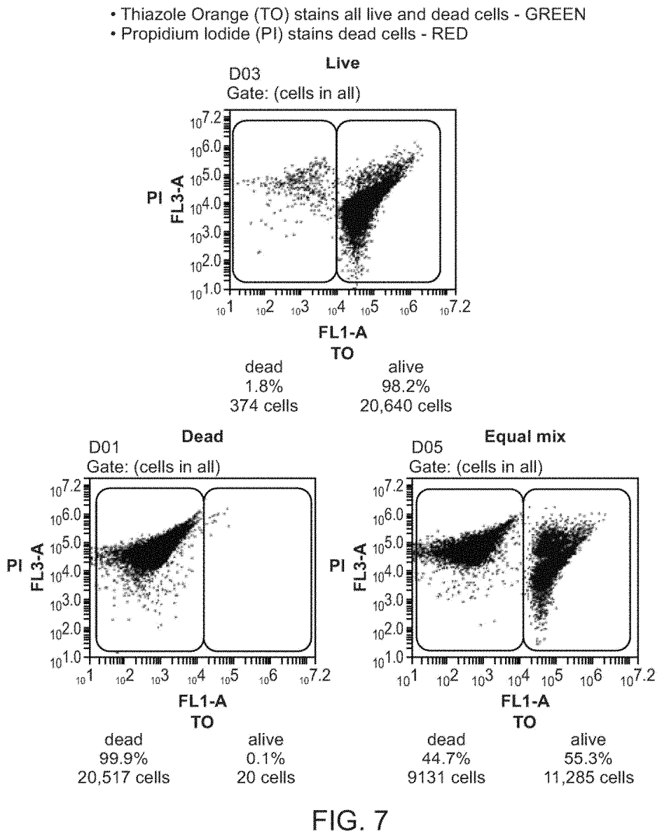

Bacterial detection and live/dead data may be determined after lyophilization by flow cytometry. Flow cytometry may be used to determine the viability, metabolic state, and antigenic markers of bacteria. Specifically, this method can quantify the viable microbes in a sample. For example, live cells may be characterized by undamaged membranes and are impermeable to certain dyes such as propidium iodide (PI). PI may be able to permeate the broken membranes of dead cells. Contrarily, other dyes such as thiazole orange (TO) may be permanent and can enter all cells. The cells may have intact membranes and may be alive or dead. The combination of these two dyes may be used to characterize microbes post-lyophilization in the live/dead assay. FIG. 7 shows a representative live and dead microbial count that can be used to create a standard curve for other measurements. The live/dead data may also be correlated with optical density. For example, a plate reader can measure the optical density and correlate the measurements with CFU. In FIG. 7, the right panel represents thiazole orange stains on all live and dead cells and the left panel indicates propidium iodide that stains the dead cells.

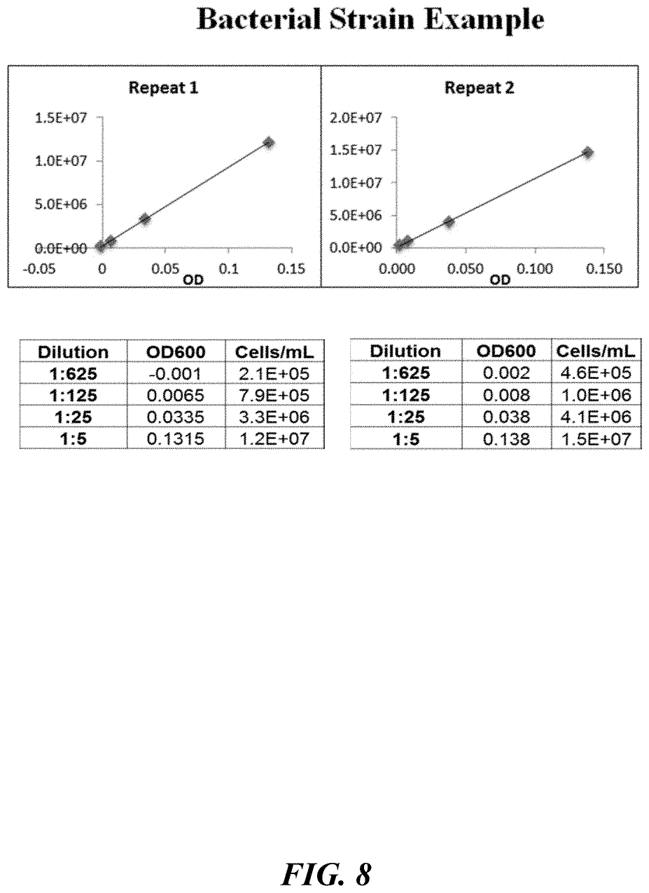

FIG. 8 shows reproducible measurements from a series of dilutions in determining the linear relationship between OD and CFU for B. longum. OD is a fast optical method that simply reports back on the “transparency” of the liquid. CFU is a more difficult measure to obtain since the determination of live versus dead cells needs to be made. The previous standard method for CFU measure was growing the bacteria on a plate and then counting the # of colonies that were observed later (hence the term Colony Forming Unit). A more recent method (the one used in this figure), involves staining the bacterial culture with cell permeable and impermeable dyes to enable differentiation between dead and live cells. Then a fluorescence count is made using a flow cytometer. If the OD method can be normalized using the more involved CFU method then it can be used as a proxy to monitor growth.

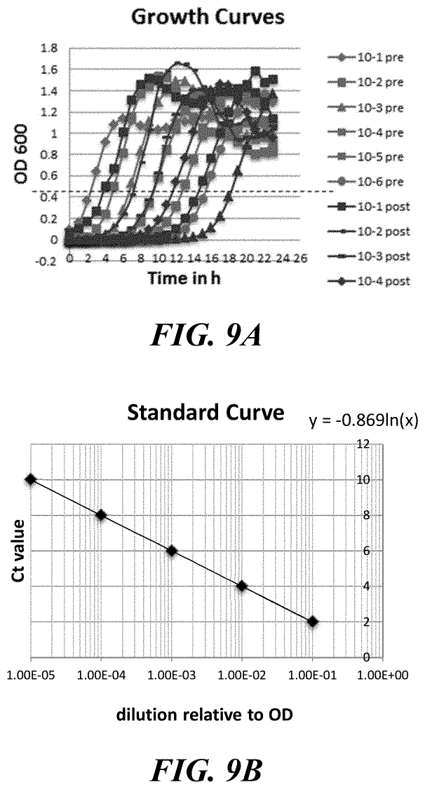

In FIG. 9A, viable bacterial cell counts of B. longum in a 96 well plate are compared before and after lyophilization. Delays observed in the growth curves are directly related to initial starting concentration, so the shift to the right post lyophilization indicates that losses to viability occurred in the process. By using a concentration standard, this delta can be converted into a concentration drop (e.g. 10% viable post lyophilization). Furthermore in FIG. 9B, a standard may be developed in plotting cycle threshold value against the microbial dilutions. This shows quantitative polymerase chain reaction (qPCR) data using specific primers and indicating that we indeed observe the concentration range we expect.

The composition for administration to a subject in need thereof may comprise Roseburia inulinivorans, Methanobrevibacter smithii, Bifidobacterium infantis, chitin, dextrose, ribose, tween, glycerol, and P-glycan.

The composition for administration to a subject in need thereof may comprise Coprococcus, Ruminococcus flavefaciens, Bifidobacterium infantis, biotin, sorbitol, sucrose, ascorbic acid, adonitol, and resistant starch.

The composition for administration to a subject in need thereof may comprise Faecalibacterium prausnitzii, Peptostreptococcus, Bifidobacterium infantis, P-glucan, mannitol, lactose, xanthan gum, glutamic acid, and xylooligosaccharides.

The composition for administration to a subject in need thereof may comprise Clostridium beijerinckii, Clostridium butyricum, Bifidobacterium infantis, inulin, sucrose, trehalose, glycerin, maltodextrin, and hydroxypropyl methylcellulose.

The composition for administration to a subject in need thereof may comprise Akkermansia muciniphila, Bifidobacterium infantis, Clostridium beijerinckii, Clostridium butyricum, Eubacterium hallii, inulin, sucrose, trehalose, glycerin, maltodextrin, and hydroxypropyl methylcellulose.

The composition for administration to a subject in need thereof may comprise Bifidobacterium adolescentis, Akkermansia muciniphila, Eubacterium hallii, and Clostridium indolis, inulin, sucrose, trehalose, glycerin, maltodextrin, and hydroxypropyl methylcellulose.

The composition for administration to a subject in need thereof may comprise Bifidobacterium infantis, Bifidobacterium longum, Clostridium beijerinckii, Clostridium butyricum, and Eubacterium hallii, inulin, sucrose, trehalose, glycerin, maltodextrin, and hydroxypropyl methylcellulose.

The composition for administration to a subject in need thereof may comprise Faecalibacterium prausnitzii, Clostridium beijerinckii, Bifidobacterium bifidum, and Lactobacillus brevis, inulin, sucrose, trehalose, glycerin, maltodextrin, and hydroxypropyl methylcellulose.

The composition for administration to a subject in need thereof may comprise Clostridium beijerinckii, Clostridium butyricum, Bifidobacterium infantis, inulin, sucrose, trehalose, glycerin, maltodextrin, and hydroxypropyl methylcellulose.

The composition for administration to a subject in need thereof may comprise Akkermansia muciniphila, Clostridium beijerinckii, Clostridium butyricum, Eubacterium hallii, Bifidobacterium infantis, inulin, sucrose, trehalose, glycerin, maltodextrin, and hydroxypropyl methylcellulose.

The composition for administration to a subject in need thereof may comprise Clostridium indolis, Bifidobacterium longum, and Akkermansia muciniphila, inulin, sucrose, trehalose, glycerin, maltodextrin, and hydroxypropyl methylcellulose.

The composition for administration to a subject in need thereof may comprise Bifidobacterium bifidum and Lactobacillus brevis, inulin, sucrose, trehalose, glycerin, maltodextrin, and hydroxypropyl methylcellulose.

The composition for administration to a subject in need thereof may comprise Faecalibacterium prausnitzii, Peptostreptococcus, Bifidobacterium infantis, pectin, lactose, mannitol, palm oil, whey protein, and trans-galactooligosaccharide.

The composition for administration to a subject in need thereof may comprise Acidaminococcus intestine, Anaerostipes caccae, Bifidobacterium infantis, tagatose, glucose, sucrose, carrageenan gum, water, and beta-glucans.

The composition for administration to a subject in need thereof may comprise Clostridium orbiscindens, Lactobacillus casei, Bifidobacterium infantis, cellulose, maltose, N-Acetylglucosamine, poly-L-lysine, vegetable media, and locust bean gum.

The composition for administration to a subject in need thereof, wherein composition has the following properties: a) the composition comprises at least 1.0×10⁸ active cells/g, and b) the composition comprises no more than 5.0 mcg/g of arsenic, no more than 3.3 mcg/g of lead, no more than 5.0 mcg/g of mercury, and no more than 1.6 mcg/g of cadmium. The composition can be a powder. The composition can be beige to dark tan in color.

The composition for administration to a subject in need thereof, comprising at least three of the properties selected from the group consisting of: a) the composition comprises about 1.0×10⁸ active cells/g, b) the composition is a powder, c) the composition is beige to dark tan in color, and d) the composition comprises no more than 5.0 mcg/g of arsenic, no more than 3.3 mcg/g of lead, no more than 5.0 mcg/g of mercury, and no more than 1.6 mcg/g of cadmium.

The composition for administration to a subject in need thereof, wherein composition has the following properties: a) the composition comprises about 8.2×10⁹ active cells/g, b) the composition is a powder, c) the composition is tan in color, and d) the composition comprises no more than about 0.02 mcg/g of arsenic, no more than about 0.2 mcg/g of lead, no more than about 0.01 mcg/g of mercury, and no more than about 0.12 mcg/g of cadmium.

The composition for administration to a subject in need thereof, comprising at least three of the properties selected from the group consisting of: a) the composition comprises about 8.2×10⁹ active cells/g, b) the composition is a powder, c) the composition is tan in color, and d) the composition comprises no more than about 0.02 mcg/g of arsenic, no more than about 0.2 mcg/g of lead, no more than about 0.01 mcg/g of mercury, and no more than about 0.12 mcg/g of cadmium.

### Encapsulation Methods

Formulated compositions can be more effective and can be more characterized than food based carrier systems. Examples of formulations for probiotic delivery can comprise capsules, tablets, or beads. The formulation processes can affect the potential of dosage forms to administer the correct amount and number of viable microbes. Additional parameters may be integrated into the compositions to increase the survival rate of the microbe.

The composition comprising one or more isolated and purified microbes, discussed herein may be encapsulated for delivery to a small intestine, a large intestine, an ileum, or a combination thereof, of the subject. The encapsulated mixture may not substantially release the population of isolated and purified microbes prior to a small intestine or a large intestine of the subject.

Encapsulation techniques may be chosen from multiple routes, including shell coating of the formulation in a fluidized bed or pan coater or dispersing the formulation as droplets in an immiscible liquid or air with solidification of the droplets. Dispersion techniques may comprise liquid air dispersion or liquid liquid dispersion. Liquid air dispersion includes atomization and dripping and jet break up. Liquid liquid dispersion includes emulsification and micellization. Atomization may be accomplished by a pressure nozzle, two fluid nozzle, and a spinning disc. Dripping and Jet break up includes simple dripping, electrostatic extrusion, coaxial air and liquid flow, jet cutting, centrifugal nozzle, or vibrating nozzle. Emulsification includes high pressure homogenizers, ultrasound homogenizers, static mixers, rotor and stator devices, microfluidic devices, membrane emulsification, microchannel emulsification, and inkjet printing.

Solvent evaporation and cooling or crosslinking in a hardening bath may solidify air suspended droplets. Emulsification is another method that can involve the emulsification of a suspension or solutions of actives in continuous phase liquid. This can be followed by matrix/shell production by internal gelation, polymerization, layer by layer electrostatic deposition, internal phase separation, and coacervation. The common methods for solid shell and matrix formation in encapsulation processes can be mechanical and thermal, physicochemical, or chemical. Mechanical and thermal methods include cooling, freezing, pan coating, or fluidized-bed coating. Fluidizing bed coating can comprise top spray, bottom spray, tangential spray, or wurster process. Physicochemical methods may include solvent removal, layer by layer deposition, self-assembly, simple and complex coacervation, ionotropic gelation, or internal phase separation. Solvent removal includes evaporation or drying and liquid extraction. Chemical methods can comprise suspension polymerization, interfacial polycondensation, or sol-gel chemistry. Suspension polymerization may comprise one stage (direct) suspension polymerization or two-stage suspension polymerization (droplet swelling) method. Liposome can also be used for encapsulation.

Hydrogels can be used to encapsulate microbes. The microbes may comprise one or more strains. The hydrogels may comprise a hydrophilic active that is captured in a hydrophilic polymer network. Chemical or physical gelation can form the gel networks. Chemical gelation may comprise the polymerization of free-radical processes or condensation. Physical gelation can make use of heating with heat setting gels, cooling with cold setting gels, or addition of multivalent counter ions via ionotropic gelation. Contrarily, coacervation may comprise first an electrostatic phase separation in an emulsion or suspension of the active ingredient into a three phase system containing a polymer rich liquid phase, polymer lean liquid phase, and a liquid or solid phase with the active ingredient. Second, coacervation can comprise deposition of the coaverate phase onto the dispersed droplets or particles followed by a hardening of the coat.

In the solvent evaporation method, an organic solvent can dissolve a high melting point oil and the mixture is emulsified at room temperature with an aqueous phase. Next, the solid particles may be produced by organic solvent evaporation. As a result, the solid lipid particles are smaller than the initial oil droplets. On the other hand, during temperature-controlled emulsification, solid lipid microparticles can generally be the same size as the initial oil droplets. Hydrophilic samples can be encapsulated by forming a water in oil in water emulsion (W/O/W) prior to solvent evaporation or cooling.

### Growth of Microbes as Biofilms

In some cases, biofilm formation may be used as a technique to improve the viability and shelf life of microbes. Microbes may be allowed to generate extracellular matrices that can comprise scaffolds of proteins, sugars, lipids and in some cases extracellular DNA in the form of biofilms. In some cases, biofilm formation may be beneficial for the growth of the microbes. In some cases, the benefits of biofilm formation may include an increase in viability of the microbe in culture.

In some embodiments, the microbes grown as biofilms may be stored after a drying procedure. Non-limiting examples of drying procedures are: lyophilization, freeze-drying, spray drying, etc.

In some embodiments, the microbes grown as biofilms may be lyophilized to increase storage time and viability.

In some embodiments, the microbes grown in biofilms may be coated with cryoprotectants to improve the viability of microbes. Alternatively, microbes in culture may comprise cryoprotectants as part of the culture media.

In some embodiments, the lyophilized microbes may be encapsulated. In some cases, the encapsulation procedure may be microencapsulation. Microencapsulation, in some cases, may be performed to increase the storage time or shelf life of the microbial product at room temperature.

### Methods for Treating a Subject

The disclosure provides methods and compositions for treating a health condition, for example, a microbiome-associated health condition. Treatment can be achieved by, for example, administering a therapeutically-effective amount of a microbial-based composition at a suitable body site that shows a correlated link to disease onset. A composition can be delivered to the gut of a subject. A composition can be administered for release in the gut of a subject.

The disclosure provides methods for the restoration of a microbial habitat of a subject to a healthy state. The method can comprise microbiome correction and/or adjustment including for example, replenishing native microbes, removing pathogenic microbes, administering prebiotics, and growth factors necessary for microbiome survival. The method can comprise administering antimicrobial agents such as antibiotics.

### Microbiome-Associated Disorders

Non-limiting examples of heath conditions that can be associated with the microbiome are presented. These health conditions can include, for example, Type 2 Diabetes Mellitus (T2DM), preterm labor, chronic fatigue syndrome, skin conditions such as acne, allergies, autism, asthma, depression, hypertension, irritable bowel syndrome, metabolic syndrome, obesity, lactose intolerance, oral thrush, ulcerative colitis, drug metabolism, vaginosis, atopic dermatitis, psoriasis, Type I Diabetes Mellitus (T1DM), diabetes, Multiple Sclerosis, neurological disorders such as Parkinson's disease, Clostridium Difficile infection, Inflammatory Bowel Disease, Crohn's Disease, heart disease, diabetic foot ulcers, bacteremia, infantile colic, cancer, cystic fibrosis, multiple sclerosis, urinary tract infection, radiation enteropathy, drug metabolism, dental cavities, halitosis, metabolic disorder, gastrointestinal disorder, insulin insensitivity, metabolic syndrome, insulin defficiency, insulin resistance, glucose intolerance, Non-Alcoholic Fatty Acid Liver Disease (NAFLD), Nonalcoholic steatohepatitis (NASH), Cardiovascular Disease, Hypertension, disorder associated with Cholesterol, disorder associated with Triglycerides, obesity, overweight condition, inflammation, infant formula feeding, appendicitis, atopic disease, ageing, fasting, pregnancy, obesity during pregnancy, dextran sodium sulfate-induced colitis, diarrhea, allergic diarrhea, and atherosclerosis.

In some embodiments, the disorder is associated with and/or caused by an altered microbiome of the subject. In some embodiments, a disorder is associated with and/or caused by gut dysbiosis. In some embodiments, the disorder is associated with and/or caused by an altered production of one or more short chain fatty acids (SCFA) in the subject. In some embodiments, the short chain fatty acid is butyrate. In some embodiments, the short chain fatty acid is propionate (e.g., indole 3-propionate). In some embodiments, the short chain fatty acid is acetate. In some embodiments, the disorder is caused by reduced butyrate production. For example, a patient can have reduced short-chain fatty acid producing (e.g. butyrate-producing) microbes. Altered SCFA production can be caused by, for example, an altered SCFA pathway (e.g., altered butyrate pathway), altered SCFA-producing microbes, or an increase or decrease in substrate or cofactors needed for the SCFA pathway or SCFA-producing microbes. Altered butyrate production can affect one or more downstream signaling pathways in a subject, which can lead to a disorder. Methods and compositions, for example, comprising probiotics to increase butyrate production can be used for treating a disorder.

A subject can have a microbiome profile that is a signature or characteristic of a disorder (e.g., a microbiome signature of a disorder). For example, a patient with a metabolic disorder such as IBD or Crohn's disease can have a reduced population of microbes such as bacteriodes, eubacterium, faecalibacterium and ruminococcus, and/or an increased population of actinomyces and Bifidobacterium. The patient can have reduced butyric acid concentration (e.g., in feces) compared with healthy controls. The microbiota signature of a disorder can be used as a diagnostic for determining a disorder. Imbalance in intestinal microflora constitution can be involved in the pathogenesis of inflammatory bowel disease.

A disorder or condition treated by a composition of the disclosure can include skin or dermatological disorders, metabolic disorders, neurological disorders, cancer, cardiovascular disorders, immune function disorders, inflammatory disorder, pulmonary disorder, metastasis, a chemotherapy or radiotherapy-induced condition, age-related disorder, a premature aging disorder, and a sleep disorders.

Alterations in gut microbiota can be implicated in the pathophysiology of a disorder, for example, skin or dermatological disorders, metabolic disorders, neurological disorders, cancer, cardiovascular disorders, immune function disorders, inflammatory disorder, pulmonary disorder, metastasis, a chemotherapy or radiotherapy-induced condition, age-related disorder, a premature aging disorder, and a sleep disorders.

A subject with a metabolic disorder or metabolic syndrome can suffer from comorbidities including, for example, skin or dermatological disorders, metabolic disorders, neurological disorders, cancer, cardiovascular disorders, immune function disorders, inflammatory disorder, pulmonary disorder, metastasis, a chemotherapy or radiotherapy-induced condition, age-related disorder, a premature aging disorder, and a sleep disorders

### Metabolic Disorders

In some embodiments, the disorder can be a metabolic disorder. Non-limiting examples of metabolic disorders include diabetes, Type I diabetes mellitus, Type II diabetes mellitus, gestational diabetes, juvenile diabetes, metabolic syndrome, inflammatory bowel disease (IBD), irritable bowel syndrome, obesity, overweight condition, ischemia-reperfusion injury such as hepatic ischemia-reperfusion injury, fatty liver disease, non-alcoholic fatty liver disease (NAFLD), non-alcoholic steatohepatitis (NASH), NAFLD in a non-obese subject (e.g., NAFLD not caused by or related to obesity or excess weight problems), NASH in a non-obese subject (e.g., NASH not caused or related to obesity or excess weigh problems), Crohn's disease, colitis, ulcerative colitis, Pseudomembranous colitis, renal dysfunction, nephrological pathology, glomerular disease, drug metabolism, lactose intolerance, insulin insensitivity, insulin deficiency, insulin resistance, glucose intolerance, diarrhea, allergic diarrhea, dextran sodium sulfate-induced colitis.

Patients with metabolic disorders can have reduced butyrate producers. A subject with a metabolic condition (e.g., Crohn's Disease; inflammatory bowel disease) can show a decrease in Bacteroides, Eubacterium, Faecalibacterium and Ruminococcus; and an increase in Actinomyces and Bifidobacterium; a decrease in butyrate production pathway; a decrease in butyrate producing strains; a decrease in butyric acid concentration (e.g., in feces); and imbalance in intestinal microflora constitution.

In some embodiments, the disorder can be Type I diabetes mellitus (T1DM). Patients with T1DM can have reduced bacterial diversity and reduced butyrate producing microbes. Increasing butyrate production, for example by administering a composition comprising A. muciniphila, can be used for T1DM treatment.

In some embodiments, the disorder can be inflammatory bowel disease (IBD). Patients with IBD can have reduced butyrate production (e.g., due to reduced butyrate-producing microbes). Increasing butyrate production can result in decreased IBD. Butyrate can ameliorate colonic inflammation associated with IBD.

In some embodiments, the disorder can be Crohn's disease. Butyrate can, for example, decrease cytokine (e.g., Tumor Necrosis Factor; proinflammatory cytokine mPRA) production; abolish lipopolysaccharide induced expression of cytokines; and abolish transmigration of NFkappaB (NF-kB) to the nucleus in blood cells. Butyrate can decrease proinflammatory cytokine expression, for example, via inhibition of NF-kB activation and IkappaBalpha (IdBa) degradation. Butyrate can inhibit inflammatory responses (e.g., in Crohn's disease) through NF kappa B inhibition.

In some embodiments, the disorder can be non-alcoholic fatty liver disease (NAFLD). In some embodiments, the disorder can be non-alcoholic steatohepatitis (NASH). Subjects with NAFLD can have reduced butyrate production and/or butyrate-producing microbes. Administration of butyrate-producing microbes (e.g. C. butyricum) can, for example, reduce NAFLD progression, reduce hepatic lipid deposition, improve triglyceride content, improve insulin resistance, improve serum endotoxin levels, and improve hepatic inflammatory indexes. Altered gut microbiome can independently cause obesity, which can be one of the most important risk factor for NAFLD. This capability can be attributed to short-chain fatty acids (SCFAs), which are gut microbial fermentation products. SCFAs can account for a large portion of caloric intake of the host. SCFAs can enhance intestinal absorption by activating GLP-2 signaling. Elevated SCFAs can be an adaptive measure to suppress colitis, which could be a higher priority than imbalanced calorie intake. The microbiome of non-alcoholic steatohepatitis (NASH) patients can feature an elevated capacity for alcohol production. The pathomechanisms for alcoholic steatohepatitis can apply to NASH. NAFLD and NASH can be associated with elevated Gram-negative microbiome and endotoxemia. NASH patients can exhibit normal serum endotoxin indicating that endotoxemia may not be required for the pathogenesis of NASH. Microbial compositions of the disclosure can benefit NAFLD and NASH patients.

In some embodiments, the disorder can be total hepatic ischemia reperfusion injury. Butyrate preconditioning can improve hepatic function and histology following ischemia-reperfusion injury. Inflammatory factors levels, macrophages activation, TLR4 expression and neutrophil infiltration can be attenduated by butyrate.

In some embodiments, the disorder can be gestational diabetes.

### Neurological and Behavioral Conditions

In some embodiments, the disorder can be a neurological condition. Neurological conditions include, but are not limited to, neural activity disorders, anxiety, depression, food addiction, chronic fatigue syndrome, autism, autistic spectrum disorder, Asperger syndrome, Pervasive Developmental Disorder, Parkinson's disease, Alzheimer's disease, dementia, amyotrophic lateral sclerosis (ALS), bulbar palsy, pseudobulbar palsy, primary lateral sclerosis, motor neuron dysfunction (MND), mild cognitive impairment (MCI), Huntington's disease, ocular diseases, age-related macular degeneration, glaucoma, vision loss, presbyopia, cataracts, progressive muscular atrophy, lower motor neuron disease, spinal muscular atrophy (SMA), Werdnig-Hoffman Disease (SMA1), SMA2, Kugelberg-Welander Disease (SM3), Kennedy's disease, post-polio syndrome, and hereditary spastic paraplegia. Compositions of the disclosure can be used, for example, for stabilizing mood, improving mood, modulating excessive emotional distress, reducing anxiety, reducing stress, and combinations thereof. In some embodiments, the disorder is a behavioral condition.

Gut microbes can play a role in nervous system and host behavior. Increasing SCFA production (e.g., by increasing butyrate producers) can, for example, improve brain development, motor activity, reduce anxiety, improve depression, increased immunoregulatory Treg cells, and improved psychological states.

Methods and compositions of the disclosure can regulate, for example, hypothalamus-ptuitary-adrenal axis (HPA), immune systems, enteric nervous system, autonomic nervous system, central nervous system, production of neuroactive substances, production of short chain fatty acids (SCFAs), production of antibiotic active substances, and altered intestinal function (e.g, sensory-motor function, barrier function).

Methods and compositions of the disclosure can regulate behavior by, for example, regulation of cortisol, serotonin, dopamine, and/or GABA. Methods and compositions of the disclosure can be used to regulate appetite by, for example, regulation of insulin, leptin, ghrelin, and/or GLP-1.

Methods and compositions of the disclosure can regulate intestinal immune system by, for example, regulation of mast cell activation and/or inflammatory cytokine production.

Butyrate can activate intestinal gluconeogenesis in insulin-sensitive and insulin-insensitive states, which can promote glucose and energy homeostasis. Microbial compositions can alter activity in brain regions that control central processing of emotion and sensation.

Methods and compositions of the disclosure can modulate (e.g., reduce) appetite in a subject. Methods and compositions can modulate (e.g., improve) behavior of a subject. Methods and compositions of the disclosure can modulate (e.g., promote) satiety in a subject.

Butyrate production by gut microbiome can decrease appetite, for example, via gut-brain connection. Obese subjects can have increased scores on food addition and food cravings scales when compared to lean subjects. Alterations in gut microbiota can be implicated in the pathophysiology of several brain disorders including anxiety, depression, and appetite. When fiber is ingested, gut microbes can metabolize the fiber into short chain fatty acids, including butyrate. Butyrate can bind to receptors, for example, G-protein coupled receptors. For example, butyrate can bind to G-protein coupled receptor GPR41 and trigger peptide tyrosine-tyrosine (PYY) and glucagon-like peptide 1 (GLP-1). PYY and GLP-1 can bind to receptors in the enteric nervous system, resulting in signaling to the brain via the vagus nerve that can result in reducing appetite.

Methods and compositions of the disclosure can alter levels of neurotransmitters substance (e.g., serotonin, dopamine, GABA), neuroactive metabolite (e.g., branched chain and aromatic amino acids, p cresol, N acetyl putrescine, o cresol, phenol sulfate, kinurate, caproate, histamine, agmatine), and inflammatory agents (e.g., lipopolysaccharide, IL-1, IL-6, IL-8, TNF-alpha, CRP) in a subject.

A microbial composition of the disclosure can produce or regulate production of propionate, for example, indole 3-propionate. Indole-3-propionate can function as an antioxidant. Indole-3-propionate can be associated with neurological disorders, e.g., Alzheimer's disease. Indole-3-propionate can protect neurons and neuroblastoma cells from beta-amyloid protein toxicity. Indole-3-propionate can be produced from, for example, dietary tryptophan by microbes such as Clostridium sporogenes in the gastrointestinal tract. A microbial composition of the disclosure comprising an isolated and purified population of a microbe comprising at least about 85% (e.g., 90%, 95%, 98%, 99% or 100%) sequence identity to a rRNA (e.g., 16S or 23S) sequence of Clostridium sporogenes can be used to treat a neurological disorder (e.g., Alzheimer's disease).

### Immune System Conditions

In some embodiments, the disorder can be an immune system disorder. In some embodiments, the disorder can be an inflammatory condition. In some embodiments, the disorder can be inflammation.

Non-limiting examples of immune system related disorders include allergies, inflammation, inflammatory disorder, anaphylactic shock, autoimmune diseases, rheumatoid arthritis, systemic lupus erythematosus (SLE), scleroderma, diabetes, Autoimmune enteropathy, Coeliac disease, Crohn's disease, Microscopic colitis, ulcerative colitis, osteoarthritis, osteoporosis, oral mucositis, inflammatory bowel disease, kyphosis, herniated intervertebral disc, ulcerative asthma, renal fibrosis, liver fibrosis, pancreatic fibrosis, cardiac fibrosis, skin wound healing, and oral submucous fibrosis.

In some embodiments, the disclosure provides methods for treating or reducing the likelihood of conditions resulting from a host immune response to an organ transplant in a subject in need thereof. Non-limiting examples of an organ transplant include a kidney organ transplant, a bone marrow transplant, a liver transplant, a lung transplant, and a heart transplant. In some embodiments, the disclosure provides methods for treating graft-vs-host disease in a subject in need thereof.

Microbial metabolites can play a role in development of the immune system. Gut microbiome can play a role in the development of allergies. Microbes can mediate immunomodulation. Based on the immunomodulating capacities of bacteria, probiotics can be used for treating eczema, for example, Bifidobacterium bifidum, Bifidobacterium animalis subsp. Lactis, and Lactococcus lactis. Lower amounts of metabolites, SCFAs, succinate, phenylalanine, and alanine can be found in faecal samples of subjects (e.g., children) later developing skin disorders (e.g, eczema), whereas the amounts of glucose, galactose, lactate and lactose can be higher compared to the subjects not developing skin disorders. Supplementation of multispecies probiotics can induce higher levels of lactate and SCFAs, and lower levels of lactose and succinate.

Administration of compositions comprising SCFA or SCFA-producing microbes can increase immunoregulatory cells.

### Skin Disorders

In some embodiments, the disorder can be a dermatological disorder. Dermatological conditions include, but are not limited to, skin health, acne, psoriasis, eczema, rashes, rhytides, pruritis, dysesthesia, papulosquamous disorders, erythroderma, lichen planus, lichenoid dermatosis, atopic dermatitis, eczematous eruptions, eosinophilic dermatosis, reactive neutrophilic dermatosis, pemphigus, pemphigoid, immunobullous dermatosis, fibrohistocytic proliferations of skin, cutaneous lymphomas, and cutaneous lupus.

In some embodiments, the disorder can be atopic dermatitis. In some embodiments, the disorder can be eczema.

Patients with skin disorders (e.g, atopic dermatitis) can have, for example, reduced butyrate producing microbes, lower diversity of the phylum Bacteriodetes, altered diversity of gut microbiome, and altered abundance of C. eutactus.

### Cardiovascular Conditions

In some embodiments, the disorder can be a cardiovascular disorder. Non-limiting examples of cardiovascular conditions, include, but are not limited to angina, arrhythmia, atherosclerosis, cardiomyopathy, congestive heart failure, coronary artery disease (CAD), carotid artery disease, endocarditis, heart attack, coronary thrombosis, myocardial infarction (MI), high blood pressure/hypertension, aortic aneurysm, brain aneurysm, cardiac fibrosis, cardiac diastolic dysfunction, hypercholesterolemia/hyperlipidemia, heasrt disease, mitral valve prolapse, peripheral vascular disease, peripheral artery disease (PAD), cardiac stress resistance, stroke, disorder associated with Cholesterol, disorder associated with Triglycerides.

### Pulmonary Conditions

In some embodiments, the disorder can be a pulmonary condition. Pulmonary conditions include, but are not limited to, idiopathic pulmonary fibrosis (IPF), chronic obstructive pulmonary disease (COPD), asthma, cystic fibrosis, bronchiectasis, and emphysema.

In some embodiments, the subject may have been exposed to environmental pollutants, for example, silica. A subject may have been exposed to an occupational pollutant, for example, dust, smoke, asbestos, or fumes. In some embodiments, the subject has smoked cigarettes.

In some embodiments, the subject can have a connective tissue disease. The connective tissue disease can be, for example, rheumatoid arthritis, systemic lupus erythematosus, scleroderma, sarcoidosis, or Wegener's granulomatosis. In some embodiments, the subject has an infection. In some embodiments, the subject has taken or is taking medication (e.g., amiodarone, bleomycin, busufan, methotrexate, or nitrofurantoin) or has received radiation therapy to the chest.

### Cancer

In some embodiments, the disorder can be cancer. Non-limiting examples of cancers include: colorectal cancer, acute lymphoblastic leukemia, acute myeloid leukemia, adrenocortical carcinoma, AIDS-related cancers, AIDS-related lymphoma, anal cancer, appendix cancer, astrocytomas, neuroblastoma, basal cell carcinoma, bile duct cancer, bladder cancer, bone cancers, brain tumors, such as cerebellar astrocytoma, cerebral astrocytoma/malignant glioma, ependymoma, medulloblastoma, supratentorial primitive neuroectodermal tumors, visual pathway and hypothalamic glioma, breast cancer, bronchial adenomas, Burkitt lymphoma, carcinoma of unknown primary origin, central nervous system lymphoma, cerebellar astrocytoma, cervical cancer, childhood cancers, chronic lymphocytic leukemia, chronic myelogenous leukemia, chronic myeloproliferative disorders, colon cancer, cutaneous T-cell lymphoma, desmoplastic small round cell tumor, endometrial cancer, ependymoma, esophageal cancer, Ewing's sarcoma, germ cell tumors, gallbladder cancer, gastric cancer, gastrointestinal carcinoid tumor, gastrointestinal stromal tumor, gliomas, hairy cell leukemia, head and neck cancer, heart cancer, hepatocellular (liver) cancer, Hodgkin lymphoma, Hypopharyngeal cancer, intraocular melanoma, islet cell carcinoma, Kaposi sarcoma, kidney cancer, laryngeal cancer, lip and oral cavity cancer, liposarcoma, liver cancer, lung cancers, such as non-small cell and small cell lung cancer, lymphomas, leukemias, macroglobulinemia, malignant fibrous histiocytoma of bone/osteosarcoma, medulloblastoma, melanomas, mesothelioma, metastatic squamous neck cancer with occult primary, mouth cancer, multiple endocrine neoplasia syndrome, myelodysplastic syndromes, myeloid leukemia, nasal cavity and paranasal sinus cancer, nasopharyngeal carcinoma, neuroblastoma, non-Hodgkin lymphoma, non-small cell lung cancer, oral cancer, oropharyngeal cancer, osteosarcoma/malignant fibrous histiocytoma of bone, ovarian cancer, ovarian epithelial cancer, ovarian germ cell tumor, pancreatic cancer, pancreatic cancer islet cell, paranasal sinus and nasal cavity cancer, parathyroid cancer, penile cancer, pharyngeal cancer, pheochromocytoma, pineal astrocytoma, pineal germinoma, pituitary adenoma, pleuropulmonary blastoma, plasma cell neoplasia, primary central nervous system lymphoma, prostate cancer, rectal cancer, renal cell carcinoma, renal pelvis and ureter transitional cell cancer, retinoblastoma, rhabdomyosarcoma, salivary gland cancer, sarcomas, skin cancers, skin carcinoma merkel cell, small intestine cancer, soft tissue sarcoma, squamous cell carcinoma, stomach cancer, T-cell lymphoma, throat cancer, thymoma, thymic carcinoma, thyroid cancer, trophoblastic tumor (gestational), cancers of unkown primary site, urethral cancer, uterine sarcoma, vaginal cancer, vulvar cancer, Waldenstrom macroglobulinemia, and Wilms tumor, metastasis.

In some embodiments, the disorder can be colorectal cancer.

Subjects with cancer can have altered butyrate production, for example, due to reduced butyrate-producing microbes. Methods and compositions of the disclosure can be used for tumor treatment and reduction, for example, by delivering butyrate producing microbes to the subject.

Most cell types in the body can utilize glucose as their primary energy source, while normal colonocytes can rely on butyrate for about 60-70% of their energy. Butyrate can undergo beta-oxidation in the mitochondria, which can support energy homeostasis for rapid cell proliferation of the colonic epithelium. In contrast, tumor cells (e.g., colorectal tumor cells) can switch to glucose utilization and aerobic glycolysis. As a result of this metabolic shift, butyrate may not metabolize in the mitochondria of tumor cells to the same extent and can accumulate in the nucleus. In the nucleus, butyrate can function as a histone deacetylase (HDAC) inhibitor to epigenetically regulate gene expression. Patients with colitis can have, for example, up to a 10-fold increase of colorectal cancer.

Methods and compositions of the disclosure can increase levels of butyrate, which can serve as an endogenous HDAC inhibitor. Since bioavailability of butyrate can be primarily restricted to the colon, butyrate may not have adverse effects associated with synthetic HDAC inhibitors such as those used in chemotherapy. Butyrate can target tumor cells, for example, because of the Warburg effect.

Dietary risk of cancer (e.g., colon cancer) can be mediated by dysbiosis of gut microbiota and their metabolites (e.g., SCFAs such as butyrate). Dietary fiber and/or complex carbohydrates can promote saccharolytic fermentation, which can yield anti-inflammatory and antiproliferative SCFAs such as butyrate. Red meat can generate inflammatory and genotoxic metabolites by promoting proteolytic fermentation, hydrogen sulfide production from the sulfur-rich amino acid content of red meat, and expose colonic mucosa to carcinogenic constituents.

Dietary fiber intake can promote a healthy gut microbiome, which in turn can enhance SCFA (e.g., butyrate, acetate, propionate) production. Enhanced SCFA production can result in, for example, reduced food intake, increased energy levels, better colon health, promote healthy gut intestinal barrier, reduce colon content transit time and exposure to carcinogens, cancer cell cycle arrest and apoptosis, inhibition of cancer cell migration and invasion, inhibition of early colon lesion, inhibition of adenoma formation, inhibition of colon adenoma, inhibition of tumor progression, and inhibition of colon carcinoma.

### Vaginal Conditions

In some embodiments, the disorder can be a vaginal condition. Non-limiting examples of vaginal conditions include: vaginosis, bacterial vaginosis, Viral vaginosis, Vulvovaginitis, Yeast infection, preterm labor, Fertility-associated conditions (e.g., low fertility), Trichomonas, Vulvodynia douche follow-up treatment (e.g., for anything people are douching for), vulvar vestibulitis, Vulvodynia, vaginal douching. Compositions of the disclosure can be used after douching (e.g., after douching in subject with vulvodynia).

### Dental Conditions

In some embodiments, the disorder can be a dental condition. Non-limiting examples of dental conditions include: vaginosis, bacterial vaginosis, Viral vaginosis, Vulvovaginitis, Yeast infection, preterm labor, Fertility-associated conditions (e.g., low fertility), Trichomonas, Vulvodynia douche follow-up treatment (e.g., for anything people are douching for), vulvar vestibulitis, Vulvodynia, vaginal douching. Compositions of the disclosure can be used after douching (e.g., after douching in subject with vulvodynia).

### Pregnancy Related Conditions

In some embodiments, the disorder can be a pregnancy related condition. Non-limiting examples of pregnancy related conditions include: preterm delivery, preterm labor, obesity during pregnancy, gestational diabetes. Compositions of the disclosure can be administered to a pregnant woman carrying an infant to be born via C-section and/or to an infant born via C-section. Compositions of the disclosure can be administered to infants, pregnant women, or both for decreasing occurrence of intestinal pathogens or any of the disorders described herein in those infants.

### Formulations

Provided herein are compositions that may be administered as therapeutics (e.g., pharmaceutical compositions) and/or cosmetics. The composition can be administered as a medical food. A medical food may be delivered and administered enterally in the presence of a physician or doctor. A medical food can be administered for specific dietary control of a disease condition for which distinctive nutritional requirements, created upon familiar scientific principles may be determined by medical assessment.

One or more microorganisms described herein can be used to create a formulation comprising an effective amount of the composition for treating a subject. Some non-limiting examples can include topical, capsule, pill, enema, liquid, injection, and the like. In some embodiments, the one or more strains disclosed herein may be included in a food or beverage product, cosmetic, or nutritional supplement.

The formulation can include one or more additional active ingredients. Active ingredients can be selected from the group consisting of: antibiotics, prebiotics, probiotics, glycans (e.g., as decoys that would limit specific bacterial/viral binding to the intestinal wall), bacteriophages, microorganisms and the like.

A composition can include microbes that are, for example, whole microbes (e.g., whole bacteria). The composition can include, for example, viable (e.g., live), dormant, inactivated, or dead microbes (e.g., bacteria). In some embodiments, the composition can include a live microbial strain. In some embodiments, the composition can include a dead microbial strain. In some embodiments, the composition can include a live and a dead microbial strain. The microbial strain can be, for example, any microbial strain disclosed herein.

A composition can include microbial components, for example, cellular components, cellular fractions, proteins (e.g., membrane proteins, soluble proteins), degradation products, metabolites, nucleic acids, secreted molecules and compounds resulting from the metabolism of a microbe. A microbial component (e.g., membrane proteins) can be beneficial for a microbial formulation, for example, to increase stability and viability of microbes in the composition. Microbial components can be obtained, for example, by recovering the supernatant of a microbial culture. Microbial components can be obtained, for example, by extracting cell components or cell fractions, metabolites or secreted compounds from a microbial culture. A microbial component can be a component in the isolated form or a mixture of one or more components. A microbial component can be a purified microbial component. A mixture of purified microbial components can be used.

In some embodiments, the formulation comprises a prebiotic. The prebiotic may be selected from the group comprising trans-galactooligosaccharide, inulin, larch arabinogalactin (LAG), resistant starch, pectin, beta-glucans, xylooligosaccharides, locust bean hum, P-glycan, and methylcellulose. The prebiotic may comprise inulin. The inulin may be present in an amount of at least about 30 milligram/milliliter (mg/mL), 35 mg/mL, 40 mg/mL, 45 mg/mL, 50 mg/mL, 55 mg/mL, 60 mg/mL, 65 mg/mL, or 70 mg/mL in the composition. The inulin can serve as an energy source for the microbial formulation.

A formulation can be administered by a suitable method for delivery to any part of the gastrointestinal tract of a subject including oral cavity, mouth, esophagus, stomach, duodenum, small intestine regions including duodenum, jejunum, ileum, and large intestine regions including cecum, colon, rectum, and anal canal. In some embodiments, the composition is formulated for delivery to the ileum and/or colon regions of the gastrointestinal tract.

In some embodiments, administration of a formulation occurs orally, for example, through a capsule, pill, powder, tablet, gel, or liquid, designed to release the composition in the gastrointestinal tract. In some embodiments, administration of a formulation occurs by injection, for example, for a formulation comprising butyrate, propionate, acetate, and short-chain fatty acids. In some embodiments, the administration of a formulation occurs by application to the skin, for example, cream, liquid, or patch. In some embodiments, administration of a formulation occurs by a suppository and/or by enema. In some embodiments, a combination of administration routes is utilized.

Microbial compositions can be formulated as a dietary supplement. Microbial compositions can be incorporated with vitamin supplements. Microbial compositions can be formulated in a chewable form such as a probiotic gummy. Microbial compositions can be incorporated into a form of food and/or drink. Non-limiting examples of food and drinks where the microbial compositions can be incorporated include, for example, bars, shakes, juices, infant formula, beverages, frozen food products, fermented food products, and cultured dairy products such as yogurt, yogurt drink, cheese, acidophilus drinks, and kefir.

A formulation of the disclosure can be administered as part of a fecal transplant process. A formulation can be administered to a subject by a tube, for example, nasogastric tube, nasojejunal tube, nasoduodenal tube, oral gastric tube, oral jejunal tube, or oral duodenal tube. A formulation can be administered to a subject by colonoscopy, endoscopy, sigmoidoscopy, and/or enema.

In some embodiments, the microbial composition is formulated such that the one or more microbes can replicate once they are delivered to the target habitat (e.g. the gut). In one non-limiting example, the microbial composition is formulated in a pill, such that the pill has a shelf life of at least 1 week, 2 weeks, 3 weeks, 1 month, 2 months, 3 months, 4 months, 5 months, 6 months, 7 months, 8 months, 9 months, 10 months, 11 months, or 12 months, 24 months, and 36 months when stored at 4° C. In one non-limiting example, the microbial composition is encapsulated, such encapsulated product has a shelf life of at least 1 week, 2 weeks, 3 weeks, 1 month, 2 months, 3 months, 4 months, 5 months, 6 months, 7 months, 8 months, 9 months, 10 months, 11 months, or 12 months, 24 months, and 36 months when stored at 4° C. In another non-limiting example, the storage of the microbial composition is formulated so that the microbes can reproduce once they are in the gut. In some embodiments, other components may be added to aid in the shelf life of the microbial composition. In some embodiments, one or more microbes may be formulated in a manner that it is able to survive in a non-natural environment. For example, a microbe that is native to the gut may not survive in an oxygen-rich environment. To overcome this limitation, the microbe may be formulated or encapsulated in a pill, which can reduce or eliminate the exposure to oxygen. Other strategies to enhance the shelf-life of microbes may include other microbes (e.g. if the bacterial consortia comprises a composition whereby one or more strains is helpful for the survival of one or more strains).

A microbial composition can be lyophilized (e.g., freeze-dried) and formulated as a powder, tablet, enteric-coated capsule (e.g. for delivery to ileum/colon), or pill that can be administered to a subject by any suitable route. The composition obtained directly after lyophilization may be a dry powder obtained without further processing (e.g. grinding or crushing). The microbial composition (e.g., powder) can be non-sticky. The microbial composition (e.g., powder) can be sticky. The microbial composition (e.g., powder) can be free-flowing. The microbial composition (e.g., powder) can be, for example, substantially non-sticky, substantially sticky, or substantially free-flowing. The particles in the microbial composition can be cohesive or non-cohesive. In some cases, the particles in the microbial composition can be substantially non-cohesive. The microbial composition (e.g., powder) can have a uniform particle size. The microbial composition (e.g., powder) can have a non-uniform particle size (e.g., particles of different sizes or ranges). The particle size can be, for example, less than 2 mesh size, from about 2 to about 10 mesh size, from about 10 to about 20 mesh size, from about 20 to about 40 mesh size, from about 40 to about 80 mesh size, from 80 to about 120 mesh size, from about 120 to about 200 mesh size, more than 200 mesh size, or a mixture thereof. The microbial composition (e.g., powder) can have a fluid content (e.g., water content) of, for example, about: 0.1 wt %, 0.2 wt %, 0.3 wt %, 0.4 wt %, 0.5 wt %, 0.6 wt %, 0.7 wt %, 0.8 wt %, 0.9 wt %, 1 wt %, 2 wt %, 3 wt %, 4 wt %, 5 wt %, 6 wt %, 7 wt %, 8 wt %, 9 wt %, 10 wt %, 15 wt %, 20 wt %, 25 wt %, 30 wt %, 35 wt %, 40 wt %, 45 wt %, 50 wt %, or more. The microbial composition (e.g., powder) can have a fluid content (e.g., water content) of, for example, at most about: 0.1 wt %, 0.2 wt %, 0.3 wt %, 0.4 wt %, 0.5 wt %, 0.6 wt %, 0.7 wt %, 0.8 wt %, 0.9 wt %, 1 wt %, 2 wt %, 3 wt %, 4 wt %, 5 wt %, 6 wt %, 7 wt %, 8 wt %, 9 wt %, 10 wt %, 15 wt %, 20 wt %, 25 wt %, 30 wt %, 35 wt %, 40 wt %, 45 wt %, 50 wt %, or more. The microbial composition (e.g., powder) can have a fluid content (e.g., water content) of, for example, at least about: 0.1 wt %, 0.2 wt %, 0.3 wt %, 0.4 wt %, 0.5 wt %, 0.6 wt %, 0.7 wt %, 0.8 wt %, 0.9 wt %, 1 wt %, 2 wt %, 3 wt %, 4 wt %, 5 wt %, 6 wt %, 7 wt %, 8 wt %, 9 wt %, 10 wt %, 15 wt %, 20 wt %, 25 wt %, 30 wt %, 35 wt %, 40 wt %, 45 wt %, or 50 wt %. The lyophilized formulation can be mixed with a saline or other solution prior to administration.

A composition can be formulated for oral delivery, for example, as an enteric-coated capsule or pill, for delivery of the contents of the formulation to the ileum and/or colon regions of a subject.

A composition can be formulated for oral administration. In some embodiments, the composition is formulated as an enteric-coated pill or capsule for oral administration. In some embodiments, the composition is formulated for delivery of the microbes to the ileum region of a subject. In some embodiments, the composition is formulated for delivery of the microbes to the colon region (e.g. upper colon) of a subject. In some embodiments, the composition is formulated for delivery of the microbes to the ileum and colon regions of a subject.

An enteric-coating can protect the contents of the oral formulation, for example, pill or capsule, from the acidity of the stomach and provide delivery to the ileum and/or upper colon regions. Non-limiting examples of enteric coatings include pH sensitive polymers (e.g., eudragit FS30D), methyl acrylate-methacrylic acid copolymers, cellulose acetate succinate, hydroxy propyl methyl cellulose phthalate, hydroxy propyl methyl cellulose acetate succinate (e.g., hypromellose acetate succinate), polyvinyl acetate phthalate (PVAP), methyl methacrylate-methacrylic acid copolymers, shellac, cellulose acetate trimellitate, sodium alginate, zein, other polymers, fatty acids, waxes, shellac, plastics, plant fibers, and Capsugel DR. The packaging technology in maintaining the potency may be Bel-Art, Biorx, ColorSafe, CSP Vials, Dynalon, MP Vials, PSA, Pill Pod, Qorpak, Safer Lock, or Wheaton. In some embodiments, the enteric coating is formed by a pH sensitive polymer. In some embodiments, the enteric coating is formed by eudragit FS30D. The enteric coated capsule may comprise at least about 1, 2, 3, 4, 5, 6, 7, 8, 9, or 10 enteric coatings.

The enteric coating can be designed to dissolve at any suitable pH. In some embodiments, the enteric coating is designed to dissolve at a pH greater than about pH 6.5 to about pH 7.0. In some embodiments, the enteric coating is designed to dissolve at a pH greater than about pH 6.5. In some embodiments, the enteric coating is designed to dissolve at a pH greater than about pH 7.0. The enteric coating can be designed to dissolve at a pH greater than about: 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, or 7.5 pH units.

FIG. 4 provides a non-limiting embodiment of stability of individual strains at room temperature (RT) and at 4° C. A microbe can be stable at room temperature and at 4° C. A microbe can be stable for at least at least 5 days, at least 10 days, at least 15 days, or at least 30 days. A microbe can be stable for up to 30 days or up to 15 days.

FIG. 5A provides a non-limiting embodiment of stability of the capsule formulation at room temperature (RT) and at 4° C. FIG. 5B provides additional non-limiting embodiments of two formulations that remain stable over a period of 28 days. The capsule can be stable for at least 5 days, at least 10 days, at least 15 days, at least 25 days, or at least 30 days.

Polymer coatings may include polyvinyl alcohol (PVA), hydroxypropyl methyl cellulose, and hydroxypropyl cellulose, plasticizers, and optional colorants. Film coating for capsules or tables may comprise polyvinyl alcohol (PVA), titanium dioxide, polyethylene glycol, talc, and colorant. The outer protective coat may also include anti-adherens, glidants, and opacifying agents. At least 1, 2, 3, 4, or 5 capsules may be used for targeted delivery. The formulated composition may comprise one or more enteric coatings. The multiple coatings can dissolv in phases that allow for selective delivery. The multiple coatings can also provide controlled release of the composition. The total amount of enteric polymer coating required to achieve colonic release can be reduced if individual multiple enteric polymer coating layers are used. For example, the outermost layer can consist of an enteric polymer that begins to dissolve at least about a pH of 6.0, 6.1, 6.2, 6.3, 6.4, 6.5, 6.6, 6.7, 6.8, 6.9, 7.0, 7.1, 7.2, 7.3, 7.4, or 7.5 in an amount such that this coating layer is completely dissolved within the distal portion of the ileum (small intestine), The inner coating layer(s) may comprise enteric polymers that start to dissolve at least about a pH 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, or 6.5 in an amount in which complete dissolution occurs within the proximal colon. As a result, the outermost coating layer can be used to prevent release of the therapeutic agent as the dosage form transits the gastrointestinal tract to the distal small intestine. The inner coating layer(s) can function to further delay release of the therapeutic agent until the dosage form has reached the proximal colon.

Prior to administration of a formulation, the colon may be cleansed to remove unwanted bacteria, mucous, and old fecal matter in preparation for intake and absorption of the microbial formulation. In some embodiments, the administration of a formulation of the disclosure can be preceded by, for example, colon cleansing methods such as colon irrigation/hydrotherapy, enema, administration of laxatives, dietary supplements, dietary fiber, enzymes, and magnesium.

In some embodiments, the microbes are formulated as a population of microbes with proportionally small amounts of spores. The formulated composition may be substantially free of spores. Spore-containing formulations can be administered by any suitable route described herein. Orally administered spore-containing formulations can survive the low pH environment of the stomach. The amount of spores employed can be, for example, from about 1% w/w to about 99% w/w of the entire formulation. A formulated composition may be substantially free of spores. An exemplary composition may comprise less than about 1%, 3%, 5%, 7%, 9%, 10%, 20%, 30%, 40%, or 50% w/w spores. The mixture of microbes may contain at least about 90% of non-sporulated obligate anaerobes. The proportion of composition comprising spores can be determined by microscopy using a disposable Neubauer chamber. Spores can be easily detectable in random samples in the microscope without further staining.

Formulations provided herein can include the addition of one or more agents to the therapeutics or cosmetics in order to enhance stability and/or survival of the microbial formulation. Non-limiting example of stabilizing agents include genetic elements, glycerin, ascorbic acid, skim milk, lactose, tween, alginate, xanthan gum, carrageenan gum, mannitol, palm oil, and poly-L-lysine (POPL).

In some embodiments, a formulation comprises one or more recombinant microbes or microbes that have been genetically modified. In other embodiments, one or more microbes are not modified or recombinant. In some embodiments, the formulation comprises microbes that can be regulated, for example, a microbe comprising an operon or promoter to control microbial growth. Microbes of the disclosure can be produced, grown, or modified using any suitable methods, including recombinant methods.

In some embodiments, a composition may be formulated for administration before, during, and/or after treatment with an antimicrobial agent such as an antibiotic. For example, the formulation can be administered at least about 1 hour, 2 hours, 5 hours, 12 hours, 1 day, 3 days, 1 week, 2 weeks, 1 month, 6 months, or 1 year before and/or after treatment with an antibiotic. The formulation can be administered at most 1 hour, 2 hours, 5 hours, 12 hours, 1 day, 3 days, 1 week, 2 weeks, 1 month, 6 months, or 1 year before and/or after treatment with an antibiotic.

In some embodiments, the composition may be formulated for administration after treatment with an antibiotic. For example, the formulation can be administered after the entire antibiotic regimen or course is complete.

In some embodiments, a formulation is administered before, during, and/or after food intake by a subject. In some embodiments, the formulation is administered with food intake by the subject. In some embodiments, the formulation is administered with (e.g., simultaneously) with food intake.

In some embodiments, the formulation is administered before food intake by a subject. In some embodiments, the formulation is more effective or potent at treating a microbial condition when administered before food intake. For example, the formulation can be administered about 1 minute, about 2 minutes, about 3 minutes, about 5 minutes, about 10 minutes, about 15 minutes, about 30 minutes, about 45 minutes, about 1 hour, about 2 hours, about 3 hours, about 4 hours, about 5 hours, about 6 hours, about 7 hours, about 8 hours, about 9 hours, about 10 hours, about 12 hours, or about 1 day before food intake by a subject. For example, the formulation can be administered at least about 1 minute, about 2 minutes, about 3 minutes, about 5 minutes, about 10 minutes, about 15 minutes, about 30 minutes, about 45 minutes, about 1 hour, about 2 hours, about 3 hours, about 4 hours, about 5 hours, about 6 hours, about 7 hours, about 8 hours, about 9 hours, about 10 hours, about 12 hours, or about 1 day before food intake by a subject. For example, the formulation can be administered at most about 1 minute, about 2 minutes, about 3 minutes, about 5 minutes, about 10 minutes, about 15 minutes, about 30 minutes, about 45 minutes, about 1 hour, about 2 hours, about 3 hours, about 4 hours, about 5 hours, about 6 hours, about 7 hours, about 8 hours, about 9 hours, about 10 hours, about 12 hours, or about 1 day before food intake by a subject.

In some embodiments, the formulation is administered after food intake by the subject. In some embodiments, the formulation is more effective or potent at treating a microbial condition when administered after food intake. For example, the formulation can be administered at least about 1 minute, 2 minutes, 3 minutes, 5 minutes, 10 minutes, 15 minutes, 30 minutes, 45 minutes, 1 hour, 2 hours, 3 hours, 5 hours, 10 hours, 12 hours, or 1 day after food intake by a subject. For example, the formulation can be administered at most about 1 minute, 2 minutes, 3 minutes, 5 minutes, 10 minutes, 15 minutes, 30 minutes, 45 minutes, 1 hour, 2 hours, 3 hours, 5 hours, 10 hours, 12 hours, or 1 day after food intake by a subject.

Formulations provided herein can include those suitable for oral including buccal and sub-lingual, intranasal, topical, transdermal, transdermal patch, pulmonary, vaginal, rectal, suppository, mucosal, systemic, or parenteral including intramuscular, intraarterial, intrathecal, intradermal, intraperitoneal, subcutaneous, and intravenous administration or in a form suitable for administration by aerosolization, inhalation or insufflation.

A composition can include carriers and excipients (including but not limited to buffers, carbohydrates, lipids, mannitol, proteins, polypeptides or amino acids such as glycine, antioxidants, bacteriostats, chelating agents, suspending agents, thickening agents and/or preservatives), metals (e.g., iron, calcium), salts, vitamins, minerals, water, oils including those of petroleum, animal, vegetable or synthetic origin, such as peanut oil, soybean oil, mineral oil, sesame oil and the like, saline solutions, aqueous dextrose and glycerol solutions, flavoring agents, coloring agents, detackifiers and other acceptable additives, adjuvants, or binders, other pharmaceutically acceptable auxiliary substances as required to approximate physiological conditions, such as pH buffering agents, tonicity adjusting agents, emulsifying agents, wetting agents and the like. Examples of excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

Non-limiting examples of pharmaceutically-acceptable excipients suitable for use in the disclosure include granulating agents, binding agents, lubricating agents, disintegrating agents, sweetening agents, glidants, anti-adherents, anti-static agents, surfactants, anti-oxidants, gums, coating agents, coloring agents, flavouring agents, dispersion enhancer, disintegrant, coating agents, plasticizers, preservatives, suspending agents, emulsifying agents, plant cellulosic material and spheronization agents, fillers, bulking agents, lubricant, and any combination thereof.

Non-limiting examples of pharmaceutically-acceptable excipients can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), each of which is incorporated by reference in its entirety.

A composition can be substantially free of preservatives. In some applications, the composition may contain at least one preservative.

A composition can be encapsulated within a suitable vehicle, for example, a liposome, a microspheres, or a microparticle. Alternatively, the compound can be incorporated and the microspheres, or composite of microspheres, and implanted for slow release over a period of time ranging from days to months.

The encapsulated mixture may be stable while refrigerated for at least about 1 week, 2 weeks, 3 weeks, 1 month, 2 months, 3 months, 4 months, or 5 months at 1° C., 2° C., 3° C., 4° C., or 5° C. The mixture may be stable for at least about 1 week, 2 weeks, 3 weeks, 1 month, 2 months, 3 months, 4 months, 5 months, 6 months, 7 months, 8 months, 9 months, 10 months, 15 months, 20 months, or 30 months at a temperature of 4° C.

The composition may comprise at least about 10⁸ active cells/g of one or more microbes in the population of isolated and purified microbes. The composition may comprise at least about 1.0×10⁸ active cells/g. The composition may comprise an amount of at least about 0.1%, 1%, 3%, 4%, 5%, 10%, 15%, 20%, 25%, 30%, 35%, 40%, 50%, or 75% viable microbes. The composition may comprise an amount of at least 1% to 5% viable obligate anaerobes. The composition may comprise an amount of at least 5% to 75% viable obligate anaerobes. The composition may comprise an amount of at least 10% to 50% viable obligate anaerobes.

A composition can be formulated as a sterile solution or suspension. The therapeutic or cosmetic compositions can be sterilized by conventional techniques or may be sterile filtered. The resulting aqueous solutions may be packaged for use as is, or lyophilized. The lyophilized preparation of the microbial composition can be packaged in a suitable form for oral administration, for example, capsule or pill.

The compositions can be administered topically and can be formulated into a variety of topically administrable compositions, such as solutions, suspensions, lotions, gels, pastes, medicated sticks, balms, creams, and ointments. Such compositions can contain solubilizers, stabilizers, tonicity enhancing agents, buffers and preservatives.

The compositions can also be formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, PEG, and the like. In suppository forms of the compositions, a low-melting wax such as a mixture of fatty acid glycerides, optionally in combination with cocoa butter, can be used.

In practicing the methods of treatment or use provided herein, therapeutically-effective amounts of the microbial compositions described herein are administered in compositions to a subject having a disease or condition to be treated. In some embodiments, the subject is a mammal such as a human. A therapeutically-effective amount can vary widely depending on the severity of the disease, the age and relative health of the subject, potency of the formulation, and other factors. Subjects can be, for example, humans, elderly adults, adults, adolescents, pre-adolescents, children, toddlers, infants, or neonates. A subject can be a patient. A subject can be an individual enrolled in a clinical study. A subject can be a laboratory animal, for example, a mammal, or a rodent.

The compositions can be formulated using one or more physiologically-acceptable carriers comprising excipients and auxiliaries, which facilitate processing of the microorganisms into preparations that can be used pharmaceutically. The formulation can be modified depending upon the route of administration chosen. The compositions described herein can be manufactured in a conventional manner, for example, by means of conventional mixing, dissolving, granulating, vitrification, spray-drying, lyophilizing, dragee-making, levigating, encapsulating, entrapping, emulsifying or compression processes.

In some embodiments, the composition is prepared in a dry form, for example, by spray-drying or lyophilization. In some embodiments, the formulation is prepared as a liquid capsule to maintain the liquid form of the microbes.

The composition may remain stable for extended periods of time. For example, the composition may remain stable (e.g., retain a therapeutically effective amount of one or more isolated and purified obligate anaerobes) after being stored at 4 degrees C.elsisus for 14 days or more. Alternately, in some instances, the composition may remain stable when stored at room temperature for 14 days or more. In various embodiments, the composition may be stable for at least about 1 day, 2, days, 3 days, 4 days, 5 days, 6 days, 7 days, 8 days, 9, days, 10 days, 11 days, 12 days, 13 days, 14 days, 15 days, 16 days, 17 days, 19 days, 20 days, 21 days, 22 days, 23 days, 24 days, 25 days, 26 days, 27 days, 28 days, 29 days, 30 days, 1 month, 2 months, 3 months, 4 months, 5 months, 6 months, 7 months, 8 months, 9 months, 10 months, 11 months, 12 months, 1 year, 2 years, 3 years, 4 years, or 5 years. The composition may be stable for at least at least about 1 day, 2, days, 3 days, 4 days, 5 days, 6 days, 7 days, 8 days, 9, days, 10 days, 11 days, 12 days, 13 days, 14 days, 15 days, 16 days, 17 days, 19 days, 20 days, 21 days, 22 days, 23 days, 24 days, 25 days, 26 days, 27 days, 28 days, 29 days, 30 days, 1 month, 2 months, 3 months, 4 months, 5 months, 6 months, 7 months, 8 months, 9 months, 10 months, 11 months, 12 months, 1 year, 2 years, 3 years, 4 years, or 5 years when stored at about 4° C. The composition may remain stable for at least about 1 day, 2, days, 3 days, 4 days, 5 days, 6 days, 7 days, 8 days, 9, days, 10 days, 11 days, 12 days, 13 days, 14 days, 15 days, 16 days, 17 days, 19 days, 20 days, 21 days, 22 days, 23 days, 24 days, 25 days, 26 days, 27 days, 28 days, 29 days, 30 days, 1 month, 2 months, 3 months, 4 months, 5 months, 6 months, 7 months, 8 months, 9 months, 10 months, 11 months, 12 months, 1 year, 2 years, 3 years, 4 years, or 5 years when stored under ambient temperature and pressure (about 20° C. to about 30° C., about 1 bar pressure).

Flow cytometry using live dead stain (as discussed for FIG. 4) can allow for the count of viable cells. This can then be done for samples at time point 0 and then compared to subsequent time points where samples are stored under diverse conditions (e.g. temperature, oxygen exposure, humidity, etc.).

Compositions provided herein can be stored at any suitable temperature. The formulation can be stored in cold storage, for example, at a temperature of about −80° C., about −20° C., about −4° C., or about 4° C. The storage temperature can be, for example, about 0° C., about 1° C., about 2° C., about 3° C., about 4° C., about 5° C., about 6° C., about 7° C., about 8° C., about 9° C., about 10° C., about 12° C., about 14° C., about 16° C., about 20° C., about 22° C., or about 25° C. In some embodiments, the storage temperature is between about 2° C. to about 8° C. Storage of microbial compositions at low temperatures, for example from about 2° C. to about 8° C., can keep the microbes alive and increase the efficiency of the composition, for example, when present in a liquid or gel formulation. Storage at freezing temperature, below 0° C., with a cryoprotectant can further extend stability.

The pH of the composition can range from about 3 to about 12. The pH of the composition can be, for example, from about 3 to about 4, from about 4 to about 5, from about 5 to about 6, from about 6 to about 7, from about 7 to about 8, from about 8 to about 9, from about 9 to about 10, from about 10 to about 11, or from about 11 to about 12 pH units. The pH of the composition can be, for example, about 3, about 4, about 5, about 6, about 7, about 8, about 9, about 10, about 11, or about 12 pH units. The pH of the composition can be, for example, at least 3, at least 4, at least 5, at least 6, at least 7, at least 8, at least 9, at least 10, at least 11 or at least 12 pH units. The pH of the composition can be, for example, at most 3, at most 4, at most 5, at most 6, at most 7, at most 8, at most 9, at most 10, at most 11, or at most 12 pH units. If the pH is outside the range desired by the formulator, the pH can be adjusted by using sufficient pharmaceutically-acceptable acids and bases. In some embodiments, the pH of the composition is between about 4 and about 6.

The compositions containing microbes described herein can be administered for prophylactic and/or therapeutic treatments. In therapeutic applications, the compositions can be administered to a subject already suffering from a disease or condition, in an amount sufficient to cure or at least partially arrest the symptoms of the disease or condition, or to cure, heal, improve, or ameliorate the condition. Microbial compositions can also be administered to lessen a likelihood of developing, contracting, or worsening a condition. Amounts effective for this use can vary based on the severity and course of the disease or condition, previous therapy, the subject's health status, weight, and response to the drugs, and the judgment of the treating physician.

Multiple therapeutic agents can be administered in any order or simultaneously. If simultaneously, the multiple therapeutic agents can be provided in a single, unified form, or in multiple forms, for example, as multiple separate pills. The composition can be packed together or separately, in a single package or in a plurality of packages. One or all of the therapeutic agents can be given in multiple doses. If not simultaneous, the timing between the multiple doses may vary to as much as about a month.

Compositions described herein can be administered before, during, or after the occurrence of a disease or condition, and the timing of administering the composition can vary. For example, the microbial composition can be used as a prophylactic and can be administered continuously to subjects with a propensity to conditions or diseases in order to lessen a likelihood of the occurrence of the disease or condition. The microbial compositions can be administered to a subject during or as soon as possible after the onset of the symptoms. The administration of the microbial compositions can be initiated within the first 48 hours of the onset of the symptoms, within the first 24 hours of the onset of the symptoms, within the first 6 hours of the onset of the symptoms, or within 3 hours of the onset of the symptoms. The initial administration can be via any route practical, such as by any route described herein using any formulation described herein. A microbial composition can be administered as soon as is practicable after the onset of a disease or condition is detected or suspected, and for a length of time necessary for the treatment of the disease, such as, for example, from about 1 month to about 3 months. The length of treatment can vary for each subject.

Compositions of the disclosure can be administered in combination with another therapy, for example, immunotherapy, chemotherapy, radiotherapy, anti-inflammatory agents, anti-viral agents, anti-microbial agents, and anti-fungal agents.

Compositions of the disclosure can be packaged as a kit. In some embodiments, a kit includes written instructions on the administration/use of the composition. The written material can be, for example, a label. The written material can suggest conditions methods of administration. The instructions provide the subject and the supervising physician with the best guidance for achieving the optimal clinical outcome from the administration of the therapy. The written material can be a label. In some embodiments, the label can be approved by a regulatory agency, for example the U.S. Food and Drug Administration (FDA), the European Medicines Agency (EMA), or other regulatory agencies.

For example, the composition is formulated for administration via pH-dependent release delivery, microbially-triggered delivery, time-controlled delivery, osmotically-regulated delivery, pressure-controlled delivery, multi matrix systems delivery, bioadhesion delivery, or multiparticulate delivery. The composition can also be formulated for release in the small or large intestine, colon, rectum, stomach, anus, or esophagus.

Compositions of the disclosure can be manufactured using a just-in-time (JIT) process. Just-in-time manufacturing, or lean manufacturing, can comprise manufacturing of the composition in response to an increase in demand for the composition. In some instances, the compositon is manufactured 1 hour, 2 hours, 3 hours, 4 hours, 5 hours, 10 hours, 15 hours, 20 hours, 1 day, 2 days, 3 days, 4 days, 5 days, 6 days, 1 week, 2 weeks, 3 weeks, 1 month, 2 months, 3 months, or 4 months prior to administration to an individual.

### Dosing

The appropriate quantity of a therapeutic or cosmetic composition to be administered, the number of treatments, and unit dose can vary according to a subject and/or the disease state of the subject.

The compositions described herein can be in unit dosage forms suitable for single administration of precise dosages. In unit dosage form, the formulation can be divided into unit doses containing appropriate quantities of one or more microbial compositions. The unit dosage can be in the form of a package containing discrete quantities of the formulation. Non-limiting examples are liquids in vials or ampoules. Aqueous suspension compositions can be packaged in single-dose non-reclosable containers. The composition can be in a multi-dose format. Multiple-dose reclosable containers can be used, for example, in combination with a preservative. Formulations for parenteral injection can be presented in unit dosage form, for example, in ampoules, or in multi-dose containers with a preservative.

The dosage can be in the form of a solid, semi-solid, or liquid composition. Non-limiting examples of dosage forms suitable for use in the disclosure include feed, food, pellet, lozenge, liquid, elixir, aerosol, inhalant, spray, powder, tablet, pill, capsule, gel, geltab, nanosuspension, nanoparticle, microgel, suppository troches, aqueous or oily suspensions, ointment, patch, lotion, dentifrice, emulsion, creams, drops, dispersible powders or granules, emulsion in hard or soft gel capsules, syrups, phytoceuticals, nutraceuticals, dietary supplement, and any combination thereof.

Described herein are compositions contain and methods for producing highly concentrated microbes. The concentration of a microbe can be for example, from about 10¹ to about 10¹⁸ colony forming units (CFU) per unit dosage form. The concentration of a microbe can be, for example, at least 10¹, at least 10², at least 10³, at least 10⁴, at least 10⁵, at least 10⁶, at least 10⁷, at least 10⁸, at least 10⁹, at least 10¹⁰, at least 10¹¹, at least 10¹², at least 10¹³, at least 10¹⁴, at least 10¹⁵, at least 10¹⁶, at least 10¹⁷, or at least 10¹⁸ CFU per unit dosage form. The concentration of a microbe can be, for example, at most 10¹, at most 10², at most 10³, at most 10⁴, at most 10⁵, at most 10⁶, at most 10⁷, at most 10⁸, at most 10⁹, at most 10¹⁰, at most 10¹¹, at most 10¹², at most 10¹³, at most 10¹⁴, at most 10¹⁵, at most 10¹⁶, at most 10¹⁷, or at most 10¹⁸ CFU per unit dosage form. One or more microbes may be present in a single unit dosage form in amounts of about 10⁸ CFU to about 10¹¹ CFU. In some embodiments, the amount of one or more microbes present in a unit dosage form is at least about 10⁸ CFU. In some embodiments, an amount of a microbes present in a unit dosage form is at least about 10⁹ CFU.

A unit dosage form of the composition described herein may comprise one or more highly concentrated microbes. The quantity of a microbe can be for example, from about 10⁶ to about 10¹¹ microbes/gram. The concentration of a microbe can be, for example, at least 10¹, at least 10², at least 10³, at least 10⁴, at least 10⁵, at least 10⁶, at least 10⁷, at least 10⁸, at least 10⁹, at least 10¹⁰, at least 10¹¹, at least 10¹², at least 10¹³, at least 10¹⁴, at least 10¹⁵, at least 10¹⁶, at least 10¹⁷, or at least 10¹⁸ microbes per gram. The concentration of a microbe can be, for example, at most 10¹, at most 10², at most 10³, at most 10⁴, at most 10⁵, at most 10⁶, at most 10⁷, at most 10⁸, at most 10⁹, at most 10¹⁰, at most 10¹¹, at most 10¹², at most 10¹³, at most 10¹⁴, at most 10¹⁵, at most 10¹⁶, at most 10¹⁷, or at most 10¹⁸ per gram.

A unit dosage form of the composition may comprise one or more highly concentrated microbes that are active cells. An active cell can be a viable microbe. A unit dosage form may comprise from about 10⁹ to about 10¹¹ active cells/gram. A unit dosage form may comprise at least 10¹, at least 10², at least 10³, at least 10⁴, at least 10⁵, at least 10⁶, at least 10⁷, at least 10⁸, at least 10⁹, at least 10¹⁰, at least 10¹¹, at least 10¹², at least 10¹³, at least 10¹⁴, at least 10¹⁵, at least 10¹⁶, at least 10¹⁷, or at least 10¹⁸ active cells per gram. A unit dosage form may comprise at most 10¹, at most 10², at most 10³, at most 10⁴, at most 10⁵, at most 10⁶, at most 10⁷, at most 10⁸, at most 10⁹, at most 10¹⁰, at most 10¹¹, at most 10¹², at most 10¹³, at most 10¹⁴, at most 10¹⁵, at most 10¹⁶, at most 10¹⁷, or at most 10¹⁸ per gram.

The compositions disclosed herein can be administered in one or more dose units. The compositions comprising one or more highly concentrated microbial strains may be administered in fewer unit dosage forms than would otherwise be required if administering formulations comprising non-concentrated microbes. Administration of a composition in higher concentrations and fewer dosage forms may improve ease of administration to a subject and enhance therapeutic outcomes. As a non-limiting example, a composition may be administered in 10 or less unit dosage forms, in a single administration. A composition may be administered in 1 unit dosage form to 5 unit dosage forms, in a single administration. A composition may be administered in a single unit dosage form, per administration. A composition may be administered in two or three unit dosage forms, per administration.

The compositions described herein can be formulated with any suitable therapeutically-effective concentration of prebiotic. For example, the therapeutically-effective concentration of a prebiotic can be at least about 1 mg/ml, about 2 mg/ml, about 3 mg/ml, about 4 mg/ml, about 5 mg/ml, about 10 mg/ml, about 15 mg/ml, about 20 mg/ml, about 25 mg/ml, about 30 mg/ml, about 35 mg/ml, about 40 mg/ml, about 45 mg/ml, about 50 mg/ml, about 55 mg/ml, about 60 mg/ml, about 65 mg/ml, about 70 mg/ml, about 75 mg/ml, about 80 mg/ml, about 85 mg/ml, about 90 mg/ml, about 95 mg/ml, about 100 mg/ml, about 110 mg/ml, about 125 mg/ml, about 130 mg/ml, about 140 mg/ml, or about 150 mg/ml. For example, the therapeutically-effective concentration of a prebiotic can be at most about 1 mg/ml, about 2 mg/ml, about 3 mg/ml, about 4 mg/ml, about 5 mg/ml, about 10 mg/ml, about 15 mg/ml, about 20 mg/ml, about 25 mg/ml, about 30 mg/ml, about 35 mg/ml, about 40 mg/ml, about 45 mg/ml, about 50 mg/ml, about 55 mg/ml, about 60 mg/ml, about 65 mg/ml, about 70 mg/ml, about 75 mg/ml, about 80 mg/ml, about 85 mg/ml, about 90 mg/ml, about 95 mg/ml, about 100 mg/ml, about 110 mg/ml, about 125 mg/ml, about 130 mg/ml, about 140 mg/ml, or about 150 mg/ml. For example, the therapeutically-effective concentration of a prebiotic can be about 1 mg/ml, about 2 mg/ml, about 3 mg/ml, about 4 mg/ml, about 5 mg/ml, about 10 mg/ml, about 15 mg/ml, about 20 mg/ml, about 25 mg/ml, about 30 mg/ml, about 35 mg/ml, about 40 mg/ml, about 45 mg/ml, about 50 mg/ml, about 55 mg/ml, about 60 mg/ml, about 65 mg/ml, about 70 mg/ml, about 75 mg/ml, about 80 mg/ml, about 85 mg/ml, about 90 mg/ml, about 95 mg/ml, about 100 mg/ml, about 110 mg/ml, about 125 mg/ml, about 130 mg/ml, about 140 mg/ml, or about 150 mg/ml. In some embodiments, the concentration of a prebiotic in a composition is about 70 mg/ml. In some embodiments, the prebiotic is inulin.

The compositions of the disclosure can be administered, for example, 1, 2, 3, 4, 5, or more times daily. The compositions of the disclosure can be administered, for example, daily, every other day, three times a week, twice a week, once a week, or at other appropriate intervals for treatment of the condition.

A composition may be administered daily, where 5 or less unit dosage forms are administered to a subject. A composition may be administered twice daily where 5 or fewer unit dosage forms are administered to a subject. Ten, 8, 6, 5, 4, 3, or 2 unit dosage forms or less may be administered to a subject daily.

### Role of Butyrate

Short chain fatty acids (SCFAs) such as butyrate can play a central role in modulating various body functions. For example, butyrate can protect the brain and enhance plasticity in neurological diseases. Butyrate can serve an anti-inflammatory factor. Butyrate can affect gut permeability. Low levels of butyrate producing microbes (e.g. Clostridium clusters XIVa and IV) and/or reduced lactate producing bacteria (e.g. Bifidobacterium adolescentis) can be correlated with, for example, gut dysbiosis, skin disorders, metabolic disorders, and behavioral/neurological disorders. Subsets of a formulation that comprise at least one primary fermenter and at least one secondary fermenter can be used for the treatment and/or mitigate progression of a disorder or condition.

In the colon, dietary fiber can be processed by butyrate-producing microorganisms to produce butyrate (i.e. butanoate), which is an SCFA. In turn, butyrate can initiate G-protein coupled receptor (GPCR) signaling, leading to, for example, glucagon-like peptide-1 (GLP-1) secretion. GLP-1 can result in increased insulin sensitivity. Alteration of butyrate-producing microbiome in a subject can be associated with a disorder.

In some embodiments, the composition comprises a microbe with a butyrate kinase. Butyrate kinase is an enzyme that can belong to a family of transferases, for example those transferring phosphorus-containing groups (e.g., phosphotransferases) with a carboxy group as acceptor. The systematic name of this enzyme class can be ATP:butanoate 1-phosphotransferase. Butyrate kinase can participate in butyrate metabolism. Butyrate kinase can catalyze the following reaction:

ADP+butyryl-phosphate [symbol] ATP+butyrate

In some embodiments, the composition comprises a microbe with a Butyrate-Coenzyme A. Butyrate-Coenzyme A, also butyryl-coenzyme A, can be a coenzyme A-activated form of butyric acid. It can be acted upon by butyryl-CoA dehydrogenase and can be an intermediary compound in acetone-butanol-ethanol fermentation. Butyrate-Coenzyme A can be involved in butyrate metabolism.

In some embodiments, the composition comprises a microbe with a Butyrate-Coenzyme A transferase. Butyrate-Coenzyme A transferase, also known as butyrate-acetoacetate CoA-transferase, can belong to a family of transferases, for example, the CoA-transferases. The systematic name of this enzyme class can be butanoyl-CoA:acetoacetate CoA-transferase. Other names in common use can include butyryl coenzyme A-acetoacetate coenzyme A-transferase, and butyryl-CoA-acetoacetate CoA-transferase. Butyrate-Coenzyme A transferase can catalyze the following chemical reaction:

butanoyl-CoA+acetoacetate [symbol] butanoate+acetoacetyl-CoA

In some embodiments, the composition comprises a microbe with a Butyryl-Coenzyme A dehydrogenase. Butyryl-CoA dehydrogenase can belong to the family of oxidoreductases, for example, those acting on the CH—CH group of donor with other acceptors. The systematic name of this enzyme class can be butanoyl-CoA: acceptor 2,3-oxidoreductase. Other names in common use can include butyryl dehydrogenase, unsaturated acyl-CoA reductase, ethylene reductase, enoyl-coenzyme A reductase, unsaturated acyl coenzyme A reductase, butyryl coenzyme A dehydrogenase, short-chain acyl CoA dehydrogenase, short-chain acyl-coenzyme A dehydrogenase, 3-hydroxyacyl CoA reductase, and butanoyl-CoA:(acceptor) 2,3-oxidoreductase. Non-limiting examples of metabolic pathways that butyryl-CoA dehydrogenase can participate in include: fatty acid metabolism; valine, leucine and isoleucine degradation; and butanoate metabolism. Butyryl-CoA dehydrogenase can employ one cofactor, FAD. Butyryl-CoA dehydrogenase can catalyze the following reaction:

butyryl-CoA+acceptor [symbol] 2-butenoyl-CoA+reduced acceptor

In some embodiments, the composition comprises a microbe with a beta-hydroxybutyryl-CoA dehydrogenase. Beta-hydroxybutyryl-CoA dehydrogenase or 3-hydroxybutyryl-CoA dehydrogenase can belong to a family of oxidoreductases, for example, those acting on the CH—OH group of donor with NAD+ or NADP+ as acceptor. The systematic name of the enzyme class can be (S)-3-hydroxybutanoyl-CoA:NADP+ oxidoreductase. Other names in common use can include beta-hydroxybutyryl coenzyme A dehydrogenase, L(+)-3-hydroxybutyryl-CoA dehydrogenase, BHBD, dehydrogenase, L-3-hydroxybutyryl coenzyme A (nicotinamide adenine, dinucleotide phosphate), L-(+)-3-hydroxybutyryl-CoA dehydrogenase, and 3-hydroxybutyryl-CoA dehydrogenase. Beta-hydroxybutyryl-CoA dehydrogenase enzyme can participate in benzoate degradation via co-ligation. Beta-hydroxybutyryl-CoA dehydrogenase enzyme can participate in butanoate metabolism. Beta-hydroxybutyryl-CoA dehydrogenase can catalyze the following reaction:

(S)-3-hydroxybutanoyl-CoA+NADP⁺ [symbol] 3-acetoacetyl-CoA+NADPH+H⁺

In some embodiments, the composition comprises a microbe with a crotonase. Crotonase can comprise enzymes with, for example, dehalogenase, hydratase, isomerase activities. Crotonase can be implicated in carbon-carbon bond formation, cleavage, and hydrolysis of thioesters. Enzymes in the crotonase superfamily can include, for example, enoyl-CoA hydratase which can catalyse the hydratation of 2-trans-enoyl-CoA into 3-hydroxyacyl-CoA; 3-2trans-enoyl-CoA isomerase or dodecenoyl-CoA isomerise (e.g., EC 5.3.3.8), which can shift the 3-double bond of the intermediates of unsaturated fatty acid oxidation to the 2-trans position; 3-hydroxbutyryl-CoA dehydratase (e.g., crotonase; EC 4.2.1.55), which can be involved in the butyrate/butanol-producing pathway; 4-Chlorobenzoyl-CoA dehalogenase (e.g., EC 3.8.1.6) which can catalyze the conversion of 4-chlorobenzoate-CoA to 4-hydroxybenzoate-CoA; dienoyl-CoA isomerase, which can catalyze the isomerisation of 3-trans,5-cis-dienoyl-CoA to 2-trans,4-trans-dienoyl-CoA; naphthoate synthase (e.g., MenB, or DHNA synthetase; EC 4.1.3.36), which can be involved in the biosynthesis of menaquinone (e.g., vitamin K2); carnitine racemase (e.g., gene caiD), which can catalyze the reversible conversion of crotonobetaine to L-carnitine in Escherichia coli; Methylmalonyl CoA decarboxylase (e.g., MMCD; EC 4.1.1.41); carboxymethylproline synthase (e.g., CarB), which can be involved in carbapenem biosynthesis; 6-oxo camphor hydrolase, which can catalyze the desymmetrization of bicyclic beta-diketones to optically active keto acids; the alpha subunit of fatty acid oxidation complex, a multi-enzyme complex that can catalyze the last three reactions in the fatty acid beta-oxidation cycle; and AUH protein, which can be a bifunctional RNA-binding homologue of enoyl-CoA hydratase.

In some embodiments, the composition comprises a microbe with a thiolase. Thiolases, also known as acetyl-coenzyme A acetyltransferases (ACAT), can convert two units of acetyl-CoA to acetoacetyl CoA, for example, in the mevalonate pathway. Thiolases can include, for example, degradative thiolases (e.g., EC 2.3.1.16) and biosynthetic thiolases (e.g., EC 2.3.1.9). 3-ketoacyl-CoA thiolase, also called thiolase I, can be involved in degradative pathways such as fatty acid beta-oxidation. Acetoacetyl-CoA thiolase, also called thiolase II, can be specific for the thiolysis of acetoacetyl-CoA and can be involved in biosynthetic pathways such as poly beta-hydroxybutyric acid synthesis or steroid biogenesis. A thiolase can catalyze the following reaction:

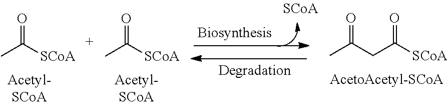

Production of butyrate can involve two major phases or microbes, for example, a primary fermenter and a secondary fermenter. The primary fermenter can produce intermediate molecules (e.g. lactate, acetate) when given an energy source (e.g. fiber). The secondary fermenter can convert the intermediate molecules produced by the primary fermenter into butyrate. Non-limiting examples of primary fermenter include Akkermansia muciniphila, Bifidobacterium adolescentis, Bifidobacterium infantis and Bifidobacterium longum. Non-limiting examples of secondary fermenter include Clostridium beijerinckii, Clostridium butyricum, Clostridium indolis, Eubacterium hallii, and Faecalibacterium prausnitzii. A combination of primary and secondary fermenters can be used to produce butyrate in a subject. Subsets of a formulation that comprises at least one primary fermenter and at least one secondary fermenter can be used for the treatment and/or mitigate progression of a metabolic health condition. The formulation can additionally comprise a prebiotic.

In some embodiments, a therapeutic composition comprises at least one primary fermenter and at least one secondary fermenter. In some embodiments, a therapeutic composition comprises at least one primary fermenter, at least one secondary fermenter, and at least one prebiotic. In one non-limiting example, a therapeutic composition can comprise Bifidobacterium adolescentis, Clostridium indolis, and inulin. In another non-limiting example, a therapeutic composition can comprise Bifidobacterium longum, Faecalibacterium prausnitzii, and starch. In one non-limiting example, a therapeutic composition can comprise Akkermansia muciniphila, Bifidobacterium infantis, Clostridium beijerinckii, Clostridium butyricum, Eubacterium hallii, and inulin. In one non-limiting example, a therapeutic composition can comprise Clostridium beijerinckii, Clostridium butyricum, Bifidobacterium infantis, and inulin. In one non-limiting example, a therapeutic composition can comprise Akkermansia muciniphila, Clostridium beijerinckii, Clostridium butyricum, Eubacterium hallii, Bifidobacterium infantis, and inulin.

Alterations in the relative abundance of SCFAs relative to each other can lead to a disorder. For example, altered fiber to acetate production pathway or acetate to butyrate production pathway can lead to metabolic disorders such as bloating.

Akkermansia muciniphila can be a gram negative, strict anaerobe that can play a role in mucin degradation. Akkermansia muciniphila can be associated with increased levels of endocannabinoids that control inflammation, the gut barrier, and gut peptide secretion. Akkermansia muciniphila can serve as a primary fermenter.

Bifidobacterium adolescentis can be a gram-positive anaerobe, which can be found in healthy human gut from infancy. Bifidobacterium adolescentis can synthesize B vitamins. Bifidobacterium adolescentis can serve as a primary fermenter.

Bifidobacterium infantis can be a gram-positive, catalase negative, micro-aerotolerant anaerobe. Bifidobacterium infantis can serve as a primary fermenter.

Bifidobacterium longum can be a gram-positive, catalase negative, micro-aerotolerant anaerobe. Bifidobacterium longum can serve as a primary fermenter.

Clostridium beijerinckii can be a gram-positive, strict anaerobe that belongs to Clostridial cluster I. Clostridium beijerinckii can serve as a secondary fermenter.

Clostridium butyricum can be a gram-positive, strict anaerobe that can serve as a secondary fermenter.

Clostridium indolis can be a gram-positive, strict anaerobe that belongs to Clostridial cluster XIVA. Clostridium indolis can serve as a secondary fermenter.

Eubacterium hallii can be a gram-positive, anaerobe that belongs to Arrangement A Clostridial cluster XIVA. Eubacterium hallii can serve as a secondary fermenter.

Faecalibacterium prausnitzii can be a gram-positive, anaerobe belonging to Clostridial cluster IV. Faecalibacterium prausnitzii can be one of the most common gut bacteria and the largest butyrate producer. Faecalibacterium prausnitzii can serve as a secondary fermenter.

Non-limiting examples of genes and/or proteins involved in the generation of butyrate include: butyryl-CoA dehydrogenase, beta-hydroxybutyryl-CoA dehydrogenase or 3-hydroxybutyryl-CoA dehydrogenase, crotonase, electron transfer protein a, electron transfer protein b, and thiolase. In some embodiments, the composition comprises a microbe with a gene or protein involved in SCFA (e.g., butyrate) production.

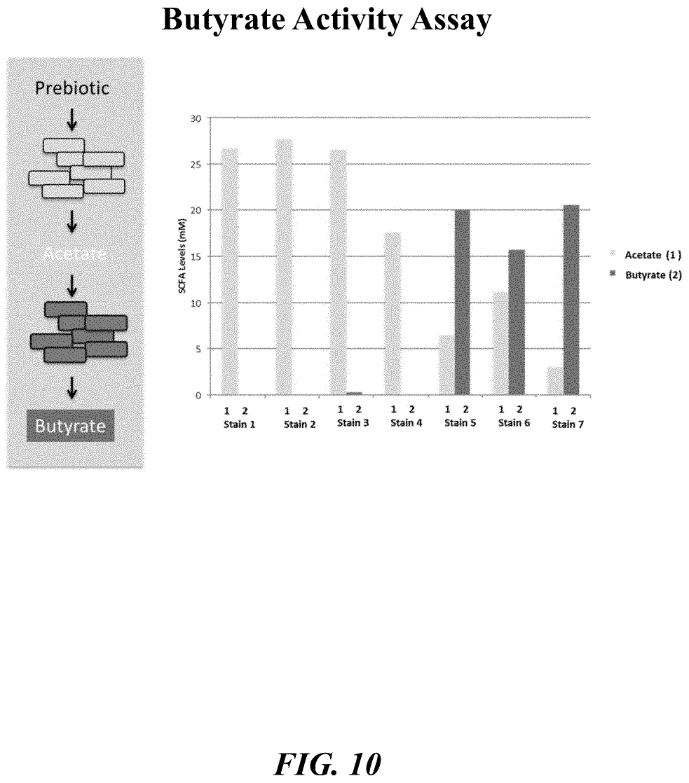

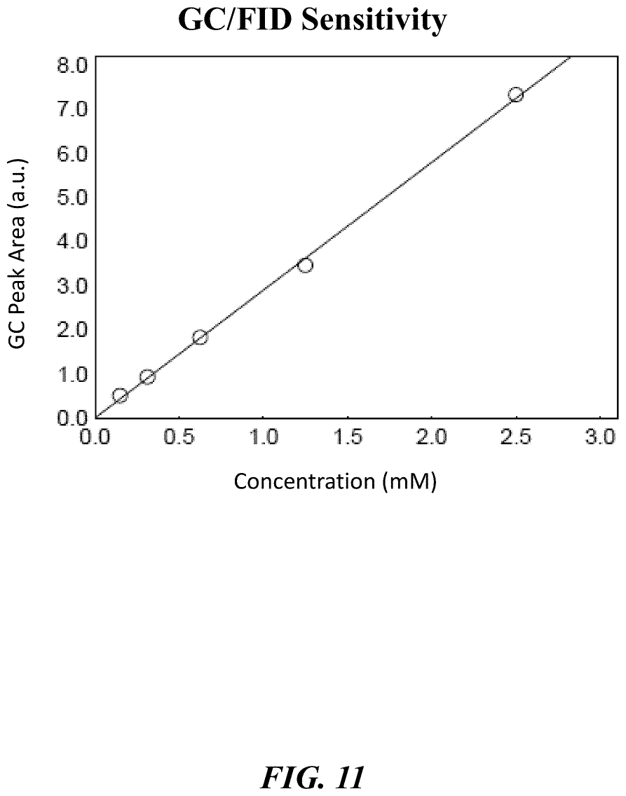

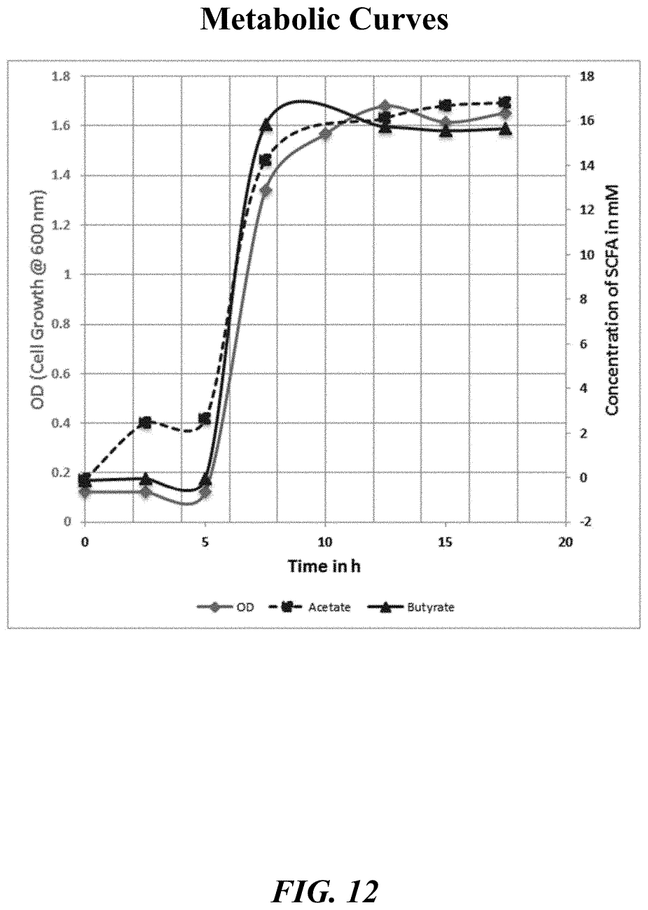

FIG. 10 shows the measurements of SCFAs for two metabolites, acetate and butyrate across seven strains. The formulations may be formulated to allow for various amounts of primary and secondary fermenters. FIG. 11 uses gas chromatography with a flame ionization detector (GC/FID) to relate GC peak area (a.u.) to the microbial concentration (mM). In fact, concentrations of at least about 0.010 mM, 0.015 mM, 0.020 mM, 0.025 mM, 0.030 mM, 0.04 mM, 0.05 mM, 0.1 mM, 0.15 mM, 0.20 mM, 0.25 mM, 0.30 mM, 0.35 mM, or 0.40 mM may be reliably measured. As shown in FIG. 12, GC/FID (gas chromatography with a flame ionization detector) can be used to measure the metabolic activity of the microbial cells across time to monitor high throughput production of short chain fatty acids. The metabolic activity assay (MAC assay) in FIG. 12 can also be used to determine the metabolic activity of the microbial cells. The two metabolites can be acetate and butyrate. This assay uses a gas chromatography (GC) instrument with a flame ionization detector (FID) and a specific column designed to detect various short chain fatty acids.

### Biological Samples

A biological sample can be collected from a subject to determine the microbiome profile of the subject. The biological sample can be any sample type from any microbial habitat on the body of a subject. Non-limiting examples of microbial habitats include skin habitat, umbilical habitat, vaginal habitat, amniotic fluid habitat, conjunctival habitat, intestinal habitat, stomach habitat, gut habitat, oral habitat, nasal habitat, gastrointestinal tract habitat, respiratory habitat, and urogenital tract habitat.

Depending on the application, the selection of a biological sample can be tailored to the specific application. The biological sample can be for example, whole blood, serum, plasma, mucosa, saliva, cheek swab, urine, stool, cells, tissue, bodily fluid, lymph fluid, CNS fluid, and lesion exudates. A combination of biological samples can be used with the methods of the disclosure.

### Characterization of the Microbes in a Formulation and Microbiome

The microbes may be characterized in the formulation and confirmed for administration into the host. Sequencing the host microbiome can be used to develop formulations for the individual. Nucleic acid sample prepared from a biological sample can be subjected to a detection method to generate a profile of the microbiome associated with the sample. Profiling of a microbiome can comprise one or more detection methods.

Methods of the disclosure can be used to measure, for example, a 16S ribosomal subunit, a 23S ribosomal subunit, intergenic regions, and other genetic elements. Suitable detection methods can be chosen to provide sufficient discriminative power in a particular microbe in order to identify informative microbiome profiles.

In some applications, the entire genomic region of the 16S or 23S ribosomal subunit of the microbe is analyzed to determine a subject's microbiome profile. In some applications, the variable regions of the 16S and/or 23S ribosomal subunit of the microbe are analyzed to determine a subject's microbiome profile.

In some applications, the entire genome of the microbe is analyzed to determine a subject's microbiome profile. In other applications, the variable regions of the microbe's genome are analyzed to determine a subject's microbiome profile. For example, genetic variation in the genome can include restriction fragment length polymorphisms, single nucleotide polymorphisms, insertions, deletions, indels (insertions-deletions), microsatellite repeats, minisatellite repeats, short tandem repeats, transposable elements, randomly amplified polymorphic DNA, amplification fragment length polymorphism or a combination thereof.

In some embodiments, sequencing methods such as long-read length single molecule sequencing is used for detection. Long read sequencing can provide microbial classification down to the strain resolution of each microbe. Examples of sequencing technologies that can be used with the present disclosure for achieving long read lengths include the SMRT sequencing systems from Pacific Biosciences, long read length Sanger sequencing, long read ensemble sequencing approaches, e.g., Illumina/Moleculo sequencing and potentially, other single molecule sequencing approaches, such as Nanopore sequencing technologies.

Long read sequencing can include sequencing that provides a contiguous sequence read of for example, longer than 500 bases, longer than 800 bases, longer than 1000 bases, longer than 1500 bases, longer than 2000 bases, longer than 3000 bases, or longer than 4500 bases.

In some embodiments, detection methods of the disclosure comprise amplification-mode sequencing to profile the microbiome. In some embodiments, detection methods of the disclosure comprise a non-amplification mode, for example Whole Genome Shotgun (WGS) sequencing, to profile the microbiome.

Primers used in the disclosure can be prepared by any suitable method, for example, cloning of appropriate sequences and direct chemical synthesis. Primers can also be obtained from commercial sources. In addition, computer programs can be used to design primers. Primers can contain unique barcode identifiers.

Microbiome profiling can further comprise use of for example, a nucleic acid microarray, a biochip, a protein microarray, an analytical protein microarray, reverse phase protein microarray (RPA), a digital PCR device, and/or a droplet digital PCR device.

In some cases, sequencing of at least one target nucleic acid from at least two microbes in a composition or from a gut microbiome sample from an individual is performed. The sequencing can determine the quatity of the at least two microbes in the composition or from the gut microbiome sample. The target nucleic acid can be DNA or RNA. The target nucleic acid can be a species specific marker. The species specific marker can be a phylogenetically informative marker. Examples of phylogenetically informative markers include, but are not limited to, 5S rRNA, 16S rRNA, and 23S rRNA, internal transcribed spacer (ITS), cytochrome oxidase I (COI), cytochrome oxidase II (COII), and cytochrome b. In some cases, a barcode can be attached to the target nucleic acid or a nucleic acid comprising the target nucleic acid. The barcode can be attached prior to amplification. In some cases, following sequencing, algorithms are used to correct for errors introduced by amplification. Examples of algorithms to correct for errors introduced by amplification include, but are not limited to, DADA2. The sequencing can be used to determine the species of microbes in a gut microbiome sample from an individual. The sequencing can be used to determine the quantity of the at least two microbes in the composition or the gut microbiome sample from an individual. The combination of addition of barcodes to the target nucleic acid or a nucleic acid comprising the target nucleic acid in combination with the use of an algorithm to correct for errors introduced by amplification can reduce bias in the quantities of the at least two microbes detected.

In some instances, a simulator of the human intestinal microbial ecosystem (SHIME) is used. The SHIME can be used to: a) test the impact of the composition on the gut microbiome present in the individual, b) test survival of the microbes of the composition in a new environment or under a specific condition, c) test production of target metabolites under a specific condition, d) test impact of bile acids or other host specific metabolites on bacterial composition, and e) enrich for certain microbes via manipulation of the SHIME system. Specific conditions tested can include variations in pH and variation in prebiotics co-administered with the composition. Stable isotope probes can be used to measure metabolic activity of at least one microbe in the SHIME.

In some embodiments, the microbial profile is determined using additional information such as age, weight, gender, medical history, risk factors, family history, or any other clinically relevant information.

In some applications, a subject's microbiome profile comprises a single microbiome. For example, a subject's microbiome profile can comprise of at least one biological sample from only the subject's intestinal microbiome. In another example, a subject's microbiome profile can comprise of at least one biological sample from only the subject's stomach microbiome. In another example, a subject's microbiome profile can comprise of at least one biological sample from only the subject's gut microbiome. In another example, a subject's microbiome profile can comprise of at least one biological sample from only the subject's oral microbiome.

In some applications, a subject's microbiome profile comprises at least one biological sample from more than one microbiome. For example, a subject's microbiome profile can comprise of at least one biological sample from the subject's skin microbiome and at least one biological sample from the umbilical microbiome. In another example, a subject's microbiome profile can comprise of at least one biological sample from the subject's intestinal microbiome, at least one biological sample from the stomach microbiome, at least one biological sample from the gut microbiome, and at least one biological sample from the oral microbiome. In another example, a subject's microbiome profile can comprise of at least one biological sample from the subject's intestinal microbiome, and at least one biological sample from stomach microbiome. In another example, a subject's microbiome profile can comprise of at least one biological sample from the subject's gut microbiome, and at least one biological sample from oral microbiome. In some applications, a subject's microbiome profile can comprise of at least 2, 3, 4, 5, 6, 7, 8, 9, 10, 11, 12, 13, 14, 15, 16, 17, 18, 19, 20 microbiomes.

A subject's microbiome profile can comprise of one microbe. In some applications, a subject's microbiome profile comprises of, for example, 2 microbes, 3 or fewer microbes, 4 or fewer microbes, 5 or fewer microbes, 6 or fewer microbes, 7 or fewer microbes, 8 or fewer microbes, 9 or fewer microbes, 10 or fewer microbes, 11 or fewer microbes, no more than 12 microbes, 13 or fewer microbes, 14 or fewer microbes, 15 or fewer microbes, 16 or fewer microbes, 18 or fewer microbes, 19 or fewer microbes, 20 or fewer microbes, 25 or fewer microbes, 30 or fewer microbes, 35 or fewer microbes, 40 or fewer microbes, 45 or fewer microbes, 50 or fewer microbes, 55 or fewer microbes, 60 or fewer microbes, 65 or fewer microbes, 70 or fewer microbes, 75 or fewer microbes, 80 or fewer microbes, 85 or fewer microbes, 90 or fewer microbes, 100 or fewer microbes, 200 or fewer microbes, 300 or fewer microbes, 400 or fewer microbe, 500 or fewer microbes, 600 or fewer microbes, 700 or fewer microbes, or 800 or fewer microbes.

In some embodiments, provided are therapeutic compositions to treat microbiome-related health conditions and diseases for which microbiome therapeutics and diagnostics can be used. These health conditions can include: preterm labor, chronic fatigue syndrome, skin health (e.g. acne), Type 2 Diabetes Mellitus (T2DM), allergies, depression, autism, asthma, hypertension, irritable bowel syndrome and/or pain associated therewith, metabolism, obesity, drug metabolism, vaginosis, atopic dermatitis, psoriasis, Type I Diabetes (T1DM), Multiple Sclerosis, Clostridium Difficile, Inflammatory Bowel Disease (IBD), Crohn's Disease, genitourinary disorders, and heart disease.

The composition may comprise a purified microorganism population consisting of bacteria with at least about: 70%, 75%, 80%, 85%, 87%, 90%, 91%, 92%, 93%, 94%, 95%, 96%, 97%, 98%, 99%, 99.5%, or 100% sequence identity to the 16SrRNA and/or 23S rRNA of a microorganism selected from the group consisting of: Akkermansia muciniphila, Anaerostipes caccae, Bifidobacterium adolescentis, Bifidobacterium bifidum, Bifidobacterium infantis, Bifidobacterium longum, Butyrivibrio fibrisolvens, Clostridium acetobutylicum, Clostridium aminophilum, Clostridium beijerinckii, Clostridium butyricum, Clostridium colinum, Clostridium coccoides, Clostridium indolis, Clostridium nexile, Clostridium orbiscindens, Clostridium propionicum, Clostridium xylanolyticum, Enterococcus faecium, Eubacterium hallii, Eubacterium rectale, Faecalibacterium prausnitzii, Fibrobacter succinogenes, Lactobacillus acidophilus, Lactobacillus brevis, Lactobacillus bulgaricus, Lactobacillus casei, Lactobacillus caucasicus, Lactobacillus fermentum, Lactobacillus helveticus, Lactobacillus lactis, Lactobacillus plantarum, Lactobacillus reuteri, Lactobacillus rhamnosus, Oscillospira guilliermondii, Roseburia cecicola, Roseburia inulinivorans, Ruminococcus flavefaciens, Ruminococcus gnavus, Ruminococcus obeum, Stenotrophomonas nitritireducens, Streptococcus cremoris, Streptococcus faecium, Streptococcus infantis, Streptococcus mutans, Streptococcus thermophilus, Anaerofustis stercorihominis, Anaerostipes hadrus, Anaerotruncus colihominis, Clostridium sporogenes, Clostridium tetani, Coprococcus, Coprococcus eutactus, Eubacterium cylindroides, Eubacterium dolichum, Eubacterium ventriosum, Roseburia faeccis, Roseburia hominis, Roseburia intestinalis, Lacatobacillus bifidus, Lactobacillus johnsonii, Lactobacilli, Acidaminococcus fermentans, Acidaminococcus intestine, Blautia hydrogenotrophica, Citrobacter amalonaticus, Citrobacter freundii, Clostridium aminobutyricum Clostridium bartlettii, Clostridium cochlearium, Clostridium kluyveri, Clostridium limosum, Clostridium malenominatum, Clostridium pasteurianum, Clostridium peptidivorans, Clostridium saccharobutylicum, Clostridium sporosphaeroides, Clostridium sticklandii, Clostridium subterminale, Clostridium symbiosum, Clostridium tetanomorphum, Eubacterium oxidoreducens, Eubacterium pyruvativorans, Methanobrevibacter smithii, Morganella morganii, Peptomphilus asaccharolyticus, and Peptostreptococcus, and any combination thereof.

In some embodiments, provided are therapeutic compositions comprising an isolated and/or purified microorganism population consisting of bacteria with at least about: 70%, 75%, 80%, 85%, 87%, 90%, 91%, 92%, 93%, 94%, 95%, 96%, 97%, 98%, 99%, 99.5%, or 100% sequence identity to the 16SrRNA and/or 23S rRNA of a microorganism selected from the group consisting of: Akkermansia muciniphila, Bifidobacterium adolescentis, Bifidobacterium infantis, Bifidobacterium longum, Clostridium beijerinckii, Clostridium butyricum, Clostridium indolis, Eubacterium hallii, and any combination thereof.

In some embodiments, provided are therapeutic compositions comprising an isolated and/or purified microorganism population consisting of bacteria with at least about: 70%, 75%, 80%, 85%, 87%, 90%, 91%, 92%, 93%, 94%, 95%, 96%, 97%, 98%, 99%, 99.5%, or 100% sequence identity to the 16SrRNA and/or 23S rRNA of a microorganism selected from the group consisting of: Akkermansia muciniphila, Bifidobacterium adolescentis, Bifidobacterium infantis, Bifidobacterium longum, Clostridium beijerinckii, Clostridium butyricum, Clostridium indolis, Eubacterium hallii, Faecalibacterium prausnitzii, and any combination thereof.

In some embodiments, provided are therapeutic compositions comprising an isolated and/or purified microorganism population consisting of bacteria with at least about: 70%, 75%, 80%, 85%, 87%, 90%, 91%, 92%, 93%, 94%, 95%, 96%, 97%, 98%, 99%, 99.5%, or 100% sequence identity to the 16SrRNA and/or 23S rRNA of a microorganism selected from the group consisting of: Akkermansia muciniphila, Bifidobacterium infantis, Clostridium beijerinckii, Clostridium butyricum, Eubacterium hallii, and any combination thereof.

In some embodiments, provided are therapeutic compositions comprising an isolated and/or purified microorganism population consisting of bacteria with at least about: 70%, 75%, 80%, 85%, 87%, 90%, 91%, 92%, 93%, 94%, 95%, 96%, 97%, 98%, 99%, 99.5%, or 100% sequence identity to the 16SrRNA and/or 23S rRNA of a microorganism selected from the group consisting of: Clostridium beijerinckii, Clostridium butyricum, Bifidobacterium infantis, and any combination thereof.

In some embodiments, provided are therapeutic compositions comprising an isolated and/or purified microorganism population consisting of bacteria with at least about: 70%, 75%, 80%, 85%, 87%, 90%, 91%, 92%, 93%, 94%, 95%, 96%, 97%, 98%, 99%, 99.5%, or 100% sequence identity to the 16SrRNA and/or 23S rRNA of a microorganism selected from the group consisting of: Faecalibacterium prausnitzii, Clostridium beijerinckii, Bifidobacterium bifidum, and Lactobacillus brevis, and any combination thereof.

In some embodiments, provided are therapeutic compositions comprising an isolated and/or purified microorganism population consisting of bacteria with at least about: 70%, 75%, 80%, 85%, 87%, 90%, 91%, 92%, 93%, 94%, 95%, 96%, 97%, 98%, 99%, 99.5%, or 100% sequence identity to the 16SrRNA and/or 23S rRNA of a microorganism selected from the group consisting of: Clostridium indolis, Bifidobacterium longum, and Akkermansia muciniphila.

In some embodiments, provided are therapeutic compositions comprising an isolated and/or purified microorganism population consisting of bacteria with at least about: 70%, 75%, 80%, 85%, 87%, 90%, 91%, 92%, 93%, 94%, 95%, 96%, 97%, 98%, 99%, 99.5%, or 100% sequence identity to the 16SrRNA and/or 23S rRNA of a microorganism selected from the group consisting of: Akkermansia muciniphila, Bifidobacterium infantis, Clostridium beijerinckii, Clostridium butyricum, and Eubacterium hallii, and any combination thereof.

In some embodiments, provided are therapeutic compositions comprising an isolated and/or purified microorganism population consisting of bacteria with at least about: 70%, 75%, 80%, 85%, 87%, 90%, 91%, 92%, 93%, 94%, 95%, 96%, 97%, 98%, 99%, 99.5%, or 100% sequence identity to the 16SrRNA and/or 23S rRNA of a microorganism selected from the group consisting of: Akkermansia muciniphila, Clostridium beijerinckii, Clostridium butyricum, Eubacterium hallii, Bifidobacterium infantis, and any combination thereof.

The population may comprise an isolated and purified microbe with a ribosomal RNA (rRNA) sequence comprising at least about 70%, 75%, 80%, 85%, 87%, 90%, 91%, 92%, 93%, 94%, 95%, 96%, 97%, 98%, 99%, 99.5%, or 100% sequence identity to a rRNA sequence from Akkermansia muciniphila.

The population may comprise an isolated and purified microbe with a ribosomal RNA (rRNA) sequence comprising at least about 70%, 75%, 80%, 85%, 87%, 90%, 91%, 92%, 93%, 94%, 95%, 96%, 97%, 98%, 99%, 99.5%, or 100% sequence identity to a rRNA sequence from Bifidobacterium adolescentis.

The population may comprise an isolated and purified microbe with a ribosomal RNA (rRNA) sequence comprising at least about 70%, 75%, 80%, 85%, 87%, 90%, 91%, 92%, 93%, 94%, 95%, 96%, 97%, 98%, 99%, 99.5%, or 100% sequence identity to a rRNA sequence from Bifidobacterium infantis.

The population may comprise an isolated and purified microbe with a ribosomal RNA (rRNA) sequence comprising at least about 70%, 75%, 80%, 85%, 87%, 90%, 91%, 92%, 93%, 94%, 95%, 96%, 97%, 98%, 99%, 99.5%, or 100% sequence identity to a rRNA sequence from Bifidobacterium longum.

The population may comprise an isolated and purified microbe with a ribosomal RNA (rRNA) sequence comprising at least about 70%, 75%, 80%, 85%, 87%, 90%, 91%, 92%, 93%, 94%, 95%, 96%, 97%, 98%, 99%, 99.5%, or 100% sequence identity to a rRNA sequence from Clostridium beijerinckii.

The population may comprise an isolated and purified microbe with a ribosomal RNA (rRNA) sequence comprising at least about 70%, 75%, 80%, 85%, 87%, 90%, 91%, 92%, 93%, 94%, 95%, 96%, 97%, 98%, 99%, 99.5%, or 100% sequence identity to a rRNA sequence from Clostridium butyricum.

The population may comprise an isolated and purified microbe with a ribosomal RNA (rRNA) sequence comprising at least about 70%, 75%, 80%, 85%, 87%, 90%, 91%, 92%, 93%, 94%, 95%, 96%, 97%, 98%, 99%, 99.5%, or 100% sequence identity to a rRNA sequence from Clostridium indolis.

The population may comprise an isolated and purified microbe with a ribosomal RNA (rRNA) sequence comprising at least about 70%, 75%, 80%, 85%, 87%, 90%, 91%, 92%, 93%, 94%, 95%, 96%, 97%, 98%, 99%, 99.5%, or 100% sequence identity to a rRNA sequence from Eubacterium hallii.

### Methods for Determining Members of a Microbial Habitat

The present disclosure provides methods and compositions comprising microbial populations for the treatment of microbiome-related health conditions and/or disorders in a subject. Methods of the disclosure can include collection, stabilization and extraction of microbes for microbiome analysis. Methods of the disclosure can include determining the composition of a microbial habitat of a host to generate a microbiome profile. The composition of a microbial habitat can be used to diagnose a health condition of a host, for example, to determine likelihood of a disorder and/or treatment course of the disorder.

In some embodiments, methods of the disclosure can be used to determine microbial habitat of the gut or gastrointestinal tract of a subject. The gut comprises a complex microbiome including multiple species of microbes that can contribute to vitamin production and absorption, metabolism of proteins and bile acids, fermentation of dietary carbohydrates, and prevention of pathogen overgrowth. The composition of microbes within the gut can be linked to functional metabolic pathways in a subject. Non-limiting examples of metabolic pathways linked to gut microbiota include, energy balance regulation, secretion of leptin, lipid synthesis, hepatic insulin sensitivity, modulation of intestinal environment, and appetite signaling. Modification of the gut microbiome can increase the risk for health conditions such as ulcerative colitis, colorectal cancer, autoimmune disorders, obesity, diabetes, and inflammatory bowel disease.

In some embodiments, detection methods (e.g. sequencing) can be used to identify gut microbiome biomarkers associated with, for example, obesity and obesity-induced diabetes. For example, non-obese and obese subjects can be categorized based on differences in species of microbes present in their microbiome. Obese subjects can have reduced microbial diversity and higher levels of fermentation causing microbes, for example, bacteroidetes phylum and methanogenic archaea, compared with non-obese subjects. Subjects with obesity-induced diabetes can have a microbiota that promotes mass gain, metabolic endotoxemia, adipose tissue inflammation, and insulin resistance. Differences in microbes between obese and lean subjects can be used to generate microbial biomarker profiles associated with obesity that can be used to predict risk factors and/or treatment course.

In some embodiments, detection methods of the disclosure (e.g., sequencing) can be used to analyze changes in gut microbiome composition over time, for example, during antibiotic treatment, gut microbiome therapies, and various diets. The microbiome can be significantly altered upon exposure to antibiotics and diets that deplete the native microbial population. Methods of the disclosure can be used to generate profiles of the subject before and after administration of a therapeutic to characterize differences in the microbiota.

In some embodiments, methods to visualize the microbiome based on sequencing signatures are provided. In some embodiments, methods are provided to visualize the microbiome over time based on sequencing information.

Methods of the disclosure can be used to detect, characterize and quantify microbial habitat of the amniotic fluid of a pregnant woman. The amniotic cavity of a pregnant woman undergoing preterm labor can harbor genetic material from a greater diversity of microbes, including previously-uncharacterized microbes, compared with pregnant woman delivering at full-term. The microbial habit can be used to define the diversity and abundance of microbes invading the amniotic cavity in order to evaluate clinical significance and causal framework for preterm labor. The microbiome profiles of amniotic fluid of women with full-term delivery and preterm delivery can be compared to determine microbes that can be used as biomarkers for predicting and/or treating preterm labor.

Microorganisms can translocate from a mother to an infant through maternal mononuclear cells in breast milk, which may prime the developing infant immune system to appropriately respond to commensal and pathogenic bacteria. Methods of the disclosure can be used to determine microbial habitat of the gut of an infant to generate patterns of microbial colonization and effects of the microbes on development of immunity during infancy and early childhood.

Methods of the disclosure can be used to analyze microbial habitat of the skin. Parts of the skin, including cutaneous invaginations and appendages, sweat glands (eccrine and apocrine), sebaceous glands and hair follicles, can each be associated with unique microbiota. Comparison of skin microbiome profiles of a healthy subject and a subject with for example, acne, can provide insights into microbial involvement in skin health and disease.

A formulation can be customized for a subject. The composition can be formulated to comprise populations of isolated and purified microbiomes selected based on the host's microbiome. A custom formulation can comprise, for example, a prebiotic, a probiotic, an antibiotic, or a combination of active agents described herein. Data specific to the subject comprising for example age, gender, and weight can be combined with an analysis result to provide a therapeutic agent customized to the subject. For example, a subject's microbiome found to be low in a specific microbe relative to a sub-population of healthy subjects matched for age and gender can be provided with a therapeutic and/or cosmetic formulation comprising the specific microbe to match that of the sub-population of healthy subjects having the same age and gender as the subject.

### Animal Health

The nutritional requirements of animals may be achieved by supplementation of limiting nutrients. There may be at least about 20, 30, 40, 50, 60, 70, 80, 90, 100, 150, 200, 250, or 300 bacterial strains that occupy the gastrointestinal tracts of animals and humans. These bacteria may produce dangerous and toxic waste products that can result in bloating and gas, constipation, ulcers, diarrhea, and beneficial bacteria. The beneficial bacteria may create a positive effect on the well-being and health of animals through their autochthonous microflora. Probiotics may help promote the existence of specific strains in the gut and discourage less desirable ones. Since gut bacteria may have specific requirements for the nutrients, providing these nutrients may generate growth of gut bacteria.

### Computer Systems

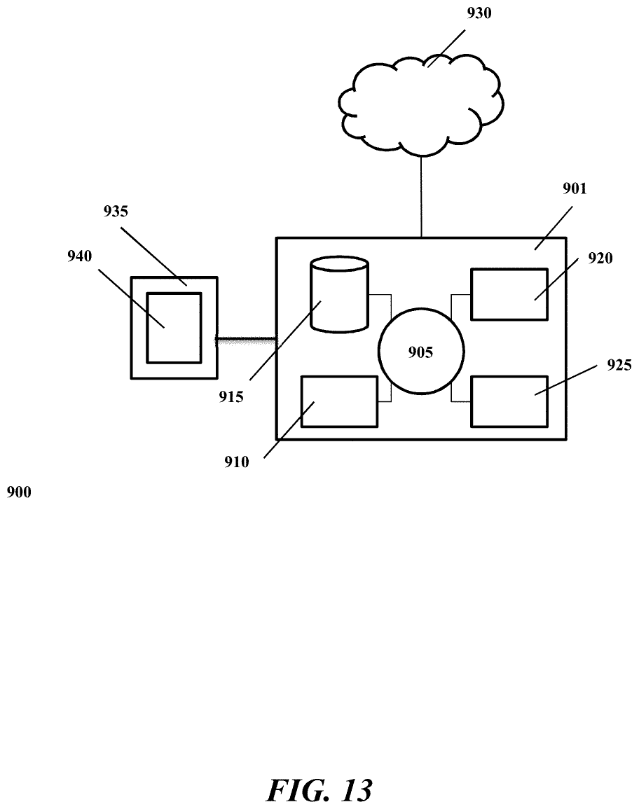

The disclosure also provides a computer system that is configured to implement the methods of the disclosure. The system can include a computer server (“server”) that is programmed to implement the methods described herein. FIG. 13 depicts a system 900 adapted to enable a user to detect, analyze, and process data (e.g. sequencing data; strain classification, functional pathways, epigenetic changes, patient information, external data, databases, microbiome strains; therapeutic consortia, etc.). The system 900 includes a central computer server 901 that is programmed to implement exemplary methods described herein. The server 901 includes a central processing unit (CPU, also “processor”) 905 which can be a single core processor, a multi core processor, or plurality of processors for parallel processing, or cloud processors. The server 901 also includes memory 910 (e.g. random access memory, read-only memory, flash memory); electronic storage unit 915 (e.g. hard disk); communications interface 920 (e.g. network adaptor) for communicating with one or more other systems; and peripheral devices 925 which may include cache, other memory, data storage, and/or electronic display adaptors. The memory 910, storage unit 915, interface 920, and peripheral devices 925 are in communication with the processor 1005 through a communications bus (solid lines), such as a motherboard. The storage unit 1015 can be a data storage unit for storing data. The server 901 is operatively coupled to a computer network (“network”) 930 with the aid of the communications interface 920. The network 930 can be the Internet, an intranet and/or an extranet, an intranet and/or extranet that is in communication with the Internet, a telecommunication or data network. The network 930 in some cases, with the aid of the server 901, can implement a peer-to-peer network, which may enable devices coupled to the server 901 to behave as a client or a server. Peripheral devices can include, e.g. sequencers 925 or remote computer systems 940.

The storage unit 915 can store files, (e.g. any aspect of data associated with the disclosure). In some instances cloud storage is used. Cloud storage can be a model of data storage where the digital data is stored in logical pools, wherein the physical storage can span multiple servers and, in some instances, one or more locations. In some embodiments, the physical environment is owned and managed by a hosting company. Cloud storage services may be accessed, e.g., through a co-located cloud compute service, a web service application programming interface (API) or by applications that utilize the API, such as cloud desktop storage, a cloud storage gateway or Web-based content management systems.

The server can communicate with one or more remote computer systems through the network 930. The one or more remote computer systems may be, for example, personal computers, laptops, tablets, telephones, Smart phones, or personal digital assistants.

In some situations the system 900 includes a single server 901. In other situations, the system includes multiple servers in communication with one another through an intranet, extranet and/or the Internet.

The server 901 can be adapted to store information. Such information can be stored on the storage unit 915 or the server 901 and such data can be transmitted through a network.

Methods as described herein can be implemented by way of machine (e.g., computer processor) computer readable medium (or software) stored on an electronic storage location of the server 901, such as, for example, on the memory 910, or electronic storage unit 915. During use, the code can be executed by the processor 905. In some cases, the code can be retrieved from the storage unit 915 and stored on the memory 910 for ready access by the processor 905. In some situations, the electronic storage unit 915 can be precluded, and machine-executable instructions are stored on memory 910. Alternatively, the code can be executed on a second computer system 940.

Aspects of the systems and methods provided herein, such as the server 901, can be embodied in programming. Various aspects of the technology may be thought of as “products” or “articles of manufacture” typically in the form of machine (or processor) executable code and/or associated data that is carried on or embodied in a type of machine readable medium (e.g., computer readable medium). Machine-executable code can be stored on an electronic storage unit, such memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as used across physical interfaces between local devices, through wired and optical landline networks and over various air-links. The physical elements that carry such waves, such as wired or wireless likes, optical links, or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, tangible storage medium, a carrier wave medium, or physical transmission medium. Non-volatile storage media can include, for example, optical or magnetic disks, such as any of the storage devices in any computer(s) or the like, such may be used to implement the system. Tangible transmission media can include: coaxial cables, copper wires, and fiber optics (including the wires that comprise a bus within a computer system). Carrier-wave transmission media may take the form of electric or electromagnetic signals, or acoustic or light waves such as those generated during radio frequency (RF) and infrared (IR) data communications. Common forms of computer-readable media therefore include, for example: a floppy disk, a flexible disk, hard disk, magnetic tape, any other magnetic medium, a CD-ROM, DVD, DVD-ROM, any other optical medium, punch cards, paper tame, any other physical storage medium with patterns of holes, a RAM, a ROM, a PROM and EPROM, a FLASH-EPROM, any other memory chip or cartridge, a carrier wave transporting data or instructions, cables, or links transporting such carrier wave, or any other medium from which a computer may read programming code and/or data. Many of these forms of computer readable media may be involved in carrying one or more sequences of one or more instructions to a processor for execution.

### EXAMPLES

### Example 1: Growth of High Concentrated Strains

FIG. 2 shows the optimal density measurements over time for the successful GMP growth of Akkermansia muciniphila in vegetable infusion. Vegetable infusion can increase growth of a microbe over time by at least about: 5-fold, 10-fold, 15-fold, 20-fold, 25-fold, 30-fold, 40-fold, 50-fold, 60-fold, 70-fold, 80-fold, 90-fold, or 100-fold compared with growth of the microbe in the absence of vegetable infusion as determined, for example, by optical density measurements. In some cases, vegetable infusion can increase growth of a microbe by at least 10-fold compared with growth of the microbe in the absence of vegetable infusion. Vegetable infusion can comprise one or more sugars such as an amino sugars and/or hexose sugars (e.g., dextrose); salts; proteins and/or amino acid sources (e.g., vegetable proteins; peptones); buffer; vitamins; reducing agents (e.g., L-cysteine); and antifoam agents.

### Example 2: Lyophilization into Stable Powder

In FIG. 9A, viable bacterial cell counts of B. longum in a 96 well plate are compared before and after lyophilization. Furthermore in FIG. 9A, a standard may be developed in plotting cycle threshold (Ct) value against the microbial dilutions. The standard may be used to determine cell counts based on the optical density of a microbial culture.

### Example 3: Strain Stability at Room Temperature and 4 Degrees Celsius

In FIG. 4, shows the stability of lyophilized compositions of isolated and purified strains as determined by counting total active cells in compositions of lyophilized isolated and purified microbes stored under different temperature conditions for 30 days. Strain 1, strain 2, and strain 3 are each obligate anaerobes. Strain 1, strain 2, and strain 3 were stored at 4 degrees Celsius and room temperature. Strain 1 remained stable for 30 days when stored at either room temperature or 4 degrees Celsius, as indicated by total active cell counts. Strain 3 remained stable in when stored at either 4 degrees Celsius or at room temperature. Strain 2 was determined to have greater stability when stored at 4 degrees Celsius than at room temperature.

FIG. 5A shows the stability of an encapsulated formulation of lyophilized obligate anerobes as determined by measuring active cells/g over time. FIG. 5B shows the stability of formulations over time (in number of active cells) when stored at 4 degrees Celsius.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1-65. (canceled)

66. A composition for administration to a subject in need thereof, comprising:
a population of microbes comprising obligate anaerobic microbes, wherein the obligate anaerobic microbes are from a plurality of microbial species, wherein at least 0.1% of the obligate anaerobic microbes are viable at 4 degrees Celsius or at 25 degrees Celsius for at least 14 days, and
wherein the composition is substantially dry and has a residual moisture of 5% or less.

67. The composition of claim 66, wherein at least 1% of the obligate anaerobic microbes are viable microbes or active cells.

68. The composition of claim 66, wherein the population is encapsulated and wherein at least 20% of the obligate anaerobic microbes are viable microbes or active cells.

69. The composition of claim 66, wherein 5% to 75% of the obligate anaerobic microbes are viable microbes or active cells.

70. The composition of claim 66, wherein the composition comprises an amount of at least 10⁵ viable cells/g of at least one of the microbial species.

71. The composition of claim 66, wherein the composition comprises an amount of at least 10⁸ viable cells/g of at least one of the microbial species.

72. The composition of claim 66, wherein the composition is a powder.

73. The composition of claim 66, wherein the obligate anaerobic microbes are viable in greater than 5 μM oxygen.

74. The composition of claim 66, wherein the obligate anaerobic microbes are viable in conditions comprising 20% or greater oxygen, by volume.

75. The composition of claim 66, wherein at least one of the plurality of microbial species is capable of growing in conditions having 5 μM or less of dissolved oxygen.

76. The composition of claim 66, wherein 90% or more of the obligate anaerobic microbes are non-sporulated.

77. The composition of claim 66, wherein the population comprises an isolated and purified microbe with a ribosomal RNA (rRNA) sequence comprising at least about 95% sequence identity to a rRNA sequence from Akkermansia muciniphila.

78. The composition of claim 66, wherein the population comprises an isolated and purified microbe with a ribosomal RNA (rRNA) sequence comprising at least about 95% sequence identity to a rRNA sequence from Bifidobacterium infantis.

79. The composition of claim 66, wherein the population comprises an isolated and purified microbe with a ribosomal RNA (rRNA) sequence comprising at least about 95% sequence identity to a rRNA sequence from Clostridium beijerinckii.

80. The composition of claim 66, wherein the population comprises an isolated and purified microbe with a ribosomal RNA (rRNA) sequence comprising at least about 95% sequence identity to a rRNA sequence from Clostridium butyricum.

81. The composition of claim 66, wherein the population comprises an isolated and purified microbe with a ribosomal RNA (rRNA) sequence comprising at least about 95% sequence identity to a rRNA sequence from Eubacterium hallii.

82. The composition of claim 66, wherein the population comprises at least one microbe from genus Akkermansia and at least one microbe from a genus selected from the group consisting of: Eubacterium, Clostridium, Bifidobacterium, and Faecalibacterium.

83. The composition of claim 66, wherein the population comprises Akkermansia muciniphila and Eubacterium hallii.

84. The composition of claim 66, wherein the population comprises Akkermansia muciniphila, Clostridium beijerinckii, and Eubacterium hallii.

85. The composition of claim 66, wherein the population comprises Clostridium beijerinckii, Clostridium butyricum, and Bifidobacterium infantis.

86. The composition of claim 66, wherein the population comprises Akkermansia muciniphila, Bifidobacterium infantis, Clostridium beijerinckii, Clostridium butyricum, and Eubacterium hallii.

87. The composition of claim 66, further comprising a prebiotic.

88. The composition of claim 87, wherein the prebiotic comprises inulin.

89. The composition of claim 66, wherein the composition is formulated for oral delivery.

90. The composition of claim 66, wherein the composition is a tablet or capsule.

91. The composition of claim 90, wherein the tablet or capsule further comprises one or more enteric coating(s).

92. The composition of claim 66, wherein the composition is a food or dietary supplement.
